(19) United States
(12) Patent Application Publication
(10) Pub. No.: US 2009/0318456 A1
(43) Pub. Date: Dec. 24, 2009

(54) SUBSTITUTED PTERIDINES FOR THE TREATMENT AND PREVENTION OF VIRAL INFECTIONS

(75) Inventors: Piet André Maurits Maria Herdewijn, Rotselaar/Wezemaal (BE); Steven Cesar Alfons De Jonghe, Brussel (BE); William A. Lee, Los Altos, CA (US); William John Watkins, Saratoga, CA (US); Steven S. Bondy, Danville, CA (US); Lee S. Chong, Newark, CA (US)

Correspondence Address: MARIA HERDEWIJN, OLIVIERSTRAAT 21, ROTSELAAR/WEZEMALL, B-3111, BE

(73) Assignee: Gilead Sciences, Inc., Foster City, CA (US)

(21) Appl. No.: 12/307,727

(22) PCT Filed: Jul. 6, 2007
(86) PCT No.: PCT/BE07/00076

§ 371 Date: Jan. 6, 2009

Related U.S. Application Data
(60) Provisional application No. 60/806,672, filed on Jul. 6, 2006.

(51) Int. Cl. A61K 31/4985; A61P 31/12; C07D 475/08

(52) U.S. Cl. 514/249; 544/260

(57) ABSTRACT

Tri-substituted pteridines and tetra-substituted pteridines exhibit a significant and selective activity against certain types of viral infections, in particular they selectively inhibit the replication of the hepatitis C virus, and are useful for the prevention and treatment of such infections.

### FIELD OF THE INVENTION

The present invention relates to the use of certain classes of specifically substituted pteridine derivatives as biologically active ingredients for manufacturing medicaments for the prevention or treatment of infections by a virus of the Flaviridae family, more specifically for inhibiting replication of hepatitis C virus. The present invention thus also relates to therapeutic and prophylactic methods comprising administration of said specifically substituted pteridine derivatives, or pro-drugs thereof, to mammals, in particular human beings.

### BACKGROUND OF THE INVENTION

There is a continuous need in the art for specific and highly therapeutically active compounds for preventing or treating infections due to Flaviridae and pathologic conditions associated therewith, especially hepatitis C. In particular, there is a need in the art to provide drugs which are active against hepatitis C in a minor dose in order to replace existing drugs having significant side effects and to decrease treatment costs.

Hepatitis is an inflammation of the liver that is most often caused by infection with one of three viruses known as hepatitis A, B or C. Hepatitis A virus (HAV) infection is the most common cause of acute hepatitis, and usually resolves spontaneously after several weeks of acute symptoms. Hepatitis B virus (HBV) and hepatitis C virus (HCV) are the most common viral causes of chronic hepatitis, usually defined as liver inflammation persisting for more than six months. HCV is the second most common cause of viral hepatitis in general and most common cause of chronic hepatitis. The World Health Organization estimates that worldwide 170 million people (3% of the world's population) are chronically infected with HCV. These chronic carriers are at risk of developing cirrhosis and/or liver cancer. In studies with a 10 to 20 year follow-up, cirrhosis developed in 20-30% of the patients, 1-5% of whom may develop liver cancer during the next then years. The 15% to 45% of persons with acute hepatitis C who do recover are not subject to long-term complications and do not need treatment. Since HCV and pestiviruses belong to the same virus family and share many similarities (such as, but not limited to, organization of the genome, analogous gene products and replication cycle), pestiviruses may be adopted as a model virus and surrogate for HCV. For example the Bovine Viral Diarrhea Virus (BVDV) is closely related to hepatitis C virus (HCV) and may be used as a surrogate virus in drug development for HCV infection.

HCV is a representative and highly significant member of the Flaviviridae family, a family of positive-strand RNA viruses. This family includes the following genera: Genus Flavivirus (type species Yellow fever virus, others include West Nile virus and Dengue Fever), Genus Hepacivirus (type species Hepatitis C virus), and Genus Pestivirus (type species Bovine viral diarrhea virus (BVDV), others include classical swine fever or hog cholera). Contrary to other families of positive strand RNA viruses such as human immunodeficiency virus (HIV), HCV seems incapable of integrating into the host's genome. The primary immune response to HCV is mounted by cytotoxic T lymphocytes. Unfortunately, this process fails to eradicate infection in most people; in fact, it may contribute to liver inflammation and, ultimately, tissue necrosis. The ability of HCV to escape immune surveillance is the subject of much speculation. One likely means of viral persistence relies on the presence of closely related but heterogeneous populations of viral genomes. Further studies of these quasi-species enable classification of several genotypes and subtypes, which have clinical implications.

The diagnosis of hepatitis C is rarely made during the acute phase of the disease because the majority of people infected experience no symptoms during this phase of the disease. Those who do experience acute phase symptoms are rarely ill enough to seek medical attention. The diagnosis of chronic phase hepatitis C is also challenging due to the absence or lack of specificity of symptoms until advanced liver disease develops, which may not occur until decades into the disease.

Hepatitis C testing begins with serological blood tests used to detect antibodies to HCV. Anti-HCV antibodies can be detected in about 80% of patients within 15 weeks after exposure, in more than 90% of patients within 5 months after exposure, and in more than 97% of patients by 6 months after exposure. Overall, HCV antibody tests have a strong positive predictive value for exposure to the hepatitis C virus, but may miss patients who have not yet developed antibodies (seroconversion), or have an insufficient level of antibodies to detect. Anti-HCV antibodies indicate exposure to the virus, but cannot determine if ongoing infection is present. All persons with positive anti-HCV antibody tests must undergo additional testing for the presence of the hepatitis C virus itself to determine whether current infection is present. The presence of HCV may be tested by using molecular nucleic acid testing methods such as, but not limited to, polymerase chain reaction (PCR), transcription mediated amplification (TMA), or branched DNA amplification. All HCV nucleic acid molecular tests have the capacity to detect not only whether the virus is present, but also to measure the amount of virus present in the blood (the HCV viral load). The HCV viral load is an important factor in determining the probability of response to interferon-base therapy, but does not indicate disease severity nor the likelihood of disease progression.

The goal of treatment is to prevent complications of HCV infection. This is principally achieved by eradication of infection. Accordingly, treatment responses are frequently characterized by the results of HCV RNA testing. Infection is considered eradicated when there is a sustained virologic response (SVR), defined as the absence of HCV RNA in serum by a sensitive test at the end of treatment and 6 months later. Persons who achieve an SVR almost always have a dramatic earlier reduction in the HCV RNA level, referred to as an early virologic response (EVR). Continued absence of detectable virus at termination of treatment is referred to as end of treatment response (ETR). A patient is considered relapsed when HCV RNA becomes undetectable on treatment but is detected again after discontinuation of treatment. Persons in whom HCV RNA levels remain stable on treatment are considered as non-responders, while those whose HCV RNA levels decline but remain detectable are referred to as partial responders.

Current standard of care for HCV treatment is a combination of (pegylated) interferon alpha and the antiviral drug ribavirin for a period of 24 or 48 weeks, depending upon the viral genotype. Should treatment with pegylated ribavirin-interferon not return a viral load reduction after 12 weeks, the chance of treatment success is less than 1%. Current indication for treatment includes patients with proven hepatitis C virus infection and persistent abnormal liver function tests. SVR of 75% or better occur in people with genotypes HCV 2 and 3 within 24 weeks of treatment, about 50% in those with genotype 1 within 48 weeks of treatment and 65% for those with genotype 4 within 48 weeks of treatment. About 80% of hepatitis C patients in the United States exhibit genotype 1, whereas genotype 4 is more common in the Middle East and Africa.

Best results have been achieved with the combination of weekly subcutaneous injections of long-acting peginterferon alpha and oral ribavirin daily. Interferons are substances naturally released by cells in the body after viral invasion. Interferon alfa-2b and peginterferon alfa-2b are synthetic versions of these substances. The protein product is manufactured by recombinant DNA-technology. Second generation interferons are further derivatized by binding to inert polyethylene glycol, thereby altering the pharmacokinetic properties. Ribavirin is a nucleoside analogue, which disrupts viral replication of hepatitis C virus (HCV).

The most common side effects of HCV treatment with (pegylated) interferon include: a decrease in white blood cells and platelets, anemia, nausea, diarrhea, fever, chills, muscle and joint pain, difficulty in concentrating, thyroid dysfunction, hair loss, sleeplessness, irritability, mild to serious depression, and rarely, suicidal thoughts. Other serious adverse events include bone marrow toxicity, cardiovascular disorders, hypersensitivity, endocrine disorders, pulmonary disorders, colitis, pancreatitis, and opthalmologic disorders (eye and vision problems). (Pegylated) interferon may also cause or make worse fatal or life-threatening neuropsychiatric, autoimmune, ischemic, and infectious disorders. Patients with persistently severe or worsening signs or symptoms of these conditions are advised to stop therapy.

The most common side effect of HCV treatment with ribavirin is anemia, which can be treated with erythropoietin. Other side effects include mood swings, irritability, anxiety, insomnia, abdominal pain, nervousness, breathlessness, rash, hair loss, dry skin, nausea, diarrhoea, loss of appetite, dizziness and weight loss. Ribavirin can also cause birth defects. Ribavirin should not be taken in combination with certain HIV drugs such as, but not limited to, didanosine, since lactic acidosis with fatal hepatic steatosis (fatty liver) may occur. Special attention should be taken for treatment with HIV co-infection.

Although the liver is the primary target of infection, studies to better define the steps of HCV infection are greatly hampered by the lack of a suitable animal model for such studies. The recent development of sub-genomic HCV RNA replicons capable of autonomous replication in the human hepatoma cell line, Huh-7, has been a significant advance in the study of HCV biology. The sub-genomic HCV RNA replicon system provides a cell-based assay to evaluate inhibitors of HCV enzymes like the protease, helicase, and RNA-dependant RNA polymerase or to evaluate nucleic acid targeting strategies like antisense RNA and ribozymes.

Targets for HCV Drug development include HCV-encoded enzymes, namely, NS2-3 and NS3-4A proteases, NS3 helicase, and NS5B RNA dependant RNA polymerase. Alternatively, HCV replication can be inhibited by blocking the conserved RNA elements employing a nucleic acid based approach including antisense oligonucleotides, ribozymes, RNA aptamers, RNA decoys, and RNA interference. A major drawback for such nucleic acid based approach is the size and charge of the nucleic acids, and their usually low physiological stability that do not allow for oral administration. Another target option for therapy is by blocking viral entry into the cell by obstruction of binding to HCV receptors such as, but not limited to, CD 209L and L-SIGN.

There is a strong need in the art to improve, or to provide alternatives to, the existing prophylactic or therapeutic solutions to infections by a virus of the Flaviridae family, more specifically HCV infection. In particular there is still a need in the art for providing alternative synthetic molecules having significant HCV replication inhibiting activity. There is also a need in the art for providing effective inhibiting molecules which are free from the significant drawbacks of the current drugs like pegylated interferon and ribavirin. Meeting these various needs in the art constitutes the main goal of the present invention.

### SUMMARY OF THE INVENTION

The present invention is based on the unexpected finding that a number of substituted pteridines, in particular trisubstituted pteridines and tetrasubstituted pteridines, which have been known in the art as immunosuppressive or immunomodulating agents, as well as having other useful biological properties, are also capable of exhibiting a significant and selective activity against certain types of viral infections, provided that the substituting pattern of such pteridines is suitably selected. In particular, these trisubstituted pteridines and tetrasubstituted pteridines are capable of selectively inhibiting the replication of the hepatitis C virus. It has been surprisingly found that their activity is virus-specific, especially since they do not exhibit activity against other families of positive strand RNA viruses such as human immunodeficiency virus (HIV-1 or HIV-1-2).

### GENERAL DESCRIPTION OF THE INVENTION

An aspect of the present invention is a method of treatment or prevention of an infection due to a virus from the Flaviridae family, in particular HCV, by administering to a patient in need thereof a therapeutically effective amount of a pteridine derivative having the structural formula (I):

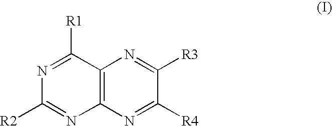

wherein:
R₁ is selected from the group consisting of hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl;
R₂ is selected from the group consisting of amino, hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl,
R₃ is selected from the group consisting of unsubstituted, monosubstituted and disubstituted aryl groups wherein the optional substituent(s) may be, but are not limited to, halogen, C₁₋₄ alkoxy, C₁₋₄ alkyl, aryl groups bonded to the pteridine ring via a saturated or unsaturated aliphatic spacer which may be halogenated or hydroxylated, and aliphatic substituents which may contain an ether, hydroxy, amino or C₁₋₄ alkoxy group; and
R₄ is selected from the group consisting of hydrogen, alkyl, alkoxy, substituted and non-substituted aryl groups,
   with the proviso that said pteridine derivative is not 2-amino-4-ethoxy-6-(4-fluorophenyl)-pteridine or 2-amino-4-isopropoxy-6-(4-fluorophenyl)-pteridine, and/or a pharmaceutically acceptable addition salt thereof, and/or a stereoisomer thereof, and/or a mono- or a di-N-oxide thereof, and/or a solvate thereof, and/or a dihydro- or tetrahydropteridine derivative thereof, and/or a pro-drug thereof.

Also an aspect of the present invention is the use of a pteridine derivative represented by formula (I) as defined herein, in the manufacture of a medicament for the treatment or prevention of an infection due to a virus of the Flaviviridae family, in particular due to Hepatitis C virus, and also in particular of a medicament for oral administration.

Another aspect of the present invention is a method of treatment or prevention of an infection due to a virus from the Flaviridae family, in particular HCV, by administering to a patient in need thereof a therapeutically effective amount of a pteridine derivative having the structural formula (II)

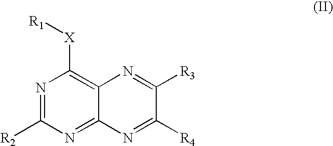

wherein X represents an oxygen atom or a group with the formula S(O)ₘ wherein m is an integer from 0 to 2, or a group with the formula NZ and wherein:
R₁ is a group selected from the group consisting of C₁₋₇ alkyl, C₂₋₇ alkenyl, C₂₋₇ alkynyl, C₃₋₁₀ cycloalkyl, C₃₋₁₀ cycloalkenyl, aryl, alkylaryl, arylalkyl, heterocyclic, heterocyclic-substituted alkyl and alkyl-substituted heterocyclic, each of said groups being optionally substituted with one or more substituents selected from the group consisting of halogen, C₁₋₄ alkyl, C₁₋₄ alkoxy, C₂₋₇ alkenyl, C₂₋₇ alkynyl, halo C₁₋₄ alkyl, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxyl, sulfhydryl, nitro, hydroxylamino, mercaptoamino, cyano, carboxylic acid or esters or thioesters or amides or thioamides or halides or anhydrides thereof, thiocarboxylic acid or esters or thioesters or amides or thioamides or halides or anhydrides thereof, carbamoyl, thiocarbamoyl, ureido, thio-ureido, amino, alkylamino, cycloalkylamino, alkenylamino, cycloalkenyl-amino, alkynylamino, arylamino, arylalkylamino, hydroxyalkylamino, mercaptoalkylamino, heterocyclic amino, hydrazino, alkylhydrazino and phenyl-hydrazino; or R₁ is a carboxyalkyl, carboxyaryl, thiocarboxyaryl or thiocarboxyalkyl group;
Z is a group independently defined as R₁ or Z is hydrogen or the group NZ together with R₁ is either hydroxylamino or an optionally substituted heterocyclic group containing at least one nitrogen atom;
R₂ is selected from the group consisting of amino; acylamino; thioacylamino; carbamoyl; thiocarbamoyl, ureido; thio-ureido, sulfonamido; hydroxylamino; alkoxyamino; thioalkylamino; mercaptoamino, hydrazino; alkylhydrazino, phenylhydrazino; optionally substituted heterocyclic radicals; C₃₋₇ alkylamino; arylamino; arylalkylamino; cycloalkylamino; alkenylamino; cycloalkenylamino; heterocyclic amino; hydroxyalkylamino; mercaptoalkylamino; C₁₋₇ alkoxy; C₃₋₁₀ cycloalkoxy; thio C₁₋₇ alkyl; arylsulfoxide; arylsulfone; heterocyclic sulfoxide; heterocyclic sulfone; thio C₃₋₁₀ cycloalkyl; aryloxy; arylthio; arylalkyloxy; arylalkylthio; oxyheterocyclic and thioheterocyclic radicals;
R₄ is an atom or a group selected from the group consisting of hydrogen; halogen; C₁₋₇ alkyl; C₂₋₇ alkenyl; C₂₋₇ alkynyl; halo C₁₋₇ alkyl; carboxy C₁₋₇ alkyl; carboxyaryl; C₁₋₇ alkoxy; C₃₋₁₀ cycloalkoxy; aryloxy; arylalkyloxy; oxyheterocyclic; heterocyclic-substituted alkyloxy; thio C₁₋₇ alkyl; thio C₃₋₁₀ cycloalkyl; thioaryl; thioheterocyclic; arylalkylthio; heterocyclic-substituted alkylthio; hydroxylamino; mercapto-amino; acylamino; thio-acylamino; alkoxyamino; thioalkylamino; acetal; thio-acetal; carboxylic acid; carboxylic acid esters, thioesters, halides, anhydrides, amides and thioamides; thiocarboxylic acid; thiocarboxylic acid esters, thioesters, halides, anhydrides, amides and thioamides; hydroxyl; sulfhydryl; nitro; cyano; carbamoyl; thiocarbamoyl, ureido; thio-ureido; alkylamino; cycloalkyl-amino; alkenylamino; cycloalkenylamino; alkynylamino; arylamino; arylalkylamino; hydroxyalkylamino; mercaptoalkylamino; heterocyclic amino; heterocyclic-substituted alkylamino; oximino; alkyloximino; hydrazino; alkylhydrazino; phenylhydrazino; cysteinyl acid, esters, thioesters, halides, anhydrides, amides and thioamides thereof; phenyl substituted with one or more substituents selected from the group consisting of C₁₋₇ alkyl, C₂₋₇ alkenyl, C₂₋₇ alkynyl, halo C₁₋₇ alkyl, nitro, hydroxyl, sulfhydryl, amino, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, carbamoyl, thiocarbamoyl, ureido, thio-ureido, sulfonamido, hydroxylamino, alkoxyamino, mercaptoamino, thioalkyl-amino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cycloalkenylamino, alkynyl-amino, arylamino, arylalkylamino, hydroxyalkylamino, mercaptoalkylamino, heterocyclic amino, hydrazino, alkylhydrazino and phenylhydrazino; aryl groups other than phenyl, the said aryl groups being optionally substituted with one or more substituents selected from the group consisting of halogen, C₁₋₇ alkyl, C₁₋₇ alkoxy, C₂₋₇ alkenyl, C₂₋₇ alkynyl, halo C₁₋₇ alkyl, nitro, hydroxyl, sulfhydryl, amino, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, carbamoyl, thiocarbamoyl, ureido, thio-ureido, sulfonamido, hydroxylamino, alkoxyamino, mercaptoamino, thioalkyl-amino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cycloalkenylamino, alkynylamino, arylamino, arylalkylamino, hydroxyalkylamino, mercaptoalkylamino, heterocyclic amino, hydrazino, alkylhydrazino and phenylhydrazino; optionally substituted heterocyclic radicals e.g. selected from the group consisting of oxabicycloheptyl, azabenzimidazolyl, azacycloheptyl, azacyclooctyl, azacyclononyl, azabicyclononyl, tetrahydrofuryl, tetrahydro-pyranyl, tetrahydropyronyl, tetrahydroquinoleinyl, tetrahydro-thienyl and dioxide thereof, dihydrothienyl dioxide, dioxindolyl, dioxinyl, dioxenyl, dioxazinyl, thioxanyl, thioxolyl, thio-urazolyl, thiotriazolyl, thiopyranyl, thiopyronyl, coumarinyl, quinoleinyl, oxyquinoleinyl, quinuclidinyl, xanthi-nyl, dihydropyranyl, benzodihydrofuryl, benzothiopyronyl, benzothio-pyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl, phenopyronyl, phenoxazolyl, pyridinyl, dihydropyridinyl, tetrahydropyridinyl, piperidinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofuryl, furoyl, hydantoinyl, dioxolanyl, dioxolyl, dithianyl, dithienyl, dithiinyl, thienyl, indolyl, indazolyl, benzofuryl, quinolyl, quinazolinyl, quinoxalinyl, carbazolyl, phenoxazinyl, phenothiazinyl, xanthenyl, purinyl, benzothienyl, naphtothienyl, thianthrenyl, pyranyl, pyronyl, benzopyronyl, isobenzo-furanyl, chromenyl, phenoxathiinyl, indolizinyl, quinolizinyl, isoquinolyl, phthalazinyl, naphthiridinyl, cinnolinyl, pteridinyl, carbolinyl, acridinyl, perimidinyl, phenanthrolinyl, phenazinyl, phenothiazinyl, imidazolinyl, imidazolidinyl, benzimidazolyl, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, thietyl, thietanyl, diazabicyclo-octyl, diazetyl, diaziridinonyl, diaziridinethionyl, chromanyl, chromanonyl, thiochromanyl, thiochromanonyl, thiochromenyl, benzofuranyl, benziso-thiazolyl, benzocarbazolyl, benzochromonyl, benzisoalloxazinyl, benzocoumarinyl, thiocoumarinyl, phenometoxazinyl, phenoparoxazinyl, phentriazinyl, thiodiazinyl, thiodiazolyl, indoxyl, thioindoxyl, benzodiazinyl, phtalidyl, phtalimidinyl, phtalazonyl, alloxazinyl, xanthionyl, isatyl, isopyrazolyl, isopyrazolonyl, urazolyl, urazinyl, uretinyl, uretidinyl, succinyl, succinimido, benzylsultimyl and benzylsultamyl; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals selected from the group consisting of carbonyl (oxo), thiocarbonyl, alcohol (hydroxyl), thiol, ether, thio-ether, acetal, thio-acetal, amino, imino, oximino, alkyloximino, amino-acid, cyano, acylamino, thioacylamino, carbamoyl, thiocarbamoyl, ureido, thio-ureido, carboxylic acid or ester or thioester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkylamino, cycloalkylamino, alkenylamino, cycloalkenylamino, alkynylamino, arylamino, arylalkylamino, hydroxyalkylamino, mercaptoalkylamino, heterocyclic amino, hydrazino, alkylhydrazino, phenylhydrazino, sulfonyl, sulfinyl, sulfonamido and halogen; branched or straight, saturated or unsaturated aliphatic chains of 1 to 7 carbon atoms optionally containing one or more functions selected from the group consisting of carbonyl (oxo), thiocarbonyl, alcohol (hydroxyl), thiol, ether, thio-ether, acetal, thio-acetal, amino, imino, oximino, alkyloximino, aminoacid, cyano, acylamino; thioacylamino; carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkylamino, cycloalkylamino, alkenyl-amino, cycloalkenylamino, alkynylamino, arylamino, arylalkylamino, hydroxyalkylamino, mercaptoalkylamino, heterocyclic amino, hydrazino, alkylhydrazino, phenylhydrazino, sulfonyl, sulfinyl, sulfonamido and halogen; and
R₃ is an atom or a group selected from the group consisting of fluoro, bromo, iodo, C₂₋₇ alkyl; C₂₋₇ alkenyl; C₂₋₇ alkynyl; halo C₁₋₇ alkyl; C₁₋₇ alkoxy; C₃₋₁₀ cycloalkoxy; aryloxy; arylalkyloxy; oxyheterocyclic; heterocyclic-substituted alkyloxy; thio C₂₋₇ alkyl; thio C₃₋₁₀ cycloalkyl; thioaryl; thioheterocyclic; arylalkylthio; heterocyclic-substituted alkylthio; hydroxylamino; alkoxyamino; thioalkylamino; mercaptoamino; acylamino; thio-acylamino; thio-acetal; carboxylic acid; carboxylic acid esters, thioesters, amides, halides, anhydrides and thioamides; thiocarboxylic acid; thiocarboxylic acid esters, thioesters, amides, halides, anhydrides and thioamides; hydroxyl; sulfhydryl; nitro; carbamoyl; thiocarbamoyl; ureido; thio-ureido; amino; alkylamino; cycloalkylamino; alkenylamino; cycloalkenylamino; alkynylamino; arylamino; arylalkylamino; hydroxyalkyl-amino; mercaptoalkylamino; heterocyclic amino; heterocyclic-substituted alkylamino; oximino; alkyloximino; hydrazino; alkylhydrazino; phenyl-hydrazino; cysteinyl acid, esters, thioesters, amides and thioamides thereof; aryl optionally substituted with one or more substituents selected from the group consisting of halogen, C₁₋₇ alkyl, C₁₋₇ alkoxy, C₂₋₇ alkenyl, C₂₋₇ alkynyl, halo C₁₋₇ alkyl, nitro, hydroxyl, sulfhydryl, amino, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, carbamoyl, thiocarbamoyl, ureido, thio-ureido, sulfonamido, hydroxylamino, mercapto-amino, alkoxyamino, thioalkylamino, acylamino, thio-acylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cycloalkenylamino, alkynylamino, arylamino, arylalkylamino, hydroxy-alkylamino, mercaptoalkylamino, heterocyclic amino, hydrazino, alkylhydrazino and phenylhydrazino; optionally substituted heterocyclic radicals; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals selected from the group consisting of carbonyl (oxo), thiocarbonyl, alcohol (hydroxyl), thiol, ether, thio-ether, acetal, thio-acetal, amino, imino, oximino, alkyloximino, amino-acid, cyano, carboxylic acid or ester or thioester or amide, nitro, thio C₁₋₇ alkyl, thio C₃₋₁₀ cycloalkyl, alkylamino, cycloalkylamino, alkenylamino, cycloalkenylamino, alkynylamino, arylamino, arylalkylamino, hydroxy-alkylamino, mercaptoalkylamino, heterocyclic amino, hydrazino, alkyl-hydrazino, phenylhydrazino, sulfonyl, sulfonamido and halogen; branched or straight, saturated or unsaturated aliphatic chains of 2 to 7 carbon atoms optionally containing one or more functions selected from the group consisting of thiocarbonyl, alcohol (hydroxyl), thiol, ether, thio-ether, thio-acetal, amino, imino, oximino, alkyloximino, amino-acid, cyano, acylamino, thioacylamino, carbamoyl, thiocarbamoyl, ureido, thio-ureido, carboxylic acid or ester or thioester or halide or anhydride or amide, thio carboxylic acid or ester or thioester or halide or anhydride or amide, nitro, thio C₁₋₇ alkyl, thio C₃₋₁₀ cycloalkyl, hydroxylamino, mercaptoamino, alkylamino, cycloalkylamino, alkenylamino, cycloalkenylamino, alkynylamino, aryl-amino, arylalkylamino, hydroxy-alkylamino, mercaptoalkylamino, heterocyclic amino, hydrazino, alkylhydrazino, phenylhydrazino, sulfonyl, sulfinyl, sulfonamido and halogen; or R₃ together with R₄ forms a homocyclic or heterocyclic radical;
   with the proviso that said pteridine derivative is not 2-amino-4-ethoxy-6-(4-fluorophenyl)-pteridine or 2-amino-4-isopropoxy-6-(4-fluorophenyl)-pteridine, and/or a pharmaceutically acceptable addition salt thereof, and/or a stereoisomer thereof, and/or a mono- or a di-N-oxide thereof, and/or a solvate thereof, and/or a dihydro- or tetrahydropteridine derivative thereof, and/or a pro-drug thereof.

Also an aspect of the present invention is the use of a pteridine derivative represented by formula (II) as defined herein, in the manufacture of a medicament for the treatment or prevention of an infection due to a virus of the Flaviviridae family, in particular due to Hepatitis C virus, and also in particular of a medicament for oral administration.

Also an aspect of the present invention is a method of treatment or prevention of an infection due to a virus from the Flaviridae family, in particular HCV, by administering to a patient in need thereof a therapeutically effective amount of a pteridine derivative having the structural formula (III):

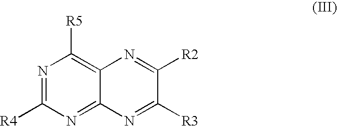

wherein:
a first group of one or more of the substituents R₂, R₃, R₄ and R₅ of the pteridine ring is independently selected from groups represented by the general formula (IV):

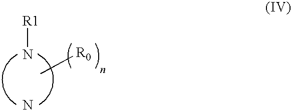

wherein:

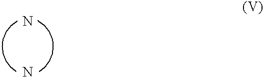

schematically represents a saturated or partly unsaturated heterocyclic ring with at least two nitrogen atoms in the said heterocyclic ring and with a total of 5 to 7 atoms in the said heterocyclic ring, and optionally with one or more other heteroatoms (e.g. oxygen or sulfur) in the said heterocyclic ring or attached to one or more carbon atoms of said heterocyclic ring (for instance in the form of a carbonyl or thiocarbonyl group), wherein one of said at least two nitrogen atoms in the heterocyclic ring is attached to a carbon atom of the pteridine ring at any of positions 2, 4, 6 or 7 of the pteridine ring, wherein the said heterocyclic ring may be fused to one or more aromatic hydrocarbon rings, and wherein:
each substituent R₀ of the heterocyclic ring (III) is a group independently selected from the group consisting of halogen, nitro, C₁₋₇ alkyl (optionally containing one or more functions or radicals selected from the group consisting of halogen, carbonyl, thiocarbonyl, hydroxyl, sulfhydryl, C₁₋₇ alkoxy, thio C₁₋₇ alkyl, thio C₃₋₁₀ cycloalkyl, acetal, thioacetal, imino, oximino, alkyloximino, amino-acid, cyano, (thio)carboxylic acid, (thio)carboxylic acid ester or amide, nitro, amino, C₁₋₇ alkylamino, cycloalkylamino, alkenylamino, cycloalkenyl-amino, alkynylamino, arylamino, arylalkylamino, hydroxyalkylamino, mercapto-alkylamino, heterocyclic-substituted alkylamino, heterocyclic amino, heterocyclic-substituted arylamino, hydrazino, alkylhydrazino, phenylhydrazino, sulfonyl and sulfonamido), C₃₋₇ alkenyl, C₂₋₇ alkynyl, halo C₁₋₇ alkyl, C₃₋₁₀ cycloalkyl, aryl, arylalkyl, alkylaryl, alkylacyl, arylacyl, hydroxyl, sulfhydryl, amino, C₁₋₇ alkylamino, cycloalkylamino, alkenylamino, cycloalkenylamino, alkynylamino, arylamino, arylalkyl-amino, hydroxyalkylamino, mercaptoalkylamino, heterocyclic-substituted alkylamino, heterocyclic amino, heterocyclic-substituted arylamino, hydrazino, alkylhydrazino, phenylhydrazino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxylamino, cyano, (thio)carboxylic acid or esters or thioesters or amides or thioamides thereof;
n is an integer from 0 to 6;
R₁ is a substituent group selected from the group consisting of formyl, acyl, thio-acyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cyclo-alkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfon-amido, hydroxylamino, alkoxyamino, mercaptoamino, thioalkyl-amino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenyl-amino, cycloalkenylamino, alkynylamino, arylamino, aryl-alkylamino, hydroxyalkylamino, mercaptoalkylamino, heterocyclic amino, hydrazino, alkylhydrazino and phenylhydrazino; and
wherein the remaining of, i.e. a second group of, the substituents R₂, R₃, R₄ and R₅ of the pteridine ring is independently selected from the group consisting of hydrogen; halogen; C₁₋₇ alkyl; C₂₋₇ alkenyl; C₂₋₇ alkynyl; halo C₁₋₇ alkyl; carboxy C₁₋₇ alkyl; carboxyaryl; C₁₋₇ alkoxy; C₃₋₁₀ cycloalkoxy; aryloxy; arylalkyloxy; oxyheterocyclic; heterocyclic-substituted alkyloxy; thio C₁₋₇ alkyl; thio C₃₋₁₀ cycloalkyl; thioaryl; thioheterocyclic; arylalkylthio; heterocyclic-substituted alkylthio; hydroxylamino; mercapto-amino; acylamino; thio-acylamino; alkoxyamino; thioalkyl-amino; acetal; thio-acetal; carboxylic acid; carboxylic acid esters, thioesters, halides, anhydri-des, amides and thioamides; thiocarboxylic acid; thiocarboxylic acid esters, thioesters, halides, anhydrides, amides and thioamides; hydroxyl; sulfhydryl; nitro; cyano; carbamoyl; thiocarbamoyl; ureido; thioureido; amino; alkylamino; cycloalkylamino; alkenylamino; cyclo-alkenylamino; alkynylamino; arylamino; arylalkylamino; hydroxyalkylamino; mercaptoalkyl-amino; heterocyclic amino; heterocyclic-substituted arylamino; heterocyclic-substituted alkyl-amino; oximino; alkyloximino; hydrazino; alkylhydrazino; phenylhydrazino; cysteinyl acid, esters, thioesters, halides, anhydrides, amides and thioamides thereof; aryl optionally substi-tuted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, carbamoyl, thiocarbamoyl, ureido, thio-ureido, sulfonamido, hydroxylamino, alkoxyamino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cycloalkenylamino, alkynylamino, arylamino, arylalkylamino, hydroxyalkyl-amino, mercaptoalkylamino, heterocyclic-substituted alkylamino, heterocyclic amino, hetero-cyclic-substituted arylamino, hydrazino, alkylhydrazino and phenylhydrazino; optionally substituted heterocyclic radicals; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals independently selected from the group consisting of carbonyl, thiocarbonyl, hydroxyl, thiol, ether, thioether, acetal, thioacetal, amino, imino, oximino, alkyloximino, amino-acid, cyano, acylamino, thioacylamino, carbamoyl, thiocarbamoyl, ureido, thioureido, carboxylic acid or ester or thioester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkylamino, cycloalkylamino, alkenylamino, cycloalkenyl-amino, alkynylamino, arylamino, arylalkylamino, hydroxyalkylamino, mercaptoalkylamino, heterocyclic-substituted alkylamino, heterocyclic amino, heterocyclic-substituted arylamino, hydrazino, alkylhydrazino, phenylhydrazino, sulfonyl, sulfinyl, sulfonamido and halogen; branched or straight, saturated or unsaturated aliphatic chains of 1 to 7 carbon atoms optionally containing one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino; thioacylamino; carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkyl-amino, hydroxyalkylamino, mercaptoalkylamino, heterocyclic-substituted alkylamino, hetero-cyclic amino, heterocyclic-substituted arylamino, hydrazino, alkylhydrazino, phenylhydrazino, sulfonyl, sulfinyl and sulfonamido; or R₂ together with R₃ and the carbon atoms in positions 6 and 7 of the pteridine ring forms a homocyclic or heterocyclic radical;
   and/or a pharmaceutically acceptable addition salt thereof, and/or a stereoisomer thereof, and/or a mono- or a di-N-oxide thereof, and/or a solvate thereof, and/or a dihydro- or tetrahydropteridine derivative thereof, and/or a pro-drug thereof.

Also an aspect of the present invention is the use of a pteridine derivative represented by formula (III) as defined herein, in the manufacture of a medicament for the treatment or prevention of an infection due to a virus of the Flaviviridae family, in particular due to Hepatitis C virus, and also in particular of a medicament for oral administration.

A further aspect of the present invention relates to novel pteridine derivative having

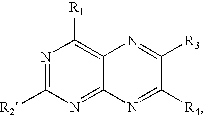

the structural formula (VI)

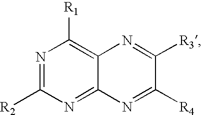

or the structural formula (VII)

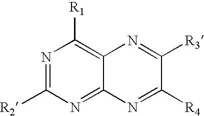

or the structural formula (VIII)
wherein:
R₁, R₂, R₃ and R₄ are as defined for formula (I) here above, and
R₂′ is —NH—CHR₆R₇ or —NH—R₈, wherein R₆ and R₇ are independently selected from the group consisting of hydrogen, C₁₋₆ alkyl substituted with one or more substituents selected from the group consisting of halogen and C₁₋₄ alkoxy, C₃₋₁₀ cycloalkyl, aryl and heterocyclyl, wherein said aryl is optionally substituted with one or more substituents selected from the group consisting of halogen, hydroxy, amino, nitro, cyano, trifluoromethyl, trifluoromethoxy, C₁₋₄ alkyl, C₁₋₄ alkoxy, di-C₁₋₄ alkylamino, mono-C₁₋₄ alkylamino, carboxamido, sulfamoyl, carbamoyl, sulfonamido and phenoxy provided that R₆ and R₇ are not both hydrogen, and wherein R₈ is selected from the group consisting of C₃₋₁₀ cycloalkyl substituted at the carbon position adjacent to the N atom of R₂′ with aryl or heteroaryl wherein said aryl is optionally substituted with halogen, heteroaryl, and heteroaryl or aryl wherein said heteroaryl or aryl is substituted with one or more substituents selected from the group consisting of halogen and C₁₋₄ alkyl;
R₃′ is selected from the group consisting of heterocyclyl, and, mono-substituted or disubstituted aryl and heterocyclyl, wherein at least one substituent of said aryl is selected from the group consisting of amino, C₄₋₆ alkyl, C₄₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy, and optionally further substituted with halogen, C₁₋₄ alkyl, C₁₋₄ alkoxy; and at least one substituent of said heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁ are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
   or a pharmaceutical acceptable addition salt or a stereochemical isomeric form thereof or a N-oxide thereof or a solvate thereof or a prodrug thereof.

Also an aspect of the present invention relates to novel pteridine derivatives having

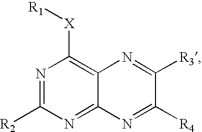

the structural formula (IX)

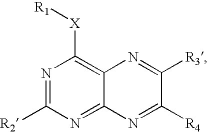

or the structural formula (X)

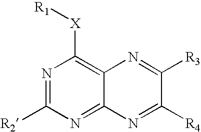

or the structural formula (XI)
wherein
X, R₁, R₂, R₃ and R₄ are as defined for formula (II) here above, and
R₂′ is —NH—CHR₆R₇ or —NH—R₈, wherein R₆ and R₇ are independently selected from the group consisting of hydrogen, C₁₋₆ alkyl substituted with one or more substituents selected from the group consisting of halogen and C₁₋₄ alkoxy, C₃₋₁₀ cycloalkyl, heterocyclyl, and aryl substituted with one or more substituents selected from the group consisting of halogen, hydroxy, amino, nitro, cyano, trifluoromethyl, trifluoromethoxy, C₁₋₄ alkyl, C₁₋₄ alkoxy, di-C₁₋₄ alkylamino, mono-C₁₋₄ alkylamino, carboxamido, sulfamoyl, carbamoyl, sulfonamido and phenoxy, provided that R₆ and R₇ are not both hydrogen, and wherein R₈ is selected from the group consisting of C₃₋₁₀ cycloalkyl optionally substituted at the carbon position adjacent to the N atom of R₂′ with aryl or heteroaryl wherein said aryl is optionally substituted with halogen, heteroaryl, and heteroaryl or aryl wherein said heteroaryl or aryl is substituted with one or more substituents selected from the group consisting of halogen and C₁₋₄ alkyl;
R₃′ is selected from the group consisting of heterocyclyl, and, mono-substituted or disubstituted aryl and heterocyclyl, wherein at least one substituent of said aryl is selected from the group consisting of amino, C₄₋₆ alkyl, C₄₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy, and optionally further substituted with halogen, C₁₋₄ alkyl, C₁₋₄ alkoxy; and at least one substituent of said heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁ are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
   or a pharmaceutical acceptable addition salt or a stereochemical isomeric form thereof or a N-oxide thereof or a solvate thereof or a prodrug thereof.

A further aspect of the present invention relates to pteridine derivatives having

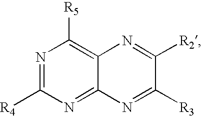

the structural formula (XII)

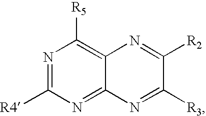

or the structural formula (XIII)

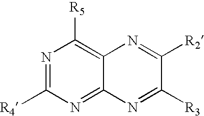

or the structural formula (XIV)
wherein
X, R₁, R₂, R₃ and R₄ are as defined for formula (III) here above, and
R₄′ is —NH—CHR₆R₇ or —NH—R₈, wherein R₆ and R₇ are independently selected from the group consisting of hydrogen, C₁₋₆ alkyl substituted with one or more substituents selected from the group consisting of halogen and C₁₋₄ alkoxy, C₃₋₁₀ cycloalkyl, heterocyclyl, and aryl substituted with one or more substituents selected from the group consisting of halogen, hydroxy, amino, nitro, cyano, trifluoromethyl, trifluoromethoxy, C₁₋₄ alkyl, C₁₋₄ alkoxy, di-C₁₋₄ alkylamino, mono-C₁₋₄ alkylamino, carboxamido, sulfamoyl, carbamoyl, sulfonamido and phenoxy, provided that R₆ and R₇ are not both hydrogen, and wherein R₈ is selected from the group consisting of C₃₋₁₀ cycloalkyl optionally substituted at the carbon position adjacent to the N atom of R₄′ with aryl or heteroaryl wherein said aryl is optionally substituted with halogen, heteroaryl, and heteroaryl or aryl wherein said heteroaryl or aryl is substituted with one or more substituents selected from the group consisting of halogen and C₁₋₄ alkyl;
R₂′ is selected from the group consisting of heterocyclyl, and, mono-substituted or disubstituted aryl and heterocyclyl, wherein at least one substituent of said aryl or heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one or more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁ are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
   or a pharmaceutical acceptable addition salt or a stereochemical isomeric form thereof or a N-oxide thereof or a solvate thereof or a prodrug thereof.

It is also an aspect of the present invention to provide pharmaceutical compositions comprising at least one novel pteridine derivative represented by any one of the structural formulae VI to XIV and defined as described herein.

Yet another aspect of the present invention relates to a method of treatment or prevention of an infection due to a virus from the Flaviviridae family, by administering to a patient in need thereof a therapeutically effective amount of a pteridine derivative according to any one of the structural formulae VI to XIV as described herein. More specifically, said infection may be caused by the Hepatitis C Virus (HCV). In particular, the administration may be oral. And said therapeutically effective amount ranges from about 0.1 mg to about 5 mg per day per kg bodyweight of said patient. Another aspect of the present invention is the use of a pteridine derivative represented by any one of structural formulae VI to XIV and defined as described herein, as a medicament, in particular such use in the manufacture of a medicament for the treatment or prevention of an infection due to a virus of the Flaviviridae family. In particular, said medicament is used for the treatment or prevention of an infection due to HCV. Also in particular, said medicament is administered orally and/or in a therapeutic dosage ranging from about 0.1 mg to about 5 mg per day per kg bodyweight of the patient to be treated.

Another aspect of the present invention relates to pteridine derivative represented by any one of the structural formulae VI to XIV and defined as described herein, for use in the treatment or prevention of an infection due to a virus from the Flaviviridae family, in particular due to HCV.

Another aspect of the present invention is compound 2-amino-4-isopropoxy-6-(4-fluorophenyl)-pteridine.

Also an aspect of the present invention is a method of treatment or prevention of an infection due to a virus from the Flaviridae family, by administering to a patient in need thereof a therapeutically effective amount of a pteridine derivative selected from the group consisting of 2-amino-4-ethoxy-6-(4-fluorophenyl)-pteridine and 2-amino-4-isopropoxy-6-(4-fluorophenyl)-pteridine.

Another aspect of the present invention comprises the compounds 2-amino-4-ethoxy-6-(4-fluorophenyl)-pteridine or 2-amino-4-isopropoxy-6-(4-fluorophenyl)-pteridine for use as a medicament.

A final aspect of the present invention is the use of 2-amino-4-ethoxy-6-(4-fluorophenyl)-pteridine or 2-amino-4-isopropoxy-6-(4-fluorophenyl)-pteridine in the preparation of a medicament for the treatment or prevention of an infection due to a virus from the Flaviridae family, in particular HCV.

### BRIEF DESCRIPTION OF THE DRAWINGS

FIG. 1 schematically shows a method for making pteridine derivatives according to the present invention wherein the substituent on position 2 of the pteridine core structure is an optionally further substituted alkyl amino (derived from an aldehyde), and the substituent on position 4 is alkoxy.

FIG. 2 schematically shows an alternative method for making pteridine derivatives according to the present invention wherein the substituent on position 4 of the pteridine core structure is derived from an amine (aliphatic or cyclic), and the substituent on position 4 is alkoxy.

### DEFINITIONS

Unless otherwise stated herein, the term “tri-substituted” means that three of the carbon atoms being in positions 2, 4 and 6 or, alternatively, in positions 2, 4 and 7 of the pteridine moiety (according to standard atom numbering for the pteridine moiety) are substituted with an atom or group of atoms other than hydrogen. The term “tetra-substituted” means that all four carbon atoms being in positions 2, 4, 6 and 7 of the pteridine moiety are substituted with an atom or group of atoms other than hydrogen.

As used herein with respect to a substituting radical, and unless otherwise stated, the term “C₁₋₇ alkyl” means straight and branched chain saturated acyclic hydrocarbon monovalent radicals having from 1 to 7 carbon atoms such as, for example, methyl, ethyl, propyl, n-butyl, 1-methylethyl (isopropyl), 2-methylpropyl (isobutyl), 1,1-dimethylethyl (ter-butyl), 2-methylbutyl, n-pentyl, dimethylpropyl, n-hexyl, 2-methylpentyl, 3-methylpentyl, n-heptyl and the like. By analogy, the term “C₁₋₁₂ alkyl” refers to such radicals having from 1 to 12 carbon atoms, i.e. up to and including dodecyl.

As used herein with respect to a substituting radical, and unless otherwise stated, the term “acyl” broadly refers to a substituent derived from an acid such as an organic monocarboxylic acid, a carbonic acid, a carbamic acid (resulting into a carbamoyl substituent) or the thioacid or imidic acid (resulting into a carbamidoyl substituent) corresponding to said acids, and the term “sulfonyl” refers to a substituent derived from an organic sulfonic acid, wherein said acids comprise an aliphatic, aromatic or heterocyclic group in the molecule. A more specific kind of “acyl” group within the scope of the above definition refers to a carbonyl (oxo) group adjacent to a C₁₋₇ alkyl, a C₃₋₁₀ cycloalkyl, an aryl, an arylalkyl or a heterocyclic group, all of them being such as herein defined. Suitable examples of acyl groups are to be found below.

Acyl and sulfonyl groups originating from aliphatic or cycloaliphatic monocarboxylic acids are designated herein as aliphatic or cycloaliphatic acyl and sulfonyl groups and include, but are not limited to, the following:
alkanoyl (for example formyl, acetyl, propionyl, butyryl, isobutyryl, valeryl, isovaleryl, pivaloyl and the like);
cycloalkanoyl (for example cyclobutanecarbonyl, cyclopentanecarbonyl, cyclo-hexanecarbonyl, 1-adamantanecarbonyl and the like);
cycloalkyl-alkanoyl (for example cyclohexylacetyl, cyclopentylacetyl and the like);
alkenoyl (for example acryloyl, methacryloyl, crotonoyl and the like);
alkylthioalkanoyl (for example methylthioacetyl, ethylthioacetyl and the like);
alkanesulfonyl (for example mesyl, ethanesulfonyl, propanesulfonyl and the like);
alkoxycarbonyl (for example methoxycarbonyl, ethoxycarbonyl, propoxycarbonyl, isopropoxycarbonyl, butoxycarbonyl, isobutoxycarbonyl and the like);
alkylcarbamoyl (for example methylcarbamoyl and the like);
(N-alkyl)-thiocarbamoyl (for example (N-methyl)-thiocarbamoyl and the like);
alkylcarbamidoyl (for example methylcarbamidoyl and the like); and
alkoxyalkyl (for example methoxyalkyl, ethoxyalkyl, propoxyalkyl and the like);

Acyl and sulfonyl groups may also originate from aromatic monocarboxylic acids and include, but are not limited to, the following:
aroyl (for example benzoyl, toluoyl, xyloyl, 1-naphthoyl, 2-naphthoyl and the like);
arylalkanoyl (for example phenylacetyl and the like);
arylalkenoyl (for example cinnamoyl and the like);
aryloxyalkanoyl (for example phenoxyacetyl and the like);
arylthioalkanoyl (for example phenylthioacetyl and the like);
arylaminoalkanoyl (for example N-phenylglycyl, and the like);
arylsulfonyl (for example benzenesulfonyl, toluenesulfonyl, naphthalene sulfonyl and the like);
aryloxycarbonyl (for example phenoxycarbonyl, naphthyloxycarbonyl and the like);
arylalkoxycarbonyl (for example benzyloxycarbonyl and the like);
arylcarbamoyl (for example phenylcarbamoyl, naphthylcarbamoyl and the like);
arylglyoxyloyl (for example phenylglyoxyloyl and the like).
arylthiocarbamoyl (for example phenylthiocarbamoyl and the like); and
arylcarbamidoyl (for example phenylcarbamidoyl and the like).

Acyl groups may also originate from an heterocyclic monocarboxylic acids and include, but are not limited to, the following:
heterocyclic-carbonyl, in which said heterocyclic group is as defined herein, preferably an aromatic or non-aromatic 5- to 7-membered heterocyclic ring with one or more heteroatoms selected from the group consisting of nitrogen, oxygen and sulfur in said ring (for example thiophenoyl, furoyl, pyrrolecarbonyl, nicotinoyl and the like); and
heterocyclic-alkanoyl in which said heterocyclic group is as defined herein, preferably an aromatic or non-aromatic 5- to 7-membered heterocyclic ring with one or more heteroatoms selected from the group consisting of nitrogen, oxygen and sulfur in said ring (for example thiophenylacetyl, furylacetyl, imidazolylpropionyl, tetrazolylacetyl, 2-(2-amino-4-thiazolyl)-2-methoxyiminoacetyl and the like).

As used herein with respect to a substituting radical, and unless otherwise stated, the term “C₁₋₇ alkylene” means the divalent hydrocarbon radical corresponding to the above defined C₁₋₇ alkyl, such as methylene, bis(methylene), tris(methylene), tetramethylene, hexamethylene and the like.

As used herein with respect to a substituting radical, and unless otherwise stated, the term “C₃₋₁₀ cycloalkyl” means a mono- or polycyclic saturated hydrocarbon monovalent radical having from 3 to 10 carbon atoms, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl.

As used herein with respect to a substituting radical, and unless otherwise stated, the term “C₃₋₁₀ cycloalkyl-alkyl” refers to an aliphatic saturated hydrocarbon monovalent radical (preferably a C₁₋₇ alkyl such as defined above) to which a C₃₋₁₀ cycloalkyl (such as defined above) is already linked such as, but not limited to, cyclohexylmethyl, cyclopentylmethyl and the like.

As used herein with respect to a substituting radical, and unless otherwise stated, the term “C₃₋₁₀ cycloalkylene” means the divalent hydrocarbon radical corresponding to the above defined C₃₋₁₀ cycloalkyl.

As used herein with respect to a substituting radical, and unless otherwise stated, the term “aryl” designate any mono- or polycyclic aromatic monovalent hydrocarbon radical having from 6 up to 30 carbon atoms such as but not limited to phenyl, naphthyl, anthracenyl, phenanthracyl, fluoranthenyl, chrysenyl, pyrenyl, biphenylyl, terphenyl, picenyl, indenyl, biphenyl, indacenyl, benzocyclobutenyl, benzocyclooctenyl and the like, including fused benzo-C₄₋₈ cycloalkyl radicals (the latter being as defined above) such as, for instance, indanyl, tetrahydronaphthyl, fluorenyl and the like, all of the said radicals being optionally substituted with one or more substituents independently selected from the group consisting of halogen, amino, trifluoromethyl, hydroxyl, sulfhydryl and nitro, such as for instance 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein, e.g. with respect to a substituting radical such as the combination of substituents in certain positions of the pteridine ring together with the carbon atoms in the same positions of said ring, and unless otherwise stated, the term “homocyclic” means a mono- or polycyclic, saturated or mono-unsaturated or polyunsaturated hydrocarbon radical having from 4 up to 15 carbon atoms but including no heteroatom in the said ring; for instance said combination of substituents may form a C₂₋₆ alkylene radical, such as tetramethylene, which cyclizes with the carbon atoms in certain positions of the pteridine ring.

As used herein with respect to a substituting radical (including the combination of substituents in certain positions of the pteridine ring together with the carbon atoms in the same positions of said ring), and unless otherwise stated, the term “heterocyclic” means a mono- or polycyclic, saturated or mono-unsaturated or polyunsaturated monovalent hydrocarbon radical having from 2 up to 15 carbon atoms and including one or more heteroatoms in one or more heterocyclic rings, each of said rings having from 3 to 10 atoms (and optionally further including one or more heteroatoms attached to one or more carbon atoms of said ring, for instance in the form of a carbonyl or thiocarbonyl or selenocarbonyl group, and/or to one or more heteroatoms of said ring, for instance in the form of a sulfone, sulfoxide, N-oxide, phosphate, phosphonate or selenium oxide group), each of said heteroatoms being independently selected from the group consisting of nitrogen, oxygen, sulfur, selenium and phosphorus, also including radicals wherein a heterocyclic ring is fused to one or more aromatic hydrocarbon rings for instance in the form of benzo-fused, dibenzo-fused and naphto-fused heterocyclic radicals; within this definition are included heterocyclic radicals such as, but not limited to, diazepinyl, oxadiazinyl, thiadiazinyl, dithiazinyl, triazolonyl, diazepinonyl, triazepinyl, triazepinonyl, tetrazepinonyl, benzoquinolinyl, benzothiazinyl, benzothiazinonyl, benzoxa-thiinyl, benzodioxinyl, benzodithiinyl, benzoxazepinyl, benzothiazepinyl, benzodiazepinyl, benzodioxepinyl, benzodithiepinyl, benzoxazocinyl, benzo-thiazocinyl, benzodiazocinyl, benzoxathiocinyl, benzodioxocinyl, benzotrioxepinyl, benzoxathiazepinyl, benzoxadiazepinyl, benzothia-diazepinyl, benzotriazepinyl, benzoxathiepinyl, benzotriazinonyl, benzoxazolinonyl, azetidinonyl, azaspiroundecyl, dithiaspirodecyl, selenazinyl, selenazolyl, selenophenyl, hypoxanthinyl, azahypo-xanthinyl, bipyrazinyl, bipyridinyl, oxazolidinyl, diselenopyrimidinyl, benzodioxocinyl, benzopyrenyl, benzopyranonyl, benzophenazinyl, benzoquinolizinyl, dibenzo-carbazolyl, dibenzoacridinyl, dibenzophenazinyl, dibenzothiepinyl, dibenzoxepinyl, dibenzopyranonyl, dibenzoquinoxalinyl, dibenzothiazepinyl, dibenzisoquinolinyl, tetraazaadamantyl, thiatetraazaadamantyl, oxauracil, oxazinyl, dibenzothiophenyl, dibenzofuranyl, oxazolinyl, oxazolonyl, azaindolyl, azolonyl, thiazolinyl, thiazolonyl, thiazolidinyl, thiazanyl, pyrimidonyl, thiopyrimidonyl, thiamorpholinyl, azlactonyl, naphtindazolyl, naphtindolyl, naphtothiazolyl, naphtothioxolyl, naphtoxindolyl, naphto-triazolyl, naphtopyranyl, oxabicycloheptyl, azabenzimidazolyl, azacycloheptyl, azacyclooctyl, azacyclononyl, azabicyclononyl, tetrahydrofuryl, tetrahydropyranyl, tetrahydro-pyronyl, tetrahydroquinoleinyl, tetrahydrothienyl and dioxide thereof, dihydrothienyl dioxide, dioxindolyl, dioxinyl, dioxenyl, dioxazinyl, thioxanyl, thioxolyl, thiourazolyl, thiotriazolyl, thiopyranyl, thiopyronyl, coumarinyl, quinoleinyl, oxyquinoleinyl, quinuclidinyl, xanthinyl, dihydropyranyl, benzodihydrofuryl, benzothiopyronyl, benzothiopyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl (benzothiofuranyl), phenopyronyl, phenoxazolyl, pyridinyl, dihydropyridinyl, tetrahydropyridinyl, piperidinyl, morpholinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofuryl, furoyl, hydantoinyl, dioxolanyl, dioxolyl, dithianyl, dithienyl, dithiinyl, thienyl, indolyl, indazolyl, benzofuryl, quinolyl, quinazolinyl, quinoxalinyl, carbazolyl, phenoxazinyl, phenothiazinyl, xanthenyl, purinyl, benzothienyl, naphtothienyl, thianthrenyl, pyranyl, pyronyl, benzopyronyl, isobenzofuranyl, chromenyl, phenoxathiinyl, indolizinyl, quinolizinyl, isoquinolyl, phthalazinyl, naphthiridinyl, cinnolinyl, pteridinyl, carbolinyl, acridinyl, perimidinyl, phenanthrolinyl, phenazinyl, phenothiazinyl, imidazolinyl, imidazolidinyl, benzimi-dazolyl, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, oxetanonyl, homopiperazinyl, homopiperidinyl, thietyl, thietanyl, diazabicyclooctyl, diazetyl, diaziridinonyl, diaziridinethionyl, chromanyl, chromanonyl, thiochromanyl, thiochromanonyl, thiochromenyl, benzofuranyl, benzisothiazolyl, benzocarbazolyl, benzochromonyl, benzisoalloxazinyl, benzocoumarinyl, thiocoumarinyl, pheno-metoxazinyl, phenoparoxazinyl, phentriazinyl, thiodiazinyl, thiodiazolyl, indoxyl, thioindoxyl, benzodiazinyl (e.g. phtalazinyl), phtalidyl, phtalimidinyl, phtalazonyl, alloxazinyl, dibenzopyronyl (i.e. xanthonyl), xanthionyl, isatyl, isopyrazolyl, isopyrazolonyl, urazolyl, urazinyl, uretinyl, uretidinyl, succinyl, succinimido, benzylsultimyl, benzylsultamyl and the like, including all possible isomeric forms thereof, wherein each carbon atom of said heterocyclic ring may furthermore be independently substituted with a substituent selected from the group consisting of halogen, nitro, C₁₋₇ alkyl (optionally containing one or more functions or radicals selected from the group consisting of carbonyl (oxo), alcohol (hydroxyl), ether (alkoxy), acetal, amino, imino, oximino, alkyloximino, amino-acid, cyano, carboxylic acid ester or amide, nitro, thio C₁₋₇ alkyl, thio C₃₋₁₀ cycloalkyl, C₁₋₇ alkylamino, cycloalkylamino, alkenylamino, cycloalkenylamino, alkynylamino, arylamino, arylalkyl-amino, hydroxylalkylamino, mercaptoalkylamino, heterocyclic-substituted alkylamino, heterocyclic amino, heterocyclic-substituted arylamino, hydrazino, alkylhydrazino, phenylhydrazino, sulfonyl, sulfonamido and halogen), C₃₋₇ alkenyl, C₂₋₇ alkynyl, halo C₁₋₇ alkyl, C₃₋₁₀ cycloalkyl, aryl, arylalkyl, alkylaryl, alkylacyl, arylacyl, hydroxyl, amino, C₁₋₇ alkylamino, cycloalkylamino, alkenylamino, cycloalkenylamino, alkynylamino, arylamino, arylalkylamino, hydroxyalkylamino, mercaptoalkylamino, heterocyclic-substituted alkylamino, heterocyclic amino, heterocyclic-substituted arylamino, hydrazino, alkylhydrazino, phenylhydrazino, sulfhydryl, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxylamino, cyano, carboxylic acid or esters or thioesters or amides thereof, thiocarboxylic acid or esters or thioesters or amides thereof; depending upon the number of unsaturations in the 3 to 10 atoms ring, heterocyclic radicals may be sub-divided into heteroaromatic (or “heteroaryl”) radicals and non-aromatic heterocyclic radicals; when a heteroatom of said non-aromatic heterocyclic radical is nitrogen, the latter may be substituted with a substituent selected from the group consisting of C₁₋₇ alkyl, C₃₋₁₀ cycloalkyl, aryl, arylalkyl and alkylaryl.

As used herein with respect to a substituting radical, and unless otherwise stated, the terms “C₁₋₇ alkoxy”, “C₃₋₁₀ cycloalkoxy”, “aryloxy”, “arylalkoxy”, “oxyheterocyclic”, “heterocyclic-substituted alkoxy”, “thio C₁₋₇ alkyl”, “thio C₃₋₁₀ cycloalkyl”, “arylthio”, “arylalkylthio” and “thioheterocyclic” refer to substituents wherein a carbon atom of a C₁₋₇ alkyl, respectively a C₃₋₁₀ cycloalkyl, aryl, arylalkyl, heterocyclic radical or heterocyclic-substituted alkyl (each of them such as defined herein), is attached to an oxygen atom or a divalent sulfur atom through a single bond such as, but not limited to, methoxy, ethoxy, propoxy, butoxy, pentoxy, isopropoxy, sec-butoxy, tert-butoxy, isopentoxy, cyclopropyloxy, cyclobutyloxy, cyclopentyloxy, thiomethyl, thioethyl, thiopropyl, thiobutyl, thiopentyl, thiocyclopropyl, thiocyclobutyl, thiocyclopentyl, thiophenyl, phenyloxy, benzyloxy, mercaptobenzyl and cresoxy, and various isomers of piperidinoxy, 1-methylpiperidinoxy, pyrrolidinoxy, pyridinoxy, tetrahydrofuranyloxy, morpholinoethoxy, piperazinoethoxy, piperi-dinoethoxy, pyridinoethoxy, pyrrolidinoethoxy, piperidinomethoxy, methylpyridinoxy, methylquinolinoxy, pyridinopropoxy and the like.

As used herein with respect to a substituting atom, and unless otherwise stated, the term halogen means any atom selected from the group consisting of fluorine, chlorine, bromine and iodine.

As used herein with respect to a substituting radical, and unless otherwise stated, the term “halo C₁₋₇ alkyl” means a C₁₋₇ alkyl radical (such as above defined) in which one or more hydrogen atoms are independently replaced by one or more halogens (preferably fluorine, chlorine or bromine), such as but not limited to difluoromethyl, trifluoromethyl, trifluoroethyl, octafluoropentyl, dodecafluoroheptyl, dichloromethyl and the like.

As used herein with respect to a substituting radical, and unless otherwise stated, the terms “C₂₋₇ alkenyl” designate a straight and branched acyclic hydrocarbon monovalent radical having one or more ethylenic unsaturations and having from 2 to 7 carbon atoms such as, for example, vinyl, 1-propenyl, 2-propenyl (allyl), 1-butenyl, 2-butenyl, 2-pentenyl, 3-pentenyl, 3-methyl-2-butenyl, 3-hexenyl, 2-hexenyl, 2-heptenyl, 1,3-butadienyl, pentadienyl, hexadienyl, heptadienyl, heptatrienyl and the like, including all possible isomers thereof.

As used herein with respect to a substituting radical, and unless otherwise stated, the term “C₃₋₁₀ cycloalkenyl” means a monocyclic mono- or polyunsaturated hydrocarbon monovalent radical having from 3 to 8 carbon atoms, such as for instance cyclopropenyl, cyclobutenyl, cyclopentenyl, cyclopentadienyl, cyclohexenyl, cyclohexadienyl, cycloheptenyl, cyclohepta-dienyl, cycloheptatrienyl, cyclooctenyl, cyclooctadienyl and the like, or a C₇₋₁₀ polycyclic mono- or polyunsaturated hydrocarbon mono-valent radical having from 7 to 10 carbon atoms such as dicyclopentadienyl, fenchenyl (including all isomers thereof, such as α-pinolenyl), bicyclo[2.2.1]hept-2-enyl, bicyclo[2.2.1]hepta-2,5-dienyl, cyclo-fenchenyl and the like.

As used herein with respect to a substituting radical, and unless otherwise stated, the term “C₂₋₇ alkynyl” defines straight and branched chain hydrocarbon radicals containing one or more triple bonds and optionally at least one double bond and having from 2 to 7 carbon atoms such as, for example, acetylenyl, 1-propynyl, 2-propynyl, 1-butynyl, 2-butynyl, 2-pentynyl, 1-pentynyl, 3-methyl-2-butynyl, 3-hexynyl, 2-hexynyl, 1-penten-4-ynyl, 3-penten-1-ynyl, 1,3-hexadien-1-ynyl and the like.

As used herein with respect to a substituting radical, and unless otherwise stated, the terms “arylalkyl”, “arylalkenyl” and “heterocyclic-substituted alkyl” refer to an aliphatic saturated or ethylenically unsaturated hydrocarbon monovalent radical (preferably a C₁₋₇ alkyl or C₂₋₇ alkenyl radical such as defined above) onto which an aryl or heterocyclic radical (such as defined above) is already bonded via a carbon atom, and wherein the said aliphatic radical and/or the said aryl or heterocyclic radical may be optionally substituted with one or more substituents independently selected from the group consisting of halogen, amino, hydroxyl, sulfhydryl, C₁₋₇ alkyl, C₁₋₇ alkoxy, trifluoromethyl and nitro, such as but not limited to benzyl, 4-chlorobenzyl, 4-fluorobenzyl, 2-fluorobenzyl, 3,4-dichlorobenzyl, 2,6-dichlorobenzyl, 3-methylbenzyl, 4-methylbenzyl, 4-ter-butylbenzyl, phenylpropyl, 1-naphthylmethyl, phenylethyl, 1-amino-2-phenylethyl, 1-amino-2-[4-hydroxy-phenyl]ethyl, 1-amino-2-[indol-2-yl]ethyl, styryl, pyridylmethyl (including all isomers thereof), pyridylethyl, 2-(2-pyridyl)isopropyl, oxazolylbutyl, 2-thienylmethyl, pyrrolylethyl, morpholinylethyl, imidazol-1-yl-ethyl, benzodioxolylmethyl and 2-furylmethyl.

As used herein with respect to a substituting radical, and unless otherwise stated, the terms “alkylaryl” and “alkyl-substituted heterocyclic” refer to an aryl or, respectively, heterocyclic radical (such as defined above) onto which are bonded one or more aliphatic saturated or unsaturated hydrocarbon monovalent radicals, preferably one or more C₁₋₇ alkyl, C₂₋₇ alkenyl or C₃₋₁₀ cycloalkyl radicals as defined above such as, but not limited to, o-toluoyl, m-toluoyl, p-toluoyl, 2,3-xylyl, 2,4-xylyl, 3,4-xylyl, o-cumenyl, m-cumenyl, p-cumenyl, o-cymenyl, m-cymenyl, p-cymenyl, mesityl, ter-butylphenyl, lutidinyl (i.e. dimethylpyridyl), 2-methylaziridinyl, methyl-benzimidazolyl, methylbenzofuranyl, methylbenzothiazolyl, methylbenzotriazolyl, methylbenzoxazolyl and methylbenzselenazolyl.

As used herein with respect to a substituting radical, and unless otherwise stated, the term “alkoxyaryl” refers to an aryl radical (such as defined above) onto which is (are) bonded one or more C₁₋₇ alkoxy radicals as defined above, preferably one or more methoxy radicals, such as, but not limited to, 2-methoxyphenyl, 3-methoxyphenyl, 4-methoxyphenyl, 3,4-dimethoxyphenyl, 2,4,6-trimethoxyphenyl, methoxynaphtyl and the like.

As used herein with respect to a substituting radical, and unless otherwise stated, the terms “alkylamino”, “cycloalkylamino”, “alkenylamino”, “cyclo-alkenylamino”, “arylamino”, “arylalkylamino”, “heterocyclic-substituted alkyl-amino”, “heterocyclic-substituted arylamino”, “heterocyclic amino”, “hydroxy-alkylamino”, “mercaptoalkylamino” and “alkynylamino” mean that respectively one (thus monosubstituted amino) or even two (thus disubstituted amino) C₁₋₇ alkyl, C₃₋₁₀ cycloalkyl, C₂₋₇ alkenyl, C₃₋₁₀ cycloalkenyl, aryl, arylalkyl, heterocyclic-substituted alkyl, heterocyclic-substituted aryl, heterocyclic (provided in this case the nitrogen atom is attached to a carbon atom of the heterocyclic ring), mono- or polyhydroxy C₁₋₇ alkyl, mono- or polymercapto C₁₋₇ alkyl, or C₂₋₇ alkynyl radical(s) (each of them as defined herein, respectively, and including the presence of optional substituents independently selected from the group consisting of halogen, amino, hydroxyl, sulfhydryl, C₁₋₇ alkyl, C₁₋₇ alkoxy, trifluoromethyl and nitro) is/are attached to a nitrogen atom through a single bond such as, but not limited to, anilino, 2-bromoanilino, 4-bromoanilino, 2-chloroanilino, 3-chloroanilino, 4-chloroanilino, 3-chloro-4-methoxyanilino, 5-chloro-2-methoxyanilino, 2,3-dimethylanilino, 2,4-dimethylanilino, 2,5-dimethylanilino, 2,6-dimethylanilino, 3,4-dimethylanilino, 2-fluoroanilino, 3-fluoroanilino, 4-fluoroanilino, 3-fluoro-2-methoxyanilino, 3-fluoro-4-methoxyanilino, 2-fluoro-4-methylanilino, 2-fluoro-5-methylanilino, 3-fluoro-2-methylanilino, 3-fluoro-4-methylanilino, 4-fluoro-2-methylanilino, 5-fluoro-2-methylanilino, 2-iodoanilino, 3-iodoanilino, 4-iodoanilino, 2-methoxy-5-methylanilino, 4-methoxy-2-methylanilino, 5-methoxy-2-methylanilino, 2-ethoxyanilino, 3-ethoxy-anilino, 4-ethoxyanilino, benzylamino, 2-methoxybenzylamino, 3-methoxybenzyl-amino, 4-methoxybenzylamino, 2-fluorobenzylamino, 3-fluorobenzylamino, 4-fluoro-benzylamino, 2-chlorobenzylamino, 3-chlorobenzylamino, 4-chlorobenzylamino, 2-aminobenzylamino, diphenylmethylamino, α-naphthylamino, methylamino, dimethylamino, ethylamino, diethylamino, isopropylamino, propenylamino, n-butylamino, ter-butylamino, dibutylamino, 1,2-diaminopropyl, 1,3-diaminopropyl, 1,4-diaminobutyl, 1,5-diaminopentyl, 1,6-diaminohexyl, morpholinomethylamino, 4-morpholinoanilino, hydroxymethylamino, β-hydroxyethylamino and ethynylamino; this definition also includes mixed disubstituted amino radicals wherein the nitrogen atom is attached to two such radicals belonging to two different sub-sets of radicals, e.g. an alkyl radical and an alkenyl radical, or to two different radicals within the same sub-set of radicals, e.g. methylethylamino; among di-substituted amino radicals, symmetrically-substituted amino radicals are more easily accessible and thus usually preferred from a standpoint of ease of preparation.

As used herein with respect to a substituting radical, and unless otherwise stated, the terms “(thio)carboxylic acid ester”, “(thio)carboxylic acid thioester” and “(thio)carboxylic acid amide” refer to radicals wherein the carboxyl or thiocarboxyl group is bonded to the hydrocarbonyl residue of an alcohol, a thiol, a polyol, a phenol, a thiophenol, a primary or secondary amine, a polyamine, an amino-alcohol or ammonia, the said hydrocarbonyl residue being selected from the group consisting of alkyl, alkenyl, alkynyl, cycloalkyl, cycloalkenyl, aryl, arylalkyl, alkylaryl, alkylamino, cycloalkylamino, alkenylamino, cycloalkenylamino, arylamino, arylalkylamino, heterocyclic-substituted alkylamino, heterocyclic amino, heterocyclic-substituted arylamino, hydroxyalkylamino, mercapto-alkylamino or alkynylamino (such as above defined, respectively).

As used herein with respect to a substituting radical, and unless otherwise stated, the term “amino-acid” refers to a radical derived from a molecule having the chemical formula H₂N—CHR—COOH, wherein R is the side group of atoms characterising the amino-acid type; said molecule may be one of the 20 naturally-occurring amino-acids or any similar non naturally-occurring amino-acid.

As used herein and unless otherwise stated, the term “stereoisomer” refers to all possible different isomeric as well as conformational forms which the anti-viral active agents of the present invention may possess, in particular all possible stereochemically and conformationally isomeric forms, all diastereomers, enantiomers and/or conformers of the basic molecular structure. Some anti-viral active agents of the present invention may exist in different tautomeric forms, all of the latter being included within the scope of the present invention.

As used herein and unless otherwise stated, the term “enantiomer” means each individual optically active form of an anti-viral active agent of the present invention, having an optical purity or enantiomeric excess (as determined by methods standard in the art) of at least 80% (i.e. at least 90% of one enantiomer and at most 10% of the other enantiomer), preferably at least 90% and more preferably at least 98%.

As used herein and unless otherwise stated, the term “solvate” includes any combination which may be formed by an anti-viral active agent, i.e. a pteridine derivative of the present invention with a suitable inorganic solvent (e.g. hydrates) or organic solvent, such as but not limited to alcohols, ketones, esters, ethers, nitriles and the like.

As used herein and unless otherwise stated, the term “pro-drug” relates to certain precursor forms of the anti-viral active agents of the present invention. It may be desirable to formulate the pteridine derivatives of the present invention in the form of a chemical species which itself is not significantly biologically-active against a virus from the Flaviridae family, but which when delivered to the body of a human being or higher mammal will undergo a chemical reaction catalyzed by the normal function of the body, inter alia, enzymes present in the stomach or in blood serum, said chemical reaction having the effect of releasing a pteridine compound as defined herein-above. The term “pro-drug” thus conventionally relates to these species which may be converted in vivo into the anti-viral active pharmaceutical ingredient. The pro-drugs of the present invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used.

For the purposes of the present invention the term “therapeutically suitable pro-drug” is defined herein as a compound modified in such a way as to be transformed in vivo into the therapeutically active form, whether by way of a single or by multiple biological transformations, when in contact with the tissues of humans or mammals to which the pro-drug has been administered, and without undue toxicity, irritation, or allergic response, and achieving the intended therapeutic outcome.

The therapeutically effective amount to be administered according to the method of treatment or prevention of the present invention is usually in the range of about 0.01 mg to 20 mg, preferably about 0.1 mg to 5 mg, per day per kg bodyweight for human beings. Depending upon the severity of the pathologic condition to be treated and the patient's general condition, the said therapeutically effective amount may be divided into several sub-units per day, or may be administered at more than one day intervals. The patient to be treated may be any warm-blooded animal, preferably a mammal, more preferably a human being, suffering from an infection by a virus being a member of the Flaviridae family.

For most modes of administration, especially for the preferred oral mode of administration, it is preferred to formulate the pteridine anti-viral active agent of this invention together with one or more suitable pharmaceutically acceptable carriers or excipients.

The term “pharmaceutically acceptable carrier or excipient” as used herein in relation to pharmaceutical compositions for administration to a patient in need thereof means any material or substance with which the active principle, i.e. the pteridine derivative as defined herein-above, may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the antiviral agent containing compositions of this invention can suitably be used as concentrates, emulsions, solutions, granulates, dusts, sprays, aerosols, pellets or powders.

Suitable pharmaceutical carriers for use in the said pharmaceutical compositions and their formulation are well known to those skilled in the art. There is no particular restriction to their selection within the present invention although, due to the usually low or very low water-solubility of the pteridine active agents of this invention, special attention will be paid to the selection of suitable carrier combinations that can assist in properly formulating them in view of the expected time release profile. Suitable pharmaceutical carriers include additives such as, but not limited to, wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with conventional pharmaceutical practice, i.e. carriers and additives which do not create permanent damage to mammals. The medicaments of the present invention may be prepared in any known manner, for instance by homogeneously mixing, dissolving, spray-drying, coating and/or grinding the active ingredient, in a one-step or a multi-steps procedure, together with the one or more selected carrier materials and, where appropriate, the other additives such as, but not limited to, surface-active agents. These medicaments may also be prepared by micronisation, for instance in view to obtain them in the form of microspheres usually having a diameter of about 1 to 10 μm, namely for the manufacture of microcapsules for controlled or sustained release of the antiviral active ingredient.

Suitable surface-active agents which may be used in making the medicaments of the present invention include, but are not limited to, non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphthalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose include, but are not limited to, the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanyl-phosphatidylcholine, dipalmitoylphoshatidylcholine and their mixtures in various proportions.

Suitable non-ionic surfactants include, but are not limited to, poly-ethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates such as, but not limited to, polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants include, but are not limited to, water-soluble adducts of polyethylene oxide with poylypropylene glycol, ethylenediaminopolypropylene glycol containing from 1 to 10 carbon atoms in the alkyl chain, which adducts contain from 20 to 250 ethyleneglycol ether groups and/or from 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethylene-glycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include, but are not limited to, quaternary ammonium salts, preferably halides, having 4 hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, non substituted or halogenated lower alkyl, benzyl and/or hydroxy-lower alkyl radicals.

A more detailed description of surface-active agents suitable for the purpose of formulating the antiviral pteridine agents of this invention may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopaedia of Surfactants (Chemical Publishing Co., New York, 1981).

Structure-forming, thickening or gel-forming agents may also be included into the medicaments for the method of treatment or prevention of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Gelling agents which may be included into the medicaments for the method of treatment or prevention of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof. Gelatin and modified celluloses represent a preferred class of gelling agents.

Other optional excipients which may be included in the medicaments for the method of treatment or prevention of the present invention include, but are not limited to, additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof.

Additional ingredients may be included in order to control the duration of action of the biologically-active ingredient in the medicaments of the present invention. Control release compositions may thus be achieved by selecting appropriate polymer carriers such as for example polyesters, polyamino-acids, polyvinyl-pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems like liposomes, microspheres, microemulsions, nanoparticles, nanocapsules and so on. Depending on the route of administration, the medicament of the present invention may also require protective coatings.

Pharmaceutical forms suitable for injectable use include sterile aqueous solutions or dispersions and sterile powders for the extemporaneous preparation thereof. Typical carriers for this purpose therefore include, but are not limited to, biocompatible aqueous buffers, ethanol, glycerol, propylene glycol, polyethylene glycol, complexing agents such as cyclodextrins and the like, and mixtures thereof.

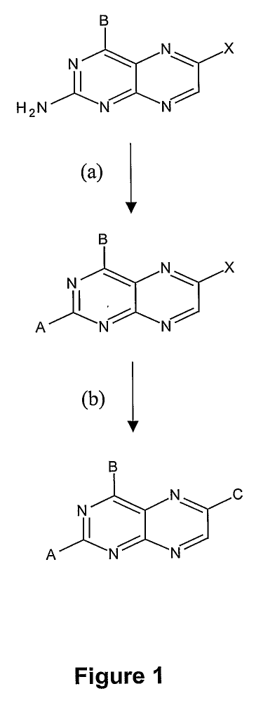

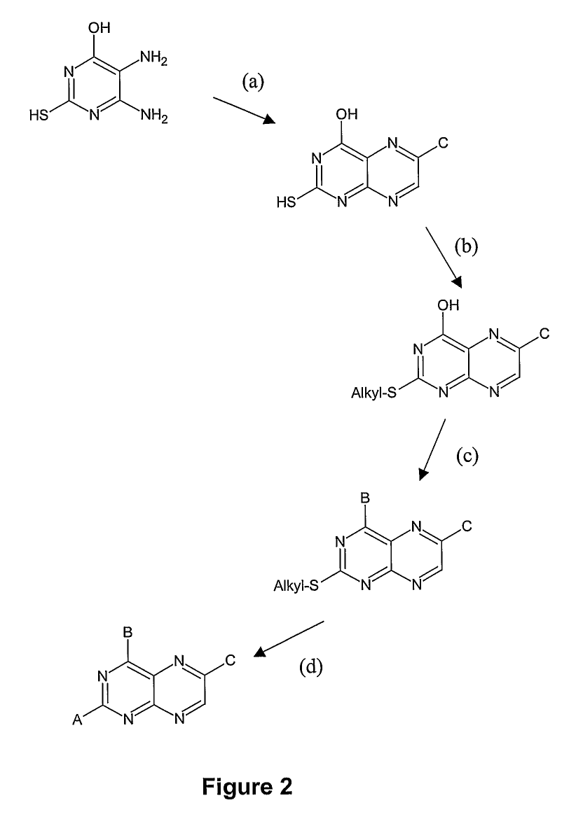

Synthesis and characterization of part of the 2,4,6-trisubstituted pteridine compounds for which anti-HCV activity has been tested and is described in Tables 2 and 3 below, has already been reported in U.S. Patent Application Publication No.'s 2004/0077859 and 2007/0032477, and in WO 2005/021003 respectively. Synthesis and characterization of most 2,4,6-trisubstituted pteridine compounds for which anti-HCV activity has been tested and is described in Table 1 below has already been reported in WO 2005/039587. As a complement to the above referred disclosure, synthesis and characterization of a few other 2,4,6-trisubstituted pteridines, for which anti-HCV activity has been tested and is reported in Table 1, is described below, as well as alternative methods of manufacture developed by the present inventors which may be used alternatively to, or may be combined with, the methods of synthesis already known in the art of pteridine derivatives. Depending upon the targeted final compound, methods of manufacture as described herein and illustrated by FIGS. 1 and 2, may be used as such or individual synthetic steps may be combined with methods of manufacture (or individual steps thereof) described in U.S. Patent Application Publication No.'s 2004/0077859 and 2007/0032477, and in WO 2005/021003. The methods of manufacture or individual synthetic steps described herein, are in particular usefull for the introduction of alternative substituents on positions 2 and 6 of the pteridine core structure, namely for those substituents as defined for formulae VI to XIV.

FIG. 1 schematically shows an alternative method for making pteridine derivatives according to the present invention wherein the substituent on position 2 of the pteridine core structure is an optionally further substituted alkyl amino (derived from an aldehyde), and the substituent on position 4 is (optionally substituted) alkoxy. In step (a), treatment of a 2-amino-6-halo-4-substituted-pteridine (X is preferably chlorine (described in WO 2005/025574), bromine or iodine) with an aldehyde under reductive amination conditions, such as those generated by the addition of sodium triacetoxyborohydride in the presence of an acid such as trifluoroacetic acid in an inert solvent such as isopropyl acetate. The synthetic step provides for the generation of a substituted amino substituent at the 2-position of the pteridine, such as those described for the novel compounds herein disclosed. For example, reaction with an aldehyde according to the formula O═CHR₆R₇ wherein R₆ and R₇ are as defined for structural formulae VI, VIII, X, XI, XIII and XIV. In particular, optionally mono-substituted or di-substituted benzaldehydes suitable as starting materials for the reaction of step (a) of the method illustrated by FIG. 1 include, but are not limited to, benzaldehyde, salicylaldehyde, o-tolualdehyde, m-tolualdehyde, p-tolualdehyde, o-anisaldehyde, m-anisaldehyde, p-anisaldehyde, 2,5-dihydroxybenzaldehyde, 4-propoxy-benzaldehyde, 4-phenoxy-benzaldehyde, 3-(3,4-dichlorophenoxy)benzaldehyde, 3-(3,5-dichlorophenoxy)benzaldehyde, 2-bromo-benzaldehyde, 3-bromobenzaldehyde, 4-bromo-benzaldehyde, 2-chloro-benzaldehyde, 3-chlorobenzaldehyde, 4-chlorobenzaldehyde, 2-fluorobenzaldehyde, 3-fluoro-benzaldehyde, 4-fluorobenzaldehyde, 2,3-dichlorobenzaldehyde, 2,4-dichloro-benzaldehyde, 2,6-dichlorobenzaldehyde, 3,4-dichlorobenzaldehyde, 3,5-dichloro-benzaldehyde, 2,3-difluoro-benzaldehyde, 2,4-difluorobenzaldehyde, 2,5-difluoro-benzaldehyde, 2,6-difluorobenzaldehyde, 3,4-difluorobenzaldehyde, 3,5-difluoro-benzaldehyde, 2,3,4-trifluorobenzaldehyde, 2-(trifluoromethyl)benzaldehyde, 3-(trifluoro-methyl)benzaldehyde, 4-(trifluoromethyl)benzaldehyde, 3-(trifluoro-methoxy)benzaldehyde, 5-(trifluoromethoxy)salicylaldehyde, 3,5-dichloro-salicyl-aldehyde, 2-nitrobenzaldehyde, 3-nitrobenzaldehyde, 4-nitrobenzaldehyde, 3-cyanobenzaldehyde, 4-cyano-benzaldehyde, 4-dimethylamino-1-naphthaldehyde, 4-(dimethylamino)benzaldehyde, 4-(diethylamino)benzaldehyde and 3,4,5-trimethoxybenzaldehyde.

Palladium-mediated aryl-aryl cross coupling (Suzuki type) occurs in step (b) by treating the 6-haloquinazoline with aryl boronic acid or boronic acid esters in the presence of aqueous base and a palladium(0) catalyst such as Pd(PPh₃)₄ to give the desired 6-arylpteridines.

Most of the available methods make use of a boronic acid, or a pinacol ester thereof, for introducing substituent at position 6 of the pteridine core structure. Suitable aryl-boronic acids, in particular to obtain pteridine derivatives represented by structural formulae VI to XIV as described herein, include, but are not limited to, the following commercially available materials wherein the aryl group is 3-acetamidophenyl, 4-acetamindophenyl, 4-acetylphenyl, 3-acetylphenyl, 2-acetylphenyl, 5-acetyl-2-chlorophenyl, 4-acetyl-3-fluorophenyl, 5-acetyl-2-fluorophenyl, 3-aminophenyl, 4-aminomethylphenyl, 3-aminophenyl, 4-benzyloxybenzene, 3-benzyloxybenzene, 4-benzyloxy-2-fluorophenyl, 4-benzyloxy-3-fluorophenyl, biphenyl-3-, 3,5-bis(trifluoromethyl)benzene, 4-bromophenyl, 3-bromophenyl, 4-bromo-2,5-dimethylphenyl, 2-bromo-5-fluorophenyl, 2-Bromo-6-fluorophenyl, 4-carboxyphenyl, 2-carboxyphenyl, 2-carboxy-5-fluorophenyl, 4-carboxy-2-chlorophenyl, 5-carboxy-2-chlorophenyl, 4-carboxy-3-chlorophenyl, 3-carboxyphenyl, 2-chloro-5-formylphenyl, 2-chloro-5-hydroxyphenyl, 3-chloro-4-fluorophenyl, 2-chloro-4-fluorophenyl, 4-chloro-2-fluorophenyl, 3-chloro-5-methoxyphenyl, 2-chloro-4-methylphenyl, 2-chloro-5-methylphenyl, 2-chloro-5-trifluoromethoxyphenyl, 3-chloro-5-trifluoromethylphenyl, 4-chloro-2-trifluoromethylphenyl, 4-chlorophenyl, 3-chlorophenyl, 2-chlorophenyl, 4-cyanophenyl, 3-cyanophenyl, 2-cyanophenyl, 3,5-dibromophenyl, 2,6-dichlorophenyl, 3,4-dichlorophenyl, 2,4-dichlorophenyl, 2,3-dichlorophenyl, 3,5-dichlorophenyl, 3,5-difluorophenyl, 3,5-difluoro-2-methoxyphenyl, 3,4-difluorophenyl, 2,6-difluorophenyl, 2,5-difluorophenyl, 2,4-difluorophenyl, 2,3-difluorophenyl, 2,3-dihydro-1,4-benzodioin-6-yl, 2,4-dimethoxybenzene, 4-(N,N-dimethylamino)phenyl, 2-(N,N-dimethylaminomethyl)phenyl, 3,5-dimethylphenyl, 3,4-dimethylphenyl, 2,6-dimethylphenyl, 2,6-dimethoxyphenyl, 2,5-dimethoxyphenyl, 2,4-dimethoxyphenyl, 4-ethoxyphenyl, 2-ethoxyphenyl, 4-ethoxycarbonylphenyl, 3-ethoxycarbonylphenyl, 4-ethylphenyl, 4-fluorophenyl, 3-fluorophenyl, 2-fluorophenyl, 3-fluoro-4-formylphenyl, 4-fluoro-2-methylphenyl, 2-fluoro-5-methylphenyl, 4-fluoro-3-formylphenyl, 2-fluoro-5-methoxyphenyl, 5-fluoro-2-methoxycarbonylphenyl, 2-formyl-5-methoxyphenyl, 5-formyl-2-methoxyphenyl, 2-formyl-5-methylphenyl, 4-formylphenyl, 3-formylphenyl, 2-formylphenyl, 3-hydroxy-4-methoxycarbonylphenyl, 4-(hydroxymethyl)phenyl, 3-(hydroxymethyl)phenyl, 4-hydroxyphenyl, 3-hydroxyphenyl, 4-iodophenyl, 3-iodophenyl, 3-isopropoxycarbonylphenyl, 4-isopropoxycarbonylphenyl, 4-methanesulfonylphenyl, 2-methoxy-5-formylphenyl, 5-methoxy-2-formylphenyl, 4-methoxy-2-formylphenyl, 4-methoxycarbonylphenyl, 3-methoxycarbonylphenyl, 4-methoxyphenyl, 3-methoxyphenyl, 2-methoxyphenyl, 3,4-methylenedioxyphenyl, 4-methylphenyl, 2-methylphenyl, 4-(methylthio)phenyl, 3-(methylthio)phenyl, 4-morpholinophenyl, 3-nitrophenyl, 4-phenoxyphenyl, 4-(tert-butoxycarbonylamino)-3-methoxyphenyl, 2-(tert-butoxycarbonyl)phenyl, 3-(tert-butoxycarbonyl)phenyl, 4-(tert-butoxycarbonyl)phenyl, 4-tert-butylphenyl, 4-(tetrahdro-2H-pyran-2-yloxy)phenyl, 4-(2-thienyl)phenyl, trans-β-styrene, 4-tolyl, 3-tolyl, 2-tolyl, 4-trifluoromethoxyphenyl, 4-(trimethylammonium)methylphenyl, 2,4,6-trimethylphenyl, 3,4,5-trifluorophenyl, 3-trifluoromethylphenyl, 4-trifluoromethoxyphenyl, 3-trifluoromethoxyphenyl, 3-trifluoromethylphenyl, 2-trifluoromethylphenyl, 3,4,5-trimethoxyphenyl, 4-vinylphenyl, 6-benzyloxy-2-naphthyl, 1-naphthalene, 2-naphthalene, or 1-biphenylenyl.

Suitable heterocyclic-boronic acids in particular to obtain pteridine derivatives represented by structural formulae VI to XIV as described herein, include, but are not limited to, the following commercially available materials wherein the heterocyclic group is 2-acetamidopyridin-5-yl, 2-benzothienyl, 1-benzothiophen-3-yl, 1-benzothiophen-2-yl, 2-bromo-3-chloropyridin-4-yl, 5-bromo-2,3-dihydrobenzo[b]furan-7-yl, 2-bromo-3-methylpyridin-5-yl, 2-bromopyridin-5-yl, 5-bromothien-2-yl, 2-chloro-6-isopropylpyridin-3-yl, 2-chloro-3-methylpyridin-5-yl, 5-chlorothien-2-yl, dibenzo[b,d]furan-4-yl, 2-chloro-3-fluoropyridin-4-yl, dibenzo[b,d]thien-4-yl, 3,4-dihydro-2H-1,5-benzodioxepin-7-yl, 2,5-dibromo-3-pyridinyl, 2,6-dichloro-pyridin-3-yl, 2,3-dihydro-1-benzofuran-5-yl, 2,4-dimethoxypyrimidin-5-yl, 3,5-dimethylisoxazol-4-yl, 1-[1,3]dioxolan-2-ylmethyl-4-1H-pyrazolyl, 2,4-dioxo-1,2,34-tetrahydro-5-pyrimidinyl, 2,4-di(tert-butoxy)pyrimidin-5-yl, 2-ethoxypyridin-3-yl, 2-fluoro-3-methylpyridin-5-yl, 2-fluoropyridin-3-yl, 2-fluoropyridin-5-yl, 5-formyl-2-furyl, 5-formylthiophen-2-yl, furan-3-yl, furan-2-yl, 5-indolyl, isoquinolin-4-yl, 2-methoxypyrimidin-5-yl, 5-methyl-1-benzothiophen-2-yl, 5-methylfuran-2-yl, 5-methyl-3-phenyl-4-isoxazolyl, 5-(methylsulfanyl)-2-thienyl, 3-methyl-pyridin-2-yl, (5-methyl)thien-2-yl, 5-methylpyridin-2-yl, 5-methylpyridin-3-yl, 2-methoxypyridine-3-yl, (4-methyl)thien-2-yl, 2-methoxypyridin-5-yl, 1-(phenylsulfonyl)-1H-indol-3-yl, 1-(phenylsulfonyl)-1H-indol-3-yl, 5-phenyl-2-thienyl, pyridin-4-yl, pyridin-3-yl, 5-pyrimidinyl, 4-phenoxathiinyl, 8-quinolinyl, 3-quinolinyl, 1-tert-butoxycarbonyl-2-pyrrolyl, 1-(tert-butoxycarbonyl)-5-bromo-1H-indol-2-yl, 1-(tert-butoxycarbonyl)-1H-indol-2-yl, 1-(tert-butoxycarbonyl)-5-methoxy-1H-indol-2-yl, 1-thianthrenyl-3-thienyl, or 2-thienyl.

FIG. 2 schematically shows a further alternative method for making 2,4,6-trisubstituted pteridines. In step (a), treatment of 5,6-diamino-2-mercapto-pyrimidin-4-ol with a substituted α-keto aldoxime in the presence of a strong acid in an alcoholic solvent provides the 2-mercapto-4-hydroxy-6-substituted pteridine. Reaction with appropriately substituted α-keto aldoximes allows the introduction on position 6 of the pteridinie core structure of a variety of substitutents such as optionally substituted aryl or heteroaryl groups, in particular for substituents such as those described for compounds represented by structural formulae VII to X, XII and XIV.

In step (b), the sulfhydryl group at position 2 is alkylated with an alkylating reagent such as methyl iodide in the presence of a base, such as a metal alkoxide, in an aprotic solvent. In step (c), the desired 4-substituent is introduced. The 2-alkylthio-4-hydroxypteridine (resulting from step (b)) is activated on position 4 by introduction of a leaving group, e.g. with a typical coupling reagent such as benzotriazol-1-yloxy-tris(dimethylamino)phosphonium hexafluorophosphate in a dipolar aprotic solvent in the presence of an organic base such as a tertiary amine. Subsequently the activated compound is treated with the requisite nucleophilic reagent such as known to the person skilled in the art, e.g. an amine or alcohol. Finally, in step (d), the desired 2-substituent is introduced by (nucleophilic) displacement of the 2-alkylthio moiety with the requisite amine in a dipolar aprotic solvent in the presence of an organic base such as a tertiary amine, facilitated by microwave irradiation. In particular, for the purpose of the present invention, the requisite amine corresponds to the formulae H₂N—CHR₆R₇ or H₂N—R₈, wherein R₆, R₇ and R₈ are as defined for structural formulae VI, VIII, X, XI, XIII and XIV. Alternatively the 2-alkylthio moiety may be oxidized to the sulfoxide or sulfone by treatment with an oxidant such as an organic peracid prior to displacement by the requisite amine. Suitable commercially available reagents for use in step (d) include, but are not limited to, 2-chlorobenzylamine, 4-chlorobenzylamine, 2,4-dichlorobenzylamine, 3,4-dichlorobenzylamine, 4-methoxybenzylamine, 4-methylbenzylamine, piperonylamine, 3,4-dimethoxybenzylamine, 3-methylbenzylamine, 3-fluorobenzylamine, 2-methylbenzylamine, 2-methoxybenzylamine, 3-methoxybenzylamine, 2-fluorobenzylamine, 4-fluorobenzylamine, 3,4-dihydroxybenzylamine, 3-chlorobenzylamine, 4-(trifluoromethoxy)benzylamine, 2,6-difluorobenzylamine, 3,5-bis(trifluoromethyl)benzylamine, 2,4-difluorobenzylamine, 2,5-difluorobenzylamine, 3,4-difluorobenzylamine, 2-(trifluoromethyl)benzylamine, 3-(trifluoromethyl)benzylamine, 2-bromobenzylamine, 4-bromobenzylamine, 2-chloro-6-fluorobenzylamine, 2,5-dimethylbenzylamine, 3,4,5-Trimethoxybenzylamine, 2,4,6-Trimethylbenzylamine, 2,4-Dimethylbenzylamine, 2,3-Dichlorobenzylamine, 1-Naphthalenemethylamine, 3-Iodobenzylamine, 2-Hydroxybenzylamine, 3-Bromobenzylamine, 2,6-Dichlorobenzylamine, 3,4-Dihydro-2H-1,5-benzodioxepin-6-ylmethylamine, 2,3-Dihydro-1,4-benzodioxin-6-ylmethylamine, 2,3-Dihydro-1,4-benzodioxin-5-ylmethylamine, 1-Benzofuran-5-ylmethylamine, 4-(2-Thienyl)benzylamine, 3,4-Dihydro-2H-1,5-benzodioxepin-7-ylmethylamine, 4-Morpholinobenzylamine, 4-(1H-Pyrazol-1-yl)benzylamine, 4-(4-Methylpiperazino)benzylamine, 2-Piperidinobenzylamine, 3-(1H-Pyrrol-1-yl)benzylamine, 2-Morpholinobenzylamine, 4-(1H-Pyrrol-1-yl)benzylamine, 2-Chloro-6-phenoxybenzylamine, 2-(Methylthio)benzylamine, 2-(Trifluoromethoxy)benzylamine, 2,3-Dimethylbenzylamine, 4-(Trifluoromethyl)benzylamine, 3,5-Dichlorobenzylamine, 2-(Aminomethyl)-3-fluoroaniline, 3-Chloro-4-fluorobenzylamine, 2,5-Dimethoxybenzylamine, 2,5-Dichlorobenzylamine, 2,6-Dimethoxybenzylamine, 2,4-Dichloro-6-methylbenzylamine, 3-Chloro-4-methylbenzylamine, 4-Fluoro-3-(trifluoromethyl)benzylamine, 4-Fluoro-2-(trifluoromethyl)benzylamine, 3-Piperidin-1-ylmethyl benzylamine, 1-Benzothiophen-5-ylmethylamine, 4-(Morpholinomethyl)benzylamine, (3-((4-Methylpiperidino)methyl)phenyl)methanamine, (4-Piperidinophenyl)methylamine, (3-Piperidinophenyl)methylamine, 1-[2-(4-Methylpiperazin-1-yl)phenyl]methanamine, (1,4-Dimethyl-1,2,3,4-tetrahydroquinoxalin-6-yl)methylamine, 3-(Trifluoromethoxy)benzylamine, 4-bromo-2-fluorobenzylamine, 2-(1 h-pyrazol-1-yl)benzylamine, tert-butyl 4-(2-(aminomethyl)phenyl)piperazine-1-carboxylate, (3-Morpholinophenyl)methylamine, tert-Butyl N-[4-(aminomethyl)phenyl]carbamate, [2-(1H-Pyrrol-1-yl)phenyl]methylamine, 1-[3-(4-Methylpiperazin-1-yl)phenyl]methanamine, [4-(1-Pyrrolidinyl)phenyl]methanamine, (3-pyrrolidin-1-ylphenyl)methylamine, [4-(2-morpholinoethoxy)phenyl]methylamine, [2-(2-Morpholinoethoxy)phenyl]methylamine, [3-(2-Morpholinoethoxy)phenyl]methylamine, [3-(morpholinomethyl)phenyl]methylamine, [4-(piperidinomethyl)phenyl]methylamine, {4-[(4-Methylpiperazin-1-yl)methyl]phenyl}methylamine, [4-(2-furyl)phenyl]methylamine, tert-butyl 4-[4-(aminomethyl)phenyl]tetrahydro-1(2H)-pyrazinecarboxylate, (2,2-dimethyl-2,3-dihydro-1-benzofuran-7-yl)methylamine, [3-(1H-1,2,4-triazol-1-yl)phenyl]methylamine, (4-thien-3-ylphenyl)methylamine, 1-[2-(morpholin-4-ylmethyl)phenyl]methanamine, {2-[(4-methylpiperazin-1-yl)methyl]phenyl}methylamine, [3-(2-furyl)phenyl]methylamine, (3-thien-2-ylphenyl)methylamine, [2-(2-furyl)phenyl]methylamine, 4-(Pyrrolidin-1-ylmethyl)benzylamine, 4-[(4-methylperhydro-1,4-diazepin-1-yl)methyl]benzylamine, 4-[2-(dimethylamino)ethoxy]benzylamine, (2-Pyrrolidin-1-ylphenyl)methylamine, [3-(1-Pyrrolidinylmethyl)phenyl]methanamine, (3-thien-3-ylphenyl)methylamine, 2-[2-(Dimethylamino)ethoxy]benzylamine, 2-(phenoxymethyl)benzylamine, (1-methyl-1h-indol-4-yl)methylamine, 4-(4-methylperhydro-1,4-diazepin-1-yl)benzylamine, (1-Methyl-1H-indol-6-yl)methylamine, [3-(1,3-thiazol-2-yl)phenyl]methylamine, 3-(1H-Pyrazol-1-ylmethyl)benzylamine, (1-Methyl-1H-indol-5-yl)methylamine, 3-(Phenoxymethyl)benzylamine, 2-Morpholino-5-(trifluoromethyl)benzylamine, [4-(1,3-Thiazol-2-yl)phenyl]methylamine, 3-(1-Methyl-1H-pyrazol-3-yl)benzylamine, 2-(4-Methylperhydro-1,4-diazepin-1-yl)benzylamine, 4-[3-(Dimethylamino)propoxy]benzylamine, 3-(2-Methyl-1H-imidazol-1-yl)benzylamine, 4-(2-Methyl-1H-imidazol-1-yl)benzylamine, 2-(2-Methyl-1H-imidazol-1-yl)benzylamine, [4-(Tetrahydropyran-4-yloxy)phenyl]methylamine, 3-[3-(Dimethylamino)propoxy]benzylamine, 2-[3-(Dimethylamino)propoxy]benzylamine, 3-pyrimidin-2-ylbenzylamine, 4-(1-Methyl-1H-pyrazol-3-yl)benzylamine and 3-(1-methyl-1h-pyrazol-5-yl)benzylamine and 1-(1-benzothien-7-yl)methanamine.

The present invention will be further described with reference to certain more specific embodiments and examples, but the present invention is not limited thereto. The following examples are given by way of illustration only. HPLC retention times are reported using the following chromatographic method:

| | |
|---|---|
| Column: | Phenomenex Gemini 5μ C18 |
| Dimensions: | 30 × 4.6 mm |
| Mobile Phase: | A) Water with 0.1% Formic Acid |
| | B) Acetonitrile with 0.1% Formic Acid |
| Gradient: | A/B (95:5) to B (100%) in 3 min |
| Flow Rate: | 2 mL/min |
| Temperature: | 30° C. |
| Detection: | UV @ 254 nm |

### Example A

### Synthesis of 2-amino-4-(N-carbamoyl-piperazin-1-yl)-6-(3,4-dimethoxy-phenyl)pteridines

To a solution of 2-amino-4-(N-piperazin-1-yl)-6-(3,4-dimethoxyphenyl)-pteridine (214 mg, 0.583 mmole) in dimethylformamide (30 ml) was added a suitable isocyanate (0.76 mmole). The solution was stirred at room temperature for 16 hours. The solvent was then evaporated in vacuo and the crude residue was purified by silica gel flash chromatography, the mobile phase being a mixture of methanol and dichloromethane (in a volume ratio gradually ranging from 2:98 to 5:95), resulting in the pure title compounds, each being characterized by its mass spectrum (MS), in yields varying from 65 to 85%, depending upon the isocyanate used. The following compounds were synthesized according to this procedure:
2-amino-4-(N-3-chloro-phenyl-carbamoyl-piperazin-1-yl)-6-(3,4-dimethoxyphenyl)pteridine (example 122, Table 1) was obtained from 3-chloro-phenyl isocyanate; MS (m/z): 521, 523 ([M+H]⁺, 100);
2-amino-4-(N-3-trifluoromethyl-phenyl-carbamoyl-piperazin-1-yl)-6-(3,4-dimethoxyphenyl)pteridine (example 123, Table 1) was obtained from 3-trifluoromethyl phenyl isocyanate; MS (m/z): 555 ([M+H]⁺, 100);
2-amino-4-(N-4-bromo-phenyl-carbamoyl-piperazin-1-yl)-6-(3,4-dimethoxyphenyl)pteridine (example 124, Table 1) was obtained from 4-bromo-phenyl isocyanate; MS (m/z): 565, 567 ([M+H]⁺, 100)

### Example B

### Synthesis of 2-amino-4-[4-(4-methylphenyl)piperazinyl]-6-(4-isopropoxy-3-methoxy-phenyl)pteridine (Example 125, Table 1)

A purple suspension of 2,4-diamino-6-hydroxy-5-nitrosopyrimidine (5.05 g, 31.6 mmole) in water (80 ml) and NH₄OH (6.4 ml of a 30% aqueous solution) was stirred at room temperature for 20 minutes. Then, sodium dithionite (16.6 g, 82 mmole, technical grade 86%) was added under vigorous stirring and the reaction mixture was stirred at 80° C. for 16 hours. The mixture was filtered while still hot, the filtrate was allowed to cool down to room temperature and then placed at 4° C. overnight. The precipitate formed was filtered off, washed respectively with cold water, methanol and diethyl ether, and dried to provide 2,4,5-triamino-6-hydroxy-pyrimidine (3.72 g, yield 83%) which was used as such for the following reactions.

To a suspension of 2,4,5-triamino-6-hydroxy-pyrimidine (17.2 mmole) and 4-acetoxy-3-methoxyphenylglyoxalmonoxime (2.43 g, 17.2 mmole) in methanol (400 ml) was added a 1.25 M solution of HCl in methanol (28 ml, 35.0 mmole). The mixture was heated at reflux and the reaction was monitored by thin layer chromatography (TLC) for disappearance of both starting materials. After 5 days, another aliquot of the HCl solution was added. After an additional 5 days, the reaction mixture was allowed to cool down to room temperature. The precipitate was filtered off, washed with methanol and dried, thus providing 2-amino-4-hydroxy-6-(4-isopropoxy-3-methoxy-phenyl)pteridine (2.01 g, yield 41%), which was used as such for the next reaction and was characterized by its mass spectrum as follows: MS (m/z): 286 ([M+H]⁺, 100).

A suspension of 2-amino-4-hydroxy-6-(4-isopropoxy-3-methoxy-phenyl) pteridine (580 mg, 1.8 mmole), 1-(4-methylphenyl)piperazine (1.64 g, 9.2 mmole), p-toluenesulfonic acid monohydrate (41 mg, 0.21 mmole), ammonium sulfate (50 mg, 0.38 mmole) and 1,1,1,3,3,3-hexamethyldisilazane (1.94 ml, 9.0 mmole) in toluene (30 ml) was heated at reflux for 2 days. Upon cooling, the reaction mixture was evaporated with silica gel and purified twice on a silica gel column (10% methanol in dichloromethane with 1% triethylamine) to afford the pure title compound (445 mg, yield 51%) which was characterized by its mass spectrum as follows: MS (m/z): 486 ([M+H]⁺, 100).

### Example C

### Synthesis of 4-ethoxy-6-(4-fluorophenyl)-pteridin-2-yl]-(4-fluorobenzyl)-amine

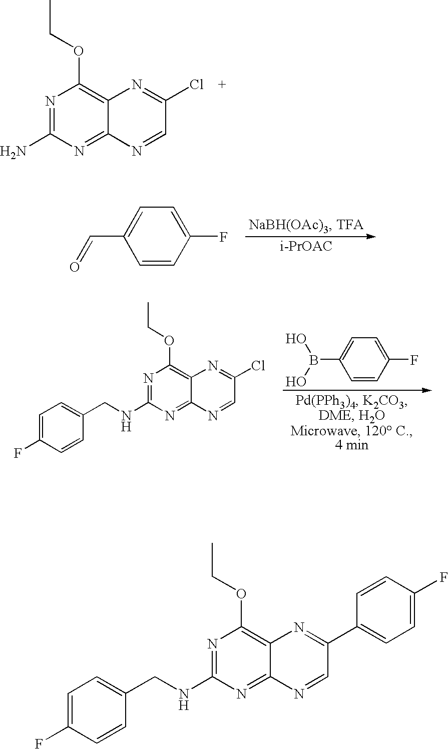

In a first step, a mixture of 6-chloro-4-ethoxy-pteridin-2-ylamine (50 mg, 0.22 mmol), 4-fluorobenzylaldehyde (85 μL, 0.88 mmol), trifluoroacetic acid (0.17 mL, 2.2 mmol) and NaBH(OAc)₃ (140 mg, 0.66 mmol) in isopropyl acetate (1.5 mL) was heated to 120° C. for 30 minutes. After cooling, the reaction mixture was quenched by the addition of saturated aqueous NaHCO₃. The solution was partitioned with ethyl acetate. The organic layer was dried over Na₂SO₄ and concentrated to afford the crude product which was purified by RP HPLC using a C18 column with a gradient of H₂O, 0.05% TFA-acetonitrile, to provide (6-chloro-4-ethoxy-pteridin-2-yl)-(4-fluoro-benzyl)-amine as white solid (17 mg, yield: 23%) which was characterised as follows:

MS (m/z) 226.0 [M+H]⁺; and

HPLC Rₜ=1.72 min.

In a second step, a mixture of (6-chloro-4-ethoxy-pteridin-2-yl)-(4-fluoro-benzyl)-amine (17 mg, 0.05 mmol), potassium carbonate (28 mg, 0.2 mmol), tetrakis(triphenylphosphine) palladium (6 mg) and p-fluorophenylboronic acid (7 mg, 0.05 mmol) in DME (1 mL) and water (0.5 mL) was heated to 120° C. for 4 minutes by microwave. Solvents were concentrated in vacuo and the residue was purified by RP HPLC using a C18 column with a gradient of H₂O, 0.05% TFA-acetonitrile, to provide 3.2 mg of [4-Ethoxy-6-(4-fluoro-phenyl)-pteridin-2-yl]-(4-fluoro-benzyl)-amine which was characterised as follows:

MS (m/z) 394.2 [M+H]⁺;

HPLC Rₜ=2.74 min.; and

¹H-NMR (300 MHz, DMSO-d₆) δ 1.41 (3H, t), 4.55 (4H, m), 7.09 (2H, dd), 7.33 (4H, m), 8.18 (2H, dd) and 9.36 (1H, s) ppm.

### Example D

### Synthesis of 4-{[6-(4-fluoro-phenyl)-4-(2,2,2-trifluoro-ethylamino)-pteridin-2-ylamino]-methyl}-benzenesulfonamide

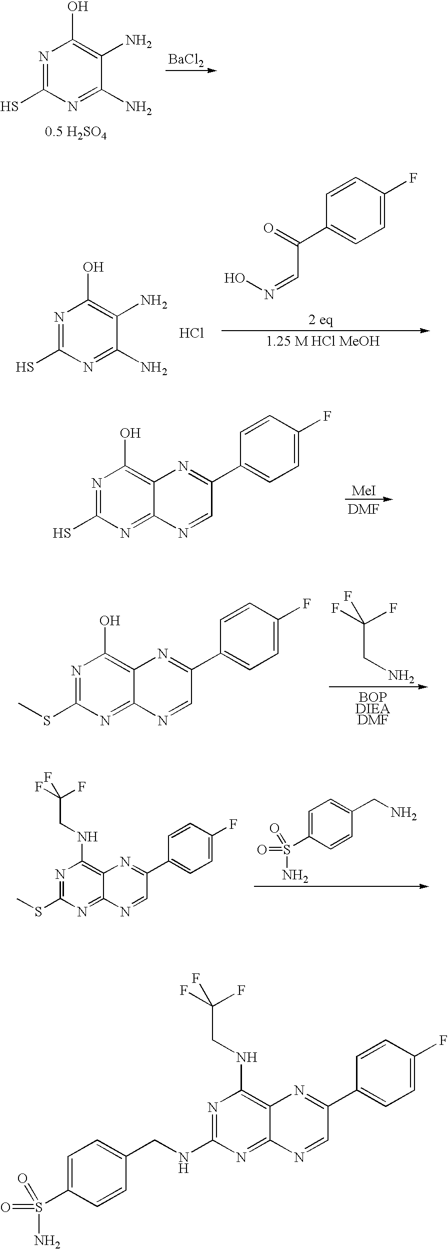

To a suspension of 4,5-Diamino-6-hydroxy-2-mercaptopyrimidine hemisulfate hydrate (15 g, 72 mmol) in water (200 mL) at 80° C. was added dropwise a solution of barium chloride dihydrate (8.8 g, 36 mmol) in water (100 mL). The resulting suspension was stirred for 30 minutes at 80° C. The reaction mixture was cooled to room temperature and barium sulfate was removed by filtration over diatomaceous earth. The filtrate was frozen and lyophilized to provide 9.22 g (66% yield) of 5,6-diamino-2-mercapto-pyrimidin-4-ol hydrochloride as beige solid.

To a boiling solution of 5,6-diamino-2-mercapto-pyrimidin-4-ol hydrochloride (1.22 g, 6.3 mmol) in methanol (60 mL) was added dropwise a solution of 4-fluorophenylglyoxal mono-oxime (1.08 g, 6.5 mmol) in methanol (10 mL). The reaction mixture was heated under reflux for 4 hours. The resulting precipitate was filtered, washed with water, ethanol and diethyl ether, and dried under vacuum, providing 6-(4-fluoro-phenyl)-2-mercapto-pteridin-4-ol as a yellow solid (0.68 g, yield: 56%) which was characterised as follows:

MS (m/z) 275.0 [M+H]⁺;

HPLC Rₜ=1.98 min; and

¹H-NMR (300 MHz, DMSO-d₆) δ 7.35 (2H, dd), 8.17 (2H, dd) and 9.28 (1H, s) ppm.

A mixture of 6-(4-fluoro-phenyl)-2-mercapto-pteridin-4-ol (337 mg, 1.23 mmol) and iodomethane (150 μL, 2.46 mmol) in DMF (5 mL) was stirred at room temperature overnight. Water (15 mL) was added to the reaction mixture, the precipitate formed was filtered and washed with diethyl ether to give 6-(4-fluoro-phenyl)-2-methylsulfanyl-pteridin-4-ol as a beige solid (281 mg, yield: 80%) which was characterised as follows:

MS (m/z) 289.1 [M+H]⁺;

HPLC Rₜ=2.01 min.; and

¹H-NMR (300 MHz, DMSO-d₆) δ 2.57 (3H, s), 7.36 (2H, dd), 8.24 (2H, dd) and 9.46 (1H, s) ppm.

A mixture of 6-(4-fluoro-phenyl)-2-methylsulfanyl-pteridin-4-ol (281 mg, 0.98 mmol), trifluoroethylamine (230 μL, 2.92 mmol), BOP (520 mg, 1.18 mmol) and DIEA (200 μL, 1.18 mmol) in DMF (5 mL) was stirred at room temperature overnight. Water (25 mL) was added to the reaction mixture. The precipitate so formed was filtered and washed with diethyl ether and dichloromethane to give [6-(4-fluoro-phenyl)-2-methylsulfanyl-pteridin-4-yl]-(2,2,2-trifluoro-ethyl)-amine as a yellow solid (260 mg, yield: 99%) which was characterised as follows:

MS (m/z) 369.9 [M+H]⁺; and

HPLC Rₜ=2.69 min.

A mixture of [6-(4-fluoro-phenyl)-2-methylsulfanyl-pteridin-4-yl]-(2,2,2-trifluoro-ethyl)-amine (55 mg, 0.15 mmol), 4-aminomethyl-benzenesulfonamide hydrochloride (331 mg, 1.5 mmol), and DIEA (0.5 mL, 3 mmol) in NMP (1.5 mL) was heated to 220° C. for 1.5 hour using microwave irradiation. Water was added and the resulting yellow precipitate was collected by vacuum filtration. The crude solid was purified by RP HPLC using a C18 column with a gradient of H₂O, 0.05% TFA-acetonitrile, to provide 26 mg of 4-{[6-(4-fluoro-phenyl)-4-(2,2,2-trifluoro-ethylamino)-pteridin-2-ylamino]-methyl}-benzenesulfonamide which was characterised as follows:

MS (m/z) 508.2 [M+H]⁺;

HPLC Rₜ=1.99 min.; and

¹H-NMR (300 MHz, CD₃OD) δ 4.36 (4H, m), 7.26 (2H, m), 7.55 (2H, d), 7.86 (2H, d), 8.31 (2H, m) and 9.37 (1H, s) ppm.

### Example E

### Synthesis of N2-(4-Fluoro-benzyl)-6-(4-fluoro-phenyl)-N4-(2,2,2-trifluoro-ethyl)-pteridine-2,4-diamine

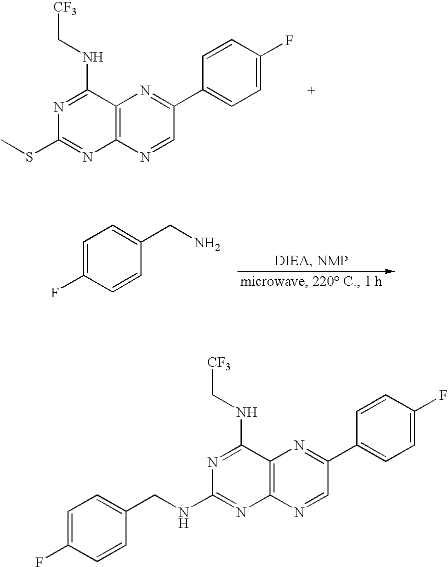

The procedure of 4-{[6-(4-fluoro-phenyl)-4-(2,2,2-trifluoro-ethylamino)-pteridin-2-ylamino]-methyl}-benzenesulfonamide was repeated, except for the use of 4-fluorobenzylamine instead of 4-aminomethyl-benzenesulfonamide hydrochloride. The title compound was characterised as follows:

MS (m/z) 447.2 [M+H]⁺;

HPLC Rₜ=2.34 min.; and

¹H-NMR (300 MHz, CD₃OD) δ 4.40 (2H, m), 4.75 (2H, s), 7.05 (2H, dd), 7.26 (2H, dd), 7.40 (2H, dd), 8.30 (2H, m) and 9.33 (1H, s) ppm.

### Example F

### Synthesis of 4-{1-[6-(4-Fluoro-phenyl)-4-(2,2,2-trifluoro-ethylamino)-pteridin-2-ylamino]-ethyl}-benzenesulfonamide

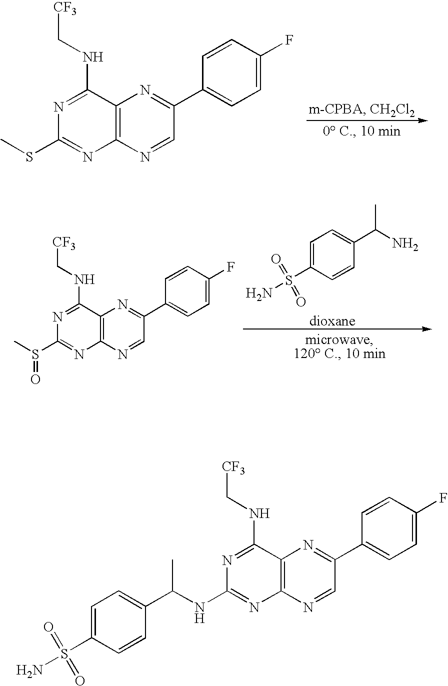

To a mixture of [6-(4-Fluoro-phenyl)-2-methylsulfanyl-pteridin-4-yl]-(2,2,2-trifluoro-ethyl)-amine (40 mg, 0.11 mmol) in dichloromethane (2 mL) at 0° C. was added m-chloroperbenzoic acid (38 mg, 0.22 mmol). The reaction was kept at 0° C. for 10 minutes. The reaction was quenched by adding 10% aqueous NaS₂O₃ solution, then partitioned between saturated aqueous sodium bicarbonate solution and ethyl acetate. The combined organic layers were dried over sodium sulfate and concentrated to provide 42 mg of [6-(4-fluoro-phenyl)-2-methanesulfinyl-pteridin-4-yl]-(2,2,2-trifluoro-ethyl)-amine, which was carried to the next step without further purification and was characterised as follows:

MS (m/z) 408.1 [M+H]⁺; and

HPLC Rₜ=2.15 min.

A mixture of [6-(4-fluoro-phenyl)-2-methanesulfinyl-pteridin-4-yl]-(2,2,2-trifluoro-ethyl)-amine (42 mg, 0.11 mmol) and 4-(1-amino-ethyl)-benzenesulfonamide (33 mg, 0.16 mmol) in dioxane (1 mL) was heated to 120° C. for 10 minutes using microwave irradiation. The mixture was partitioned between saturated aqueous sodium bicarbonate solution and ethyl acetate. The combined organic layers were washed with brine, dried over sodium sulfate and concentrated. The residue was purified by RP HPLC using a C18 column with a gradient of H₂O, 0.05% TFA-acetonitrile, to provide 14.5 mg (25% yield) of 4-{1-[6-(4-Fluoro-phenyl)-4-(2,2,2-trifluoro-ethylamino)-pteridin-2-ylamino]-ethyl}-benzenesulfonamide which was characterised as follows:

MS (m/z) 522.2 [M+H]⁺;

HPLC Rₜ=2.15 min; and

¹H-NMR (300 MHz, CD₃OD) δ 1.63 (3H, d), 4.39 (2H, m), 5.39 (1H, m), 7.25 (2H, dd), 7.58 (2H, dd), 7.86 (2H, dd), 8.30 (2H, m) and 9.36 (1H, s) ppm.

### Example G

### Synthesis of 4-{[4-Ethoxy-6-(4-fluoro-phenyl)-pteridin-2-ylamino]-methyl}-benzenesulfonamide

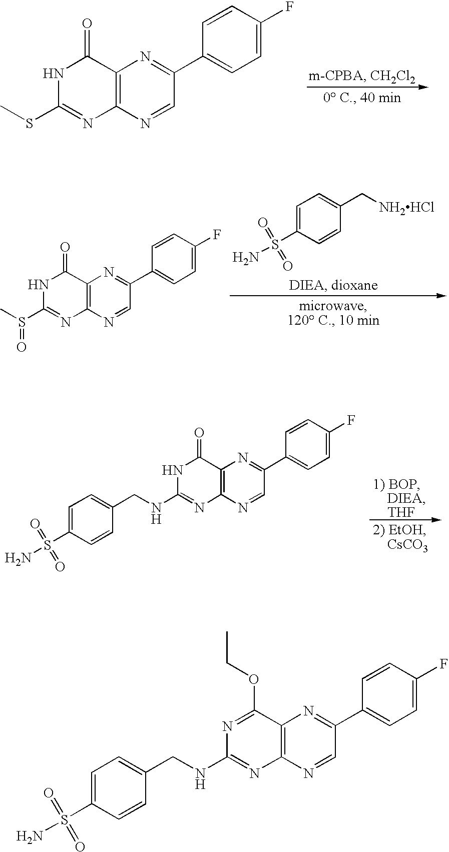

### 6-(4-fluoro-phenyl)-2-methanesulfinyl-3H-pteridin-4-one

The procedure of [6-(4-fluoro-phenyl)-2-methanesulfinyl-pteridin-4-yl]-(2,2,2-trifluoro-ethyl)-amine was repeated, except for the use of 6-(4-fluoro-phenyl)-2-methylsulfanyl-3H-pteridin-4-one instead of [6-(4-fluoro-phenyl)-2-methylsulfanyl-pteridin-4-yl]-(2,2,2-trifluoro-ethyl)-amine. The title compound was characterised as follows: MS (m/z) 305.0 [M+H]⁺; HPLC Rₜ=1.79 min.

### 4-{[6-(4-Fluoro-phenyl)-4-oxo-3,4-dihydro-Pteridin-2-ylamino]-methyl}-benzenesulfonamide

A mixture of 6-(4-Fluoro-phenyl)-2-methylsulfanyl-3H-pteridin-4-one (213 mg, 0.7 mmol), 4-aminomethyl-benzenesulfonamide hydrochloride (230 mg, 1.05 mmol) and DIEA (360 μL, 2.1 mmol) in dioxane (6 mL) was heated to 120° C. for 10 minutes using microwave irradiation. The mixture was partitioned between saturated aqueous sodium bicarbonate solution and ethyl acetate. The combined organic layers were washed with brine, dried over sodium sulfate and concentrated to dryness. The crude product was carried to the next step without further purification but was characterised as follows: MS (m/z) 427.1 [M+H]⁺; HPLC Rₜ=1.94 min.

### 4-{[4-Ethoxy-6-(4-fluoro-phenyl)-pteridin-2-ylamino]-methyl}-benzenesulfonamide

4-{[6-(4-Fluoro-phenyl)-4-oxo-3,4-dihydro-pteridin-2-ylamino]-methyl}-benzenesulfonamide (256 mg, 0.6 mmol) and BOP (531 mg, 1.2 mmol) were mixed in THF (10 mL), and DIEA (314 μL, 1.8 mmol) was added. The reaction mixture was stirred at room temperature for 2 hours. This solution was then transferred to a stirred solution of sodium ethoxide (1 mL, 3 mmol, 21 wt % in ethanol) in ethanol (10 mL). After stirring at room temperature for 30 min, the THF was removed in vacuo and the residue diluted with ethyl acetate (100 mL) and washed with water and brine. The organic layer was dried over sodium sulfate and concentrated to afford an oily residue, which was purified by RP HPLC using a C18 column with a gradient of H₂O, 0.05% TFA-acetonitrile, to provide 4.5 mg of the desired product which was characterised as follows: MS (m/z) 455.2 [M+H]⁺; HPLC Rₜ=2.33 min.

### Example H

### Synthesis of N-Cyclopropyl-4-[2-(4-sulfamoyl-benzylamino)-4-(2,2,2-trifluoro-ethylamino)-pteridin-6-yl]-benzamide

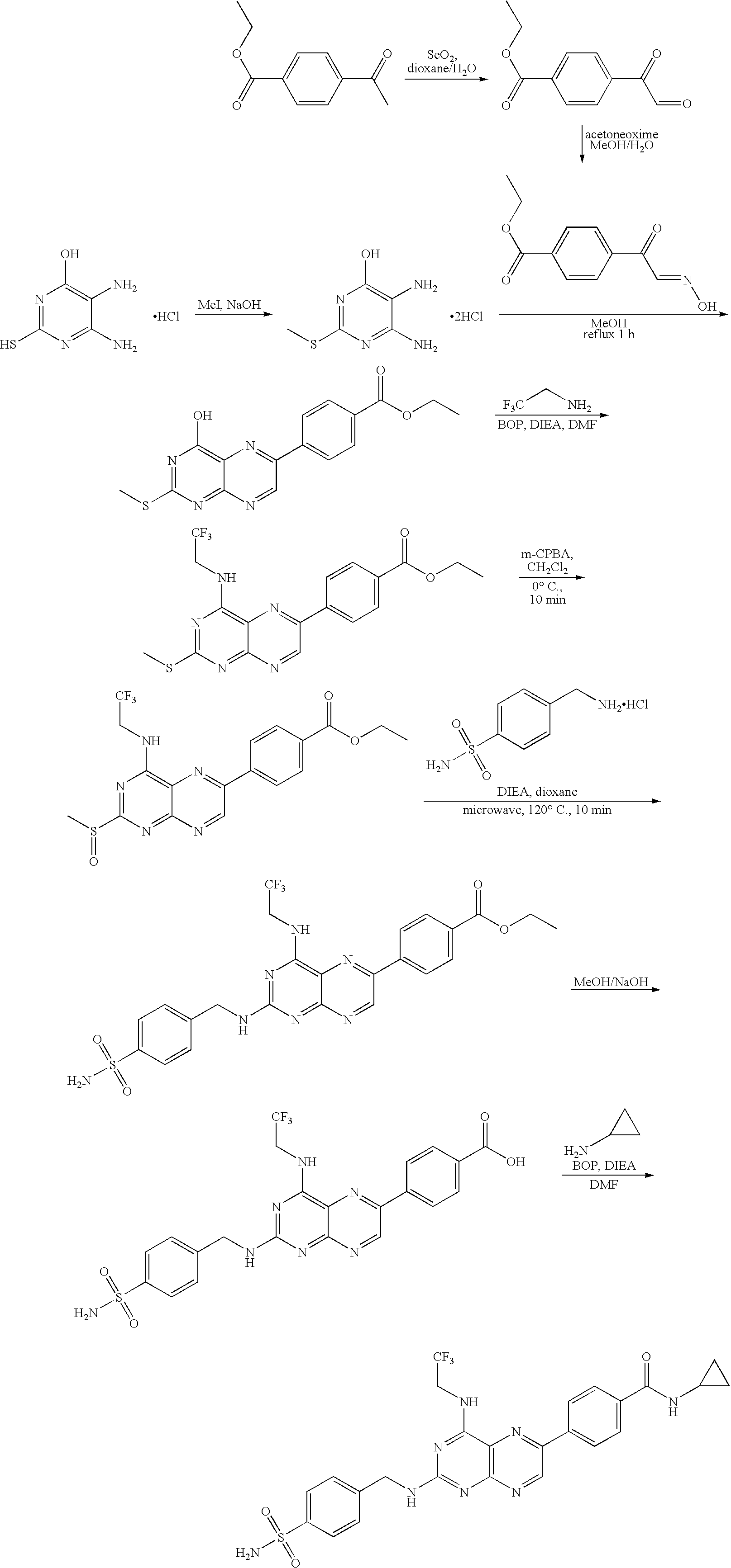

### 5,6-Diamino-2-methylsulfanyl-pyrimidin-4-ol dihydrochloride

Iodomethane (5.4 mL, 87.34 mmol) was added to a solution of 5,6-Diamino-2-mercapto-pyrimidin-4-ol hydrochloride (8.5 g, 13.67 mmol) in 1N NaOH (200 mL). After 30 min, the solution was acidified by addition of 6N HCl and concentrated to dryness. The crude yellow solid was triturated with EtOH. Insoluble material was removed by filtration and the filtrate was concentrated to provide 10 g of the desired product as a yellow solid which was characterised as follows: MS (m/z) 173.2 [M+H]⁺; HPLC Rₜ=0.34 min.

### 4-(2-Hydroxyimino-acetyl)-benzoic acid ethyl ester

SeO₂ (2.86 g, 25.7 mmol) and 4-Acetyl-benzoic acid ethyl ester (4.55 g, 23.4 mmol) were suspended in a mixture of dioxane (200 mL) and water (8 mL) and the solution was heated under reflux for 24 hours. The hot solution was filtered and the filtrate was evaporated to dryness. The oily residue was purified by silica gel chromatography eluting with hexane/ethyl acetate to give 4-(2-oxo-acetyl)-benzoic acid ethyl ester. This material was suspended in a mixture of water (200 mL) and MeOH (50 mL). Then acetonoxime (1.68 g, 23 mmol) was added and the mixture was heated to 50° C. for 2 hours. The precipitate was collected by vacuum filtration to afford 3.1 μg (yield 60% for 2 steps) of the product as a white solid which was characterised as follows:

MS (m/z) 222.2 [M+H]⁺;

HPLC Rₜ=2.26 min.; and

¹H-NMR (300 MHz, DMSO-d₆) δ 1.28 (3H, t), 4.29 (2H, q) and 8.01 (4H, m) ppm.

### 4-(4-Hydroxy-2-methylsulfanyl-pteridin-6-yl)-benzoic acid ethyl ester

To a boiling solution of 5,6-Diamino-2-methylsulfanyl-pyrimidin-4-ol dihydrochloride (1.22 g, 5 mmol) in methanol (60 mL) was added dropwise a solution of 4-(2-Hydroxyimino-acetyl)-benzoic acid ethyl ester (1.1 g, 5 mmol) in methanol (10 mL). The reaction mixture was heated under reflux for 1 hour. The precipitate formed was filtered, washed with water, ethanol and diethyl ether and dried under vacuum, providing the title compound as a white solid (1 g, yield: 58%) which was characterised as follows:

MS (m/z) 341.2 [M−H]⁻;

HPLC Rₜ=2.21 min.; and

¹H-NMR (300 MHz, DMSO-d₆) δ 1.30 (3H, t), 2.58 (3H, s), 4.30 (2H, q), 8.06 (2H, d), 8.32 (2H, d) and 9.52 (1H, s) ppm.

### 4-[2-Methylsulfanyl-4-(2,2,2-trifluoro-ethylamino)-pteridin-6-yl]-benzoic acid ethyl ester

A mixture of 4-(4-Hydroxy-2-methylsulfanyl-pteridin-6-yl)-benzoic acid ethyl ester (0.65 g, 1.9 mmol), trifluoroethylamine (0.5 mL, 5.7 mmol), BOP (1.26 g, 2.85 mmol) and DIEA (1.8 mL, 10.45 mmol) in DMF (10 mL) was stirred at room temperature overnight. Water (25 mL) was added to the reaction mixture and the precipitate so formed was filtered and washed with 10% ACN/water to give the title compound as a yellow solid (760 mg, yield: 88%) which was characterised as follows: MS (m/z) 424.1 [M+H]⁺; HPLC Rₜ=2.81 min.

### 4-[2-Methanesulfinyl-4-(2,2,2-trifluoro-ethylamino)-pteridin-6-yl]-benzoic acid ethyl ester

The procedure for the synthesis of [6-(4-Fluoro-phenyl)-2-methanesulfinyl-pteridin-4-yl]-(2,2,2-trifluoro-ethyl)-amine was repeated, except for the use of 4-[2-Methylsulfanyl-4-(2,2,2-trifluoro-ethylamino)-pteridin-6-yl]-benzoic acid ethyl ester instead of [6-(4-Fluoro-phenyl)-2-methylsulfanyl-pteridin-4-yl]-(2,2,2-trifluoro-ethyl)-amine. The title compound was characterised as follows: MS (m/z) 440.2 [M+H]⁺; HPLC Rₜ=2.31 min.

### 4-[2-(4-Sulfamoyl-benzylamino)-4-(2,2,2-trifluoro-ethylamino)-pteridin-6-yl]-benzoic acid ethyl ester

The procedure for the synthesis of 4-{[6-(4-Fluoro-phenyl)-4-oxo-3,4-dihydro-pteridin-2-ylamino]-methyl}-benzenesulfonamide was repeated, except for the use of 4-[2-Methanesulfinyl-4-(2,2,2-trifluoro-ethylamino)-pteridin-6-yl]-benzoic acid ethyl ester instead of 6-(4-Fluoro-phenyl)-2-methylsulfinyl-3H-pteridin-4-one. The title compound was characterised as follows:

MS (m/z) 562.1 [M+H]⁺;

HPLC Rₜ=2.23 min.; and

¹H-NMR (300 MHz, DMSO-d₆) δ 1.26 (3H, t), 4.26 (2H, q), 4.37 (2H, q), 4.78 (2H, s), 7.50 (2H, d), 7.75 (2H, d), 8.06 (2H, d), 8.46 (2H, d) and 9.55 (1H, s) ppm.

### 4-[2-(4-Sulfamoyl-benzylamino)-4-(2,2,2-trifluoro-ethylamino)-pteridin-6-yl]-benzoic acid

A suspension of 4-[2-(4-Sulfamoyl-benzylamino)-4-(2,2,2-trifluoro-ethylamino)-pteridin-6-yl]-benzoic acid ethyl ester (225 mg, 0.4 mmol) in MeOH (10 mL) and 2N NaOH (10 mL) was stirred at room temperature for 30 minutes. The mixture was acidified with 6N HCl and then partitioned between ethyl acetate and water. The organic phase was dried over sodium sulfate, filtered and concentrated under reduced pressure to yield 71 mg of crude product which was not purified but used as such for further reactions and was characterised as follows: MS (m/z) 534.1 [M+H]⁺; HPLC Rₜ=1.90 min.

### N-Cyclopropyl-4-[2-(4-sulfamoyl-benzylamino)-4-(2,2,2-trifluoro-ethylamino)-pteridin-6-yl]-benzamide

A mixture of 4-[2-(4-Sulfamoyl-benzylamino)-4-(2,2,2-trifluoro-ethylamino)-pteridin-6-yl]-benzoic acid (69 mg g, 0.13 mmol), cyclopropylamine (74 mg, 1.3 mmol), BOP (86 mg, 0.19 mmol) and DIEA (34 □L, 0.19 mmol) in DMF (2.5 mL) was stirred at room temperature overnight. The reaction mixture was poured into saturated aqueous sodium bicarbonate solution and extracted with ethyl acetate. The organic phase was dried over sodium sulfate, filtered and concentrated to dryness. The residue was purified by RP HPLC using a C18 column with a gradient of H₂O, 0.05% TFA-acetonitrile, to provide 4.5 mg (5% yield) of the title compound which was characterised as follows:

MS (m/z) 573.2 [M+H]⁺;

HPLC Rₜ=1.96 min.; and

¹H-NMR (300 MHz, CD₃OD) δ 0.62 (2H, m), 0.78 (2H, m), 2.83 (1H, m), 4.33 (2H, m), 4.85 (2H, s), 7.53 (2H, d), 7.86 (2H, d), 7.94 (2H, d), 8.32 (2H, d) and 9.39 (1H, s) ppm.

### Example I

### Synthesis of 4-{[6-(3-Chloro-4-fluoro-phenyl)-4-(2,2,2-trifluoro-ethylamino)-pteridin-2-ylamino]-methyl}-benzenesulfonamide

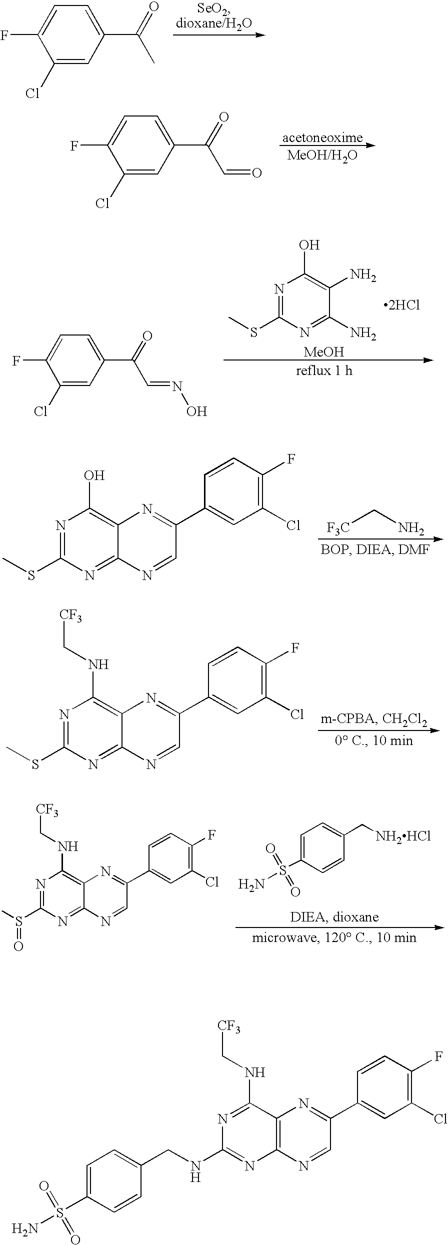

### (3-Chloro-4-fluoro-phenyl)-oxo-acetaldehyde oxime

The procedure for the synthesis of 4-(2-Hydroxyimino-acetyl)-benzoic acid ethyl ester was repeated, except for the use 1-(3-Chloro-4-fluoro-phenyl)-ethanone instead of 4-Acetyl-benzoic acid ethyl ester. The title compound was characterised as follows:

MS (m/z) 203.7 [M+H]⁺;

HPLC Rₜ=2.26 min.; and

¹H-NMR (300 MHz, DMSO-d₆) δ 7.53 (1H, dd), 7.91 (1H, dd), 7.95 (1H, s) and 8.13 (1H, dd) ppm.

### 6-(3-Chloro-4-fluoro-phenyl)-2-methylsulfanyl-pteridin-4-ol

The procedure for the synthesis of 4-(4-Hydroxy-2-methylsulfanyl-pteridin-6-yl)-benzoic acid ethyl ester was repeated, except for the use of (3-Chloro-4-fluoro-phenyl)-oxo-acetaldehyde oxime instead of 4-(2-Hydroxyimino-acetyl)-benzoic acid ethyl ester. The title compound was characterised as follows:

MS (m/z) 323.1 [M+H]⁺;

HPLC Rₜ=2.23 min; and

¹H-NMR (300 MHz, DMSO-d₆) δ 2.57 (3H, s), 7.57 (1H, dd), 8.20 (1H, m), 8.36 (1H, dd) and 9.49 (1H, s) ppm.

### [6-(3-Chloro-4-fluoro-phenyl)-2-methylsulfanyl-pteridin-4-yl]-(2,2,2-trifluoro-ethyl)-amine

The procedure for the synthesis of 4-[2-Methylsulfanyl-4-(2,2,2-trifluoro-ethylamino)-pteridin-6-yl]-benzoic acid ethyl ester was repeated, except for the use of 6-(3-Chloro-4-fluoro-phenyl)-2-methylsulfanyl-pteridin-4-ol instead of 4-(4-Hydroxy-2-methylsulfanyl-pteridin-6-yl)-benzoic acid ethyl ester. The title compound was characterised as follows:

MS (m/z) 404.1 [M+H]⁺; and

HPLC Rₜ=2.84 min.

### [6-(3-Chloro-4-fluoro-phenyl)-2-methanesulfinyl-pteridin-4-yl]-(2,2,2-trifluoro-ethyl)-amine

The procedure for the synthesis of [6-(4-Fluoro-phenyl)-2-methanesulfinyl-pteridin-4-yl]-(2,2,2-trifluoro-ethyl)-amine was repeated, except for the use of [6-(3-Chloro-4-fluoro-phenyl)-2-methylsulfanyl-pteridin-4-yl]-(2,2,2-trifluoro-ethyl)-amine instead of [6-(4-Fluoro-phenyl)-2-methylsulfanyl-pteridin-4-yl]-(2,2,2-trifluoro-ethyl)-amine. The title compound was characterised as follows:

MS (m/z) 420.1 [M+H]⁺; and

HPLC Rₜ=2.36 min.

### 4-{[6-(3-Chloro-4-fluoro-phenyl)-4-(2,2,2-trifluoro-ethylamino)-pteridin-2-ylamino]-methyl}-benzenesulfonamide

The procedure for the synthesis of 4-{[6-(4-Fluoro-phenyl)-4-oxo-3,4-dihydro-pteridin-2-ylamino]-methyl}-benzenesulfonamide was repeated, except for the use of [6-(3-Chloro-4-fluoro-phenyl)-2-methanesulfinyl-pteridin-4-yl]-(2,2,2-trifluoro-ethyl)-amine instead of 6-(4-Fluoro-phenyl)-2-methyl-sulfinyl-3H-pteridin-4-one. The title compound was characterised as follows:

MS (m/z) 542.2 [M+H]⁺;

HPLC Rₜ=2.29 min.; and

¹H-NMR (300 MHz, CD₃OD) δ 4.36 (2H, q), 4.86 (2H, s), 7.38 (1H, t), 7.54 (2H, d), 7.86 (2H, d), 8.20 (1H, m), 8.52 (1H, d) and 9.34 (1H, s) ppm.

### Example J

### Anti-HCV Assay/Replicon Assay

The anti HCV activity of specifically 2,4,6-trisubstituted pteridine derivatives was tested in a human hepatoma Huh-7 cell line harbouring a HCV replicon. The assay comprised the following steps:

Step 1: compound preparation and serial dilution involved the following alternatives, depending upon the water solubility of pteridine derivative being tested:
1. with respect to water soluble 2,4,6-trisubstituted pteridine derivatives, 500 μL of solution in cell media (DMEM, 10% FBS, P/S, L-Glutamine) was prepared with a concentration that is twice the concentration of the starting final serial dilution concentration. 150 μL of the solution was added to the pre-specified wells in column 1 of a 96-well cell culture plate (PerkinElmer, white plate, cat. #6005181, for EC50 assay; black plate, cat. #6005182 for CC50 assay). The rest of the plate, i.e. columns 2-12, was filled with 100 μL of cell media. The plate was then placed on a Precision 2000 Workstation in order to start serial dilution. Pteridine compounds were diluted three times each step from column 1 to column 10. Column 11 was used as a blank control (no pteridine compound added).
2. With respect to poorly water soluble pteridine derivatives, DMSO was used as a solvent, and serial dilution was performed in 50% DMSO in water, in a 384-well plate. A solution containing the relevant compound at 100-fold the concentration of the starting final serial dilution concentration was prepared in 50% DMSO in water and added to the pre-specified wells in column 1 of a polypropylene 384-well plate. This plate was then placed on a Precision 2000 Workstation in order to start serial dilution. After serial dilution, 2 μL of the solution was transferred from the 384-well plate to a 96-well cell culture plate containing 100 μL of cell media on a Biomek FX Workstation. The DMSO concentration in the final assay condition was 0.5% after cells have been added to the plate and the total volume in each well was brought to 200 μL.
   Step 2: to each well of the serial dilution plate prepared in step 1, 100 μL of cell media containing 6000 suspended Huh-7 HCV replicon cells was added with a Multidrop workstation. Plates were incubated for 3 days at 37° C. with 5% CO₂.
   Step 3: detection:
1) with respect to the EC₅₀ assay, the media in a 96-well cell culture plate was aspirated with a Biotek EL405 plate-washer. A volume of 200 μL of a solution containing a 1:1 mixture of a cell-lysis buffer (commercially available from Promega, Luciferase Cell Culture Lysis 5× Reagent, cat. #E1531) and a luciferase substrate solution (commercially available from Promega, Luciferase Assay, cat. # E4550) was added to each well of the plate with Multidrop. The plate was incubated for 30 minutes at room temperature before the luminescence signal was measured with a TopCount plate-reader.
2) with respect to the CC₅₀ assay, 100 μL of a pre-mixed CellTiter-Glo (commercially available from Promega, cat. # G7572) solution was added directly to the cell culture in each well of the plate and the luminescence signal was measured with a TopCount plate-reader after 10 minutes of incubation at room temperature.

Table 1, Table 2 and Table 3 respectively show EC₅₀ (expressed in nM, i.e. nmole/l) and CC₅₀ values (expressed in μM, i.e. μmole/l) determined in the above assay for 2,4,6-trisubstituted pteridine derivatives known in the art as well as for those of examples A and B.

Results are expressed by the following data:
the 50% effective concentration (EC₅₀), i.e. the concentration that protects 50% of the cell monolayer from virus-induced cythopathic effect, and
the 50% cytostatic concentration (CC₅₀), i.e. the concentration that results in 50% inhibition of cell growth.

All tables also present values of the CC₅₀/EC₅₀ ratio, which is indicative of the selective activity of the tested compounds with respect to the virus. In each table, the first column indicates the example No. of this invention, the second column indicates the example No. of the relevant 2,4,6-trisubstituted pteridine in the patent document where said compound was previously disclosed. The following columns indicate the type of substituents present in positions 2, 4 and 6 of the pteridine scaffold respectively.

The following abbreviations have been used in the description of these substituents:
Ph=phenyl,
Me=methyl,
Et=ethyl,
Pr=n-propyl,
iPr=isopropyl,
nBu=n-butyl,
tBu=tert-butyl,
BOC=tert-butyloxycarbonyl, and
(S) and (R) refer to the configuration of the stereocenter.

TABLE 1
anti-HCV activity of pteridine compounds disclosed in WO 2005/039587

| | | | | | EC₅₀ | CC₅₀ |
|---|---|---|---|---|---|---|
| | | | | | (A < 0.3 μM; | (A < 10 μM; B |
| | | | | | <0.3 μM < B < 1 μM; | 10-20 μM; C > |
| Ex. | WO | 2-substituent | 4-substituent | 6-substituent | 1 μM < C < 10 μM) | 20 μM) |
| 1 | 17 | NH₂ | (4-COCH₂OPh)piperazinyl | (3,4-diOMe)Ph | A | C |
| 2 | 154 | NH₂ | (4-CH₂CH₂OPh)piperazinyl | (3,4-diOMe)Ph | A | C |
| 3 | 103 | NH₂ | (4-CONH(3-Me)Ph)piperazinyl | (3,4-diOMe)Ph | A | A |
| 12 | 80 | NHCH₂-2-thienyl | NH₂ | (3,4-diOMe)Ph | B | B |
| 16 | 10 | NH₂ | (4-COCH₂CH₂CH₂CH₂CH₃)piperazinyl | (3,4-diOMe)Ph | A | B |
| 19 | 13 | NH₂ | (4-CO-2-thienyl)piperazinyl | (3,4-diOMe)Ph | A | B |
| 20 | 14 | NH₂ | (4-CONEt₂)piperazinyl | (3,4-diOMe)Ph | A | C |
| 22 | 19 | NH₂ | (4-CO-4-pyridyl)piperazinyl | (3,4-diOMe)Ph | A | B |
| 24 | 21 | NH₂ | (4-CO(4-O-n-pentyl)Ph)piperazinyl | (3,4-diOMe)Ph | A | A |
| 26 | 31 | NH₂ | (4-SO₂-Ph)piperazinyl | (3,4-diOMe)Ph | A | B |
| 27 | 36 | NH₂ | (4-COCH₂-2-thienyl)piperazinyl | (3,4-diOMe)Ph | A | A |
| 28 | 37 | NH₂ | (4-COCHCH(E)Ph)piperazinyl | (3,4-diOMe)Ph | A | A |
| 29 | 38 | NH₂ | (4-CO-1-pyrrolidinyl)piperazinyl | (3,4-diOMe)Ph | A | B |
| 31 | 40 | NH₂ | (4-CO-3-(2,6-di-Cl-5-F)pyridyl) piperazinyl | (3,4-diOMe)Ph | A | A |
| 32 | 41 | NH₂ | (4-COCH₂OMe)piperazinyl | (3,4-diOMe)Ph | A | C |
| 34 | 43 | NH₂ | (4-SO₂CH₂Ph)piperazinyl | (3,4-diOMe)Ph | A | C |
| 36 | 45 | NH₂ | (4-COCH₂(4-Cl)Ph)piperazinyl | (3,4-diOMe)Ph | A | A |
| 38 | 47 | NH₂ | (4-CO-3-furanyl)piperazinyl | (3,4-diOMe)Ph | B | B |
| 43 | 48 | NH₂ | (4-CO₂CH₂Ph)piperazinyl | (3,4-diOMe)Ph | A | A |
| 49 | 136 | NH₂ | (4-CH₂(3-Me)Ph)piperazinyl | (3,4-diOMe)Ph | A | B |
| 52 | 140 | NH₂ | (4-CH₂(4-Me)Ph)piperazinyl | (3,4-diOMe)Ph | B | C |
| 53 | 141 | NH₂ | (4-CH₂(2-F)Ph)piperazinyl | (3,4-diOMe)Ph | A | A |
| 54 | 18 | NH₂ | (4-CO(4-n-Bu)Ph)piperazinyl | (3,4-diOMe)Ph | A | A |
| 55 | 122 | NH₂ | (4-CH₂CH₂OMe)piperazinyl | (3,4-diOMe)Ph | B | C |
| 59 | 90 | NH₂ | (4-CO₂(4-Me)Ph)piperazinyl | (3,4-diOMe)Ph | A | A |
| 60 | 91 | NH₂ | (4-CO₂(4-OMe)Ph)piperazinyl | (3,4-diOMe)Ph | A | A |
| 63 | 94 | NH₂ | (4-CO₂(4-Cl)Ph)piperazinyl | (3,4-diOMe)Ph | A | A |
| 65 | 97 | NH₂ | (4-CO₂CH₂CH₂OMe)piperazinyl | (3,4-diOMe)Ph | A | B |
| 73 | 152 | NH₂ | (4-CH(Me)Ph)piperazinyl | (3,4-diOMe)Ph | A | A |
| 80 | 156 | NH₂ | (4-CH₂-3-pyridyl)piperazinyl | (3,4-diOMe)Ph | A | B |
| 81 | 157 | NH₂ | (4-CH₂-4-pyridyl)piperazinyl | (3,4-diOMe)Ph | B | B |
| 83 | 130 | NH₂ | (4-CH₂CO-morpholinyl)piperazinyl | (3,4-diOMe)Ph | B | C |
| 84 | 131 | NH₂ | (4-CH₂CO-pyrrolidinyl)piperazinyl | (3,4-diOMe)Ph | A | C |
| 85 | 132 | NH₂ | (4-CH₂CON(Me)Ph)piperazinyl | (3,4-diOMe)Ph | A | A |
| 86 | 159 | NH₂ | (4-CH₂CH₂Ph)piperazinyl | (3,4-diOMe)Ph | A | B |
| 87 | 99 | NH₂ | (4-CONHPh)piperazinyl | (3,4-diOMe)Ph | A | A |
| 88 | 100 | NH₂ | (4-CONH(4-F)Ph)piperazinyl | (3,4-diOMe)Ph | A | A |
| 89 | 102 | NH₂ | (4-CONH(4-CN)Ph)piperazinyl | (3,4-diOMe)Ph | A | C |
| 95 | 161 | NH₂ | (4-(4-OMe)Ph)piperazinyl | (3,4-diOMe)Ph | B | C |
| 98 | 135 | NH₂ | (4-CH₂CO₂Et)piperazinyl | (3,4-diOMe)Ph | A | B |
| 99 | 177 | NH₂ | (4-CH₂-2-pyridyl)piperazinyl | (3,4-diOMe)Ph | B | B |
| 100 | 163 | NH₂ | (4-CH₂CH₂CN)piperazinyl | (3,4-diOMe)Ph | A | B |
| 104 | 165 | NH₂ | (4-CH₂CH₂OEt)piperazinyl | (3,4-diOMe)Ph | A | C |
| 108 | 169 | NH₂ | (4-CH₂-2-tetrahydrofuranyl)piperazinyl | (3,4-diOMe)Ph | B | C |
| 109 | 170 | NH₂ | (4-CH₂CH(—OCH₂CH₂O—))piperazinyl | (3,4-diOMe)Ph | B | C |
| 111 | 104 | NH₂ | (4-CONHCH₂Ph)piperazinyl | (3,4-diOMe)Ph | A | B |
| 112 | 105 | NH₂ | (4-CONHCH₂(4-F)Ph)piperazinyl | (3,4-diOMe)Ph | A | A |
| 113 | 106 | NH₂ | (4-CONH(3-Cl-4-F)Ph)piperazinyl | (3,4-diOMe)Ph | A | A |
| 114 | 107 | NH₂ | (4-CONH-2-thienyl)piperazinyl | (3,4-diOMe)Ph | A | A |
| 115 | 108 | NH₂ | (4-CONHCH₂CH₂-2-thienyl)piperazinyl | (3,4-diOMe)Ph | A | A |
| 118 | 109 | NH₂ | (4-CONH-nBu)piperazinyl | (3,4-diOMe)Ph | A | A |
| 119 | 117 | NH₂ | (4-CON(Me)Ph)piperazinyl | (3,4-diOMe)Ph | A | A |
| 121 | 118 | NH₂ | (4-CON(Et)Ph)piperazinyl | (3,4-diOMe)Ph | A | A |
| 122 | | NH₂ | (4-CONH(3-Cl)Ph)piperazinyl | (3,4-diOMe)Ph | A | A |
| 123 | | NH₂ | (4-CONH(3-CF₃)Ph)piperazinyl | (3,4-diOMe)Ph | A | A |
| 124 | | NH₂ | (4-CONH(4-Br)Ph)piperazinyl | (3,4-diOMe)Ph | A | A |

(end of table 1)

TABLE 2
anti-HCV activity of pteridine compounds disclosed in US 2004/0077859

| | | | | | EC50 | CC50 |
|---|---|---|---|---|---|---|
| | | | | | (A < 0.3 μM; | (A < 10 μM; B |
| | | | | | <0.3 μM < B < 1 μM; | 10-20 μM; C > |
| Ex. | US | 2-substituent | 4-substituent | 6-substituent | 1 μM < C < 10 μM) | 20 μM) |
| 131 | 77 | NH₂ | OEt | (4-F)Ph | A | C |
| 134 | 52 | NH₂ | OEt | (3,4-diOMe)Ph | A | C |
| 158 | 24 | NH₂ | Morpholinyl | (3,4-diOMe)Ph | A | B |
| 181 | 32 | NH₂ | (4-Me)piperazinyl | (3,4-diOMe)Ph | A | B |

(end of Table 2)

TABLE 3
anti-HCV activity of pteridine compounds disclosed in WO 2005/021003

| | | | | | EC50 | CC50 |
|---|---|---|---|---|---|---|
| | | | | | (A < 0.3 μM; | (A < 10 μM; B |
| | | | | | <0.3 μM < B < 1 μM; | 10-20 μM; C > |
| Ex. | WO | 2-substituent | 4-substituent | 6-substituent | 1 μM < C < 10 μM) | 20 μM) |
| 201 | 97 | NH₂ | Morpholinyl | (3-NHSO₂Et)Ph | B | C |
| 216 | 78 | NH₂ | Morpholinyl | (4-NHCOCH₂CH₃)Ph | B | C |
| 230 | 119 | NH₂ | NHCH(Me)(R)CH₂CH₃ | (3,4-diOMe)Ph | B | C |
| 231 | 74 | NH₂ | Morpholinyl | (4-NH₂)Ph | B | C |
| 234 | 120 | NH₂ | NHCH(Me)(S)CH₂CH₃ | (3,4-diOMe)Ph | B | C |

TABLE 4
anti-HCV activity of further examples

| | EC50 | CC50 |
|---|---|---|
| | (A < 0.3 μM; <0.3 | (A < 10 μM; |
| | μM < B < 1 μM; | B 10-20 μM; |
| Ex. | 1 μM < C < 10 μM) | C > 20 μM) |
| C | A | C |
| D | A | C |
| E | A | A |
| F | A | A |
| G | A | C |
| H | B | C |
| I | B | B |

### Example K

### Anti-HIV Assay

The anti HIV activity of some of the pteridine compounds listed in example C was tested in MT-4 cells infected with HIV IIIb strain. The assay comprised the following steps:
Step 1 (sample preparation). The compound stock solutions as prepared for Example C were diluted in MT4 cell media at a concentration equal to twice the concentration of the HCV EC50 or CC50 (the highest concentration of the two was chosen) obtained in Example C. To pre-defined wells of a 384-well black cell culture plate (NUNC), a volume of 50 μL of the compound solution is added. Serial dilution is performed by the workstation across the plate. Each compound is serially diluted 5-fold in each step, for a total of 6 steps (7 total concentrations). Compounds are tested in triplicates within each plate. Negative control was performed using 40 μL cell media only. At the end of the serial dilution, each well on the plate contained 40 μL of media containing different concentrations of compounds. A volume of 20 μL was transferred to a new plate to duplicate the plate.
step 2 (starting the assay): the plates are transferred into the HIV laboratory. To each plate, a volume of 20 μL MT-4 cell suspension (105 cells/mL) is dispensed into each well of the plate with the Multidrop dispenser. The HIV IIIb stock solution was diluted 1 to 500 in cell media (2 μL of virus+998 μL of media). 100-120 μL of diluted virus was then added to 10 ml cell suspension (containing 106 MT-4 cells). 20 μL of this cell suspension/virus was dispensed to each well of the plates with the Multidrop dispenser. The plates were incubated at 37° C. for 5 days in the cell culture incubator in the HIV laboratory.
step 3 (detection): on day 5, 40 μL of the pre-prepared CellTiter-Glo reagent was added to each well of the plates using the Multidrop dispenser. The plates were incubated at room temperature for 10 minutes to stabilize the luminescence signal. The luminescence was measured on the Victor 2 plate reader with an integration time of 0.25-1 seconds per sample.

Table 5 below shows the anti-HIV 50% effective concentration (EC50 value, expressed in μM, i.e. μmole/L) determined in the above assay. It is clear from the table that none of the pteridine compounds tested is active against HIV.

TABLE 5

| | 2- | 4- | 6- | EC50 |
|---|---|---|---|---|
| Ex. | substituent | substituent | substituent | (μM) |
| 134 | NH2 | OEt | (3,4-diOMe)Ph | >100 |
| 158 | NH2 | morpholinyl | (3,4-diOMe)Ph | >100 |
| 181 | NH2 | (4-Me)piperazinyl | (3,4-diOMe)Ph | >100 |
| 201 | NH2 | morpholinyl | (3-NHSO2Et)Ph | 17.8 |
| 216 | NH2 | morpholinyl | (4-NHCOCH2CH3)Ph | >100 |

### Example L

### Synthesis and anti-HCV activity of 2-amino-4-isopropoxy-6-(4-fluorophenyl)-pteridine

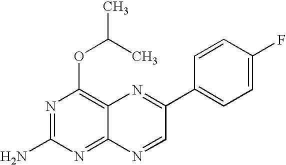

To a degassed solution of the compound of 2-amino-6-chloro-4-isopropoxypteridine (described in WO 2005/021003 as example 12) in THF was added a degassed solution of sodium carbonate (0.4 M solution in water), tetrakis(triphenylphosphine) palladium and 4-fluorophenylboronic acid. The solution was refluxed for 4 hours. Solvents were concentrated in vacuo and the residue was purified by flash chromatography (silica) with an appropriate CH₃OH/CH₂Cl₂ mixture (2:98 or 3:97) as the eluent.

The anti-HCV activity of the title compound was measured in the assay of example J and the following results obtained are listed in Table 6.

TABLE 6

| | EC50 | CC50 |
|---|---|---|
| | (A < 0.3 μM; <0.3 | (A < 10 μM; |
| | μM < B < 1 μM; | B 10-20 μM; |
| Ex. | 1 μM < C < 10 μM) | C > 20 μM) |
| 2-amino-4-isopropoxy-6- | A | C |
| (4-fluorophenyl)-pteridine | | |

### Examples 300 to 460

### Synthesis of 4-ethoxy-6-(4-fluorophenyl)-pteridin-2-yl]-(4-fluoro-benzyl)-amine Analogues

The procedure of the second step of example C is repeated while replacing p-fluorophenylboronic acid with an alternative boronic acid or pinacol ester thereof. In this way, the following analogues are obtained in similar yields:
4-ethoxy-6-(4-acetamidophenyl)-pteridin-2-yl]-(4-fluoro-benzyl)-amine,
4-ethoxy-6-(3-acetamidophenyl)-pteridin-2-yl]-(4-fluoro-benzyl)-amine,
4-ethoxy-6-(4-acetylphenyl)-pteridin-2-yl]-(4-fluoro-benzyl)-amine,
4-ethoxy-6-(3-acetylphenyl)-pteridin-2-yl]-(4-fluoro-benzyl)-amine,
4-ethoxy-6-(2-acetylphenyl)-pteridin-2-yl]-(4-fluoro-benzyl)-amine,
4-ethoxy-6-(5-acetyl-2-chlorophenyl)-pteridin-2-yl]-(4-fluoro-benzyl)-amine,
4-ethoxy-6-(4-acetyl-3-fluorophenyl)-pteridin-2-yl]-(4-fluoro-benzyl)-amine,
4-ethoxy-6-(5-acetyl-2-fluorophenyl)-pteridin-2-yl]-(4-fluoro-benzyl)-amine,
4-ethoxy-6-(3-aminophenyl)-pteridin-2-yl]-(4-fluoro-benzyl)-amine,
4-ethoxy-6-(4-aminomethylphenyl)-pteridin-2-yl]-(4-fluoro-benzyl)-amine,
4-ethoxy-6-(4-benzyloxy-2-fluorophenyl)-pteridin-2-yl]-(4-fluoro-benzyl)-amine,
4-ethoxy-6-(4-benzyloxy-3-fluorophenyl)-pteridin-2-yl]-(4-fluoro-benzyl)-amine,
4-ethoxy-6-(4-bromophenyl)-pteridin-2-yl]-(4-fluoro-benzyl)-amine,
4-ethoxy-6-(3-bromophenyl)-pteridin-2-yl]-(4-fluoro-benzyl)-amine,
4-ethoxy-6-(4-bromo-2,5-dimethylphenyl)-pteridin-2-yl]-(4-fluoro-benzyl)-amine,
4-ethoxy-6-(2-bromo-5-fluorophenyl)-pteridin-2-yl]-(4-fluoro-benzyl)-amine,
4-ethoxy-6-(2-bromo-6-fluorophenyl)-pteridin-2-yl]-(4-fluoro-benzyl)-amine,
4-ethoxy-6-(2-carboxy-5-fluorophenyl)-pteridin-2-yl]-(4-fluoro-benzyl)-amine,
4-ethoxy-6-(2-carboxyphenyl)-pteridin-2-yl]-(4-fluoro-benzyl)-amine,
4-ethoxy-6-(4-carboxyphenyl)-pteridin-2-yl]-(4-fluoro-benzyl)-amine,
4-ethoxy-6-(4-carboxy-2-chlorophenyl)-pteridin-2-yl]-(4-fluoro-benzyl)-amine,
4-ethoxy-6-(5-carboxy-2-chlorophenyl)-pteridin-2-yl]-(4-fluoro-benzyl)-amine,
4-ethoxy-6-(4-carboxy-3-chlorophenyl)-pteridin-2-yl]-(4-fluoro-benzyl)-amine,
4-ethoxy-6-(3-carboxyphenyl)-pteridin-2-yl]-(4-fluoro-benzyl)-amine,
4-ethoxy-6-(2-chloro-5-formylphenyl)-pteridin-2-yl]-(4-fluoro-benzyl)-amine,
4-ethoxy-6-(2-chloro-5-hydroxyphenyl)-pteridin-2-yl]-(4-fluoro-benzyl)-amine,
4-ethoxy-6-(3-chloro-4-fluorophenyl)-pteridin-2-yl]-(4-fluoro-benzyl)-amine,
4-ethoxy-6-(2-chloro-4-fluorophenyl)-pteridin-2-yl]-(4-fluoro-benzyl)-amine,
4-ethoxy-6-(4-chloro-2-fluorophenyl)-pteridin-2-yl]-(4-fluoro-benzyl)-amine,
4-ethoxy-6-(3-chloro-5-methoxyphenyl)-pteridin-2-yl]-(4-fluoro-benzyl)-amine,
4-ethoxy-6-(2-chloro-4-methylphenyl)-pteridin-2-yl]-(4-fluoro-benzyl)-amine,
4-ethoxy-6-(2-chloro-5-methylphenyl)-pteridin-2-yl]-(4-fluoro-benzyl)-amine,
4-ethoxy-6-(3-chloro-5-trifluoromethylphenyl)-pteridin-2-yl]-(4-fluoro-benzyl)-amine,
4-ethoxy-6-(2-chloro-5-trifluoromethoxyphenyl)-pteridin-2-yl]-(4-fluoro-benzyl)-amine,
4-ethoxy-6-(4-chloro-2-trifluoromethylphenyl)-pteridin-2-yl]-(4-fluoro-benzyl)-amine,
4-ethoxy-6-(4-chlorophenyl)-pteridin-2-yl]-(4-fluoro-benzyl)-amine,
4-ethoxy-6-(3-chlorophenyl)-pteridin-2-yl]-(4-fluoro-benzyl)-amine,
4-ethoxy-6-(2-chlorophenyl)-pteridin-2-yl]-(4-fluoro-benzyl)-amine,
4-ethoxy-6-(4-cyanophenyl)-pteridin-2-yl]-(4-fluoro-benzyl)-amine,
4-ethoxy-6-(3-cyanophenyl)-pteridin-2-yl]-(4-fluoro-benzyl)-amine,
4-ethoxy-6-(2-cyanophenyl)-pteridin-2-yl]-(4-fluoro-benzyl)-amine,
4-ethoxy-6-(2,6-dichlorophenyl)-pteridin-2-yl]-(4-fluoro-benzyl)-amine,
4-ethoxy-6-(3,4-dichlorophenyl)-pteridin-2-yl]-(4-fluoro-benzyl)-amine,
4-ethoxy-6-(2,6-dichlorophenyl)-pteridin-2-yl]-(4-fluoro-benzyl)-amine,
4-ethoxy-6-(2,4-dichlorophenyl)-pteridin-2-yl]-(4-fluoro-benzyl)-amine,
4-ethoxy-6-(2,3-dichlorophenyl)-pteridin-2-yl]-(4-fluoro-benzyl)-amine,
4-ethoxy-6-(3,5-dichlorophenyl)-pteridin-2-yl]-(4-fluoro-benzyl)-amine,
4-ethoxy-6-(3,5-difluorophenyl)-pteridin-2-yl]-(4-fluoro-benzyl)-amine,
4-ethoxy-6-(3,4-difluorophenyl)-pteridin-2-yl]-(4-fluoro-benzyl)-amine,
4-ethoxy-6-(2,6-difluorophenyl)-pteridin-2-yl]-(4-fluoro-benzyl)-amine,
4-ethoxy-6-(2,5-difluorophenyl)-pteridin-2-yl]-(4-fluoro-benzyl)-amine,
4-ethoxy-6-(2,4-difluorophenyl)-pteridin-2-yl]-(4-fluoro-benzyl)-amine,
4-ethoxy-6-(2,3-difluorophenyl)-pteridin-2-yl]-(4-fluoro-benzyl)-amine,
4-ethoxy-6-(4-[N,N-dimethylamino]phenyl)-pteridin-2-yl]-(4-fluoro-benzyl)-amine,
4-ethoxy-6-(2-[N,N-dimethylaminomethyl]phenyl)-pteridin-2-yl]-(4-fluoro-benzyl)-amine,
4-ethoxy-6-(3,5-dimethylphenyl)-pteridin-2-yl]-(4-fluorobenzyl)-amine,
4-ethoxy-6-(3,4-dimethylphenyl)-pteridin-2-yl]-(4-fluorobenzyl)-amine,
4-ethoxy-6-(2,6-dimethylphenyl)-pteridin-2-yl]-(4-fluorobenzyl)-amine,
4-ethoxy-6-(2,6-dimethoxyphenyl)-pteridin-2-yl]-(4-fluorobenzyl)-amine,
4-ethoxy-6-(2,5-dimethoxyphenyl)-pteridin-2-yl]-(4-fluorobenzyl)-amine,
4-ethoxy-6-(2,4-dimethoxyphenyl)-pteridin-2-yl]-(4-fluorobenzyl)-amine,
4-ethoxy-6-(4-ethoxyphenyl)-pteridin-2-yl]-(4-fluorobenzyl)-amine,
4-ethoxy-6-(2-ethoxyphenyl)-pteridin-2-yl]-(4-fluorobenzyl)-amine,
4-ethoxy-6-(4-ethoxycarbonylphenyl)-pteridin-2-yl]-(4-fluorobenzyl)-amine,
4-ethoxy-6-(3-ethoxycarbonylphenyl)-pteridin-2-yl]-(4-fluorobenzyl)-amine,
4-ethoxy-6-(4-ethylphenyl)-pteridin-2-yl]-(4-fluorobenzyl)-amine,
4-ethoxy-6-(4-fluorophenyl)-pteridin-2-yl]-(4-fluorobenzyl)-amine,
4-ethoxy-6-(3-fluorophenyl)-pteridin-2-yl]-(4-fluorobenzyl)-amine,
4-ethoxy-6-(2-fluorophenyl)-pteridin-2-yl]-(4-fluorobenzyl)-amine,
4-ethoxy-6-(3-fluoro-4-formylphenyl)-pteridin-2-yl]-(4-fluorobenzyl)-amine,
4-ethoxy-6-(4-fluoro-3-formylphenyl)-pteridin-2-yl]-(4-fluorobenzyl)-amine,
4-ethoxy-6-(4-fluoro-2-methylphenyl)-pteridin-2-yl]-(4-fluorobenzyl)-amine,
4-ethoxy-6-(2-fluoro-5-methylphenyl)-pteridin-2-yl]-(4-fluorobenzyl)-amine,
4-ethoxy-6-(2-fluoro-5-methoxyphenyl)-pteridin-2-yl]-(4-fluorobenzyl)-amine,
4-ethoxy-6-(5-fluoro-2-methoxycarbonylphenyl)-pteridin-2-yl]-(4-fluorobenzyl)-amine,
4-ethoxy-6-(2-formyl-5-methoxyphenyl)-pteridin-2-yl]-(4-fluorobenzyl)-amine,
4-ethoxy-6-(5-formyl-2-methoxyphenyl)-pteridin-2-yl]-(4-fluorobenzyl)-amine,
4-ethoxy-6-(2-formyl-5-methylphenyl)-pteridin-2-yl]-(4-fluorobenzyl)-amine,
4-ethoxy-6-(2-formylphenyl)-pteridin-2-yl]-(4-fluorobenzyl)-amine,
4-ethoxy-6-(3-formylphenyl)-pteridin-2-yl]-(4-fluorobenzyl)-amine,
4-ethoxy-6-(4-formylphenyl)-pteridin-2-yl]-(4-fluorobenzyl)-amine,
4-ethoxy-6-(3-hydroxy-4-methoxycarbonylphenyl)-pteridin-2-yl]-(4-fluorobenzyl)-amine,
4-ethoxy-6-(3-hydroxymethylphenyl)-pteridin-2-yl]-(4-fluorobenzyl)-amine,
4-ethoxy-6-(4-hydroxymethylphenyl)-pteridin-2-yl]-(4-fluorobenzyl)-amine,
4-ethoxy-6-(4-hydroxyphenyl)-pteridin-2-yl]-(4-fluorobenzyl)-amine,
4-ethoxy-6-(3-hydroxyphenyl)-pteridin-2-yl]-(4-fluorobenzyl)-amine,
4-ethoxy-6-(4-iodophenyl)-pteridin-2-yl]-(4-fluorobenzyl)-amine,
4-ethoxy-6-(3-iodophenyl)-pteridin-2-yl]-(4-fluorobenzyl)-amine,
4-ethoxy-6-(4-isopropoxycarbonylphenyl)-pteridin-2-yl]-(4-fluorobenzyl)-amine,
4-ethoxy-6-(3-isopropoxycarbonylphenyl)-pteridin-2-yl]-(4-fluorobenzyl)-amine,
4-ethoxy-6-(4-methanesulfonylphenyl)-pteridin-2-yl]-(4-fluorobenzyl)-amine,
4-ethoxy-6-(4-methoxy-2-formylphenyl)-pteridin-2-yl]-(4-fluorobenzyl)-amine,
4-ethoxy-6-(4-methoxycarbonylphenyl)-pteridin-2-yl]-(4-fluorobenzyl)-amine,
4-ethoxy-6-(3-methoxycarbonylphenyl)-pteridin-2-yl]-(4-fluorobenzyl)-amine,
4-ethoxy-6-(4-methoxyphenyl)-pteridin-2-yl]-(4-fluorobenzyl)-amine,
4-ethoxy-6-(3-methoxyphenyl)-pteridin-2-yl]-(4-fluorobenzyl)-amine,
4-ethoxy-6-(2-methoxyphenyl)-pteridin-2-yl]-(4-fluorobenzyl)-amine,
4-ethoxy-6-(3,4-methylenedioxyphenyl)-pteridin-2-yl]-(4-fluorobenzyl)-amine,
4-ethoxy-6-(4-methylphenyl)-pteridin-2-yl]-(4-fluorobenzyl)-amine,
4-ethoxy-6-(2-methylphenyl)-pteridin-2-yl]-(4-fluorobenzyl)-amine,
4-ethoxy-6-(4-(methylthio)phenyl)-pteridin-2-yl]-(4-fluorobenzyl)-amine,
4-ethoxy-6-(3-(methylthio)phenyl)-pteridin-2-yl]-(4-fluorobenzyl)-amine,
4-ethoxy-6-(4-morpholinophenyl)-pteridin-2-yl]-(4-fluorobenzyl)-amine,
4-ethoxy-6-(3-nitrophenyl)-pteridin-2-yl]-(4-fluorobenzyl)-amine,
4-ethoxy-6-(4-phenoxyphenyl)-pteridin-2-yl]-(4-fluorobenzyl)-amine,
4-ethoxy-6-(4-(tert-butoxycarbonyl)phenyl)-pteridin-2-yl]-(4-fluoro-benzyl)-amine,
4-ethoxy-6-(3-(tert-butoxycarbonyl)phenyl)-pteridin-2-yl]-(4-fluoro-benzyl)-amine,
4-ethoxy-6-(2-(tert-butoxycarbonyl)phenyl)-pteridin-2-yl]-(4-fluoro-benzyl)-amine,
4-ethoxy-6-(4-(tert-butoxycarbonylamino)-3-methoxyphenyl)-pteridin-2-yl]-(4-fluorobenzyl)-amine,
4-ethoxy-6-(4-(tert-butylphenyl)-pteridin-2-yl]-(4-fluorobenzyl)-amine,
4-ethoxy-6-(4-(2-thienyl)phenyl)-pteridin-2-yl]-(4-fluorobenzyl)-amine,
4-ethoxy-6-(4-trifluoromethoxyphenyl)-pteridin-2-yl]-(4-fluorobenzyl)-amine,
4-ethoxy-6-(2,4,6-trimethylphenyl)-pteridin-2-yl]-(4-fluorobenzyl)-amine,
4-ethoxy-6-(3,4,5-trifluorophenyl)-pteridin-2-yl]-(4-fluorobenzyl)-amine,
4-ethoxy-6-(3-trifluoromethoxyphenyl)-pteridin-2-yl]-(4-fluorobenzyl)-amine,
4-ethoxy-6-(3-trifluoromethylphenyl)-pteridin-2-yl]-(4-fluorobenzyl)-amine,
4-ethoxy-6-(2-trifluoromethylphenyl)-pteridin-2-yl]-(4-fluorobenzyl)-amine,
4-ethoxy-6-(3,4,5-trimethoxyphenyl)-pteridin-2-yl]-(4-fluorobenzyl)-amine,
4-ethoxy-6-(4-vinylphenyl)-pteridin-2-yl]-(4-fluorobenzyl)-amine,
4-ethoxy-6-(2-acetamidopyridin-5-yl)-pteridin-2-yl]-(4-fluorobenzyl)-amine,
4-ethoxy-6-(2-benzothienyl)-pteridin-2-yl]-(4-fluorobenzyl)-amine,
4-ethoxy-6-(1-benzothiophen-3-yl)-pteridin-2-yl]-(4-fluorobenzyl)-amine,
4-ethoxy-6-(1-benzothiophen-2-yl)-pteridin-2-yl]-(4-fluorobenzyl)-amine,
4-ethoxy-6-(2-bromo-3-chloropyridin-4-yl)-pteridin-2-yl]-(4-fluoro-benzyl)-amine,
4-ethoxy-6-(2-bromo-3-methylpyridin-5-yl)-pteridin-2-yl]-(4-fluoro-benzyl)-amine,
4-ethoxy-6-(2-bromopyridin-5-yl)-pteridin-2-yl]-(4-fluorobenzyl)-amine,
4-ethoxy-6-(2-chloro-3-methylpyridin-5-yl)-pteridin-2-yl]-(4-fluoro-benzyl)-amine,
4-ethoxy-6-(2-chloro-6-isopropylpyridin-3-yl)-pteridin-2-yl]-(4-fluoro-benzyl)-amine,
4-ethoxy-6-(5-chlorothien-2-yl)-pteridin-2-yl]-(4-fluoro-benzyl)-amine,
4-ethoxy-6-(5-bromothien-2-yl)-pteridin-2-yl]-(4-fluoro-benzyl)-amine,
4-ethoxy-6-(2-chloro-3-fluoropyridin-4-yl)-pteridin-2-yl]-(4-fluoro-benzyl)-amine,
4-ethoxy-6-(2,5-dibromopyridin-3-yl)-pteridin-2-yl]-(4-fluoro-benzyl)-amine,
4-ethoxy-6-(2,6-dichloropyridin-3-yl)-pteridin-2-yl]-(4-fluoro-benzyl)-amine,
4-ethoxy-6-(2,4-dimethoxypyrimidin-5-yl)-pteridin-2-yl]-(4-fluoro-benzyl)-amine,
4-ethoxy-6-(3,5-dimethylisoxazol-4-yl)-pteridin-2-yl]-(4-fluoro-benzyl)-amine,
4-ethoxy-6-(2,4-dioxo-1,2,3,4-tetrahydropyrimidin-5-yl)-pteridin-2-yl]-(4-fluoro-benzyl)-amine,
4-ethoxy-6-(2-ethoxypyridin-3-yl)-pteridin-2-yl]-(4-fluorobenzyl)-amine,
4-ethoxy-6-(2-fluoro-3-methylpyridin-5-yl)-pteridin-2-yl]-(4-fluorobenzyl)-amine,
4-ethoxy-6-(2-fluoropyridin-3-yl)-pteridin-2-yl]-(4-fluorobenzyl)-amine,
4-ethoxy-6-(2-fluoropyridin-5-yl)-pteridin-2-yl]-(4-fluorobenzyl)-amine,
4-ethoxy-6-(5-formyl-2-furyl)-pteridin-2-yl]-(4-fluorobenzyl)-amine,
4-ethoxy-6-(5-formyl-thiophen-2-yl)-pteridin-2-yl]-(4-fluorobenzyl)-amine,
4-ethoxy-6-(furan-3-yl)-pteridin-2-yl]-(4-fluorobenzyl)-amine,
4-ethoxy-6-(furan-2-yl)-pteridin-2-yl]-(4-fluorobenzyl)-amine,
4-ethoxy-6-(indol-5-yl)-pteridin-2-yl]-(4-fluorobenzyl)-amine,
4-ethoxy-6-(isoquinolin-4-yl)-pteridin-2-yl]-(4-fluorobenzyl)-amine,
4-ethoxy-6-(5-methylfuran-2-yl)-pteridin-2-yl]-(4-fluorobenzyl)-amine,
4-ethoxy-6-(methoxypyrimidin-5-yl)-pteridin-2-yl]-(4-fluorobenzyl)-amine,
4-ethoxy-6-(5-methyl-1-benzothiophen-2-yl)-pteridin-2-yl]-(4-fluorobenzyl)-amine,
4-ethoxy-6-(5-methyl-3-phenyl-4-isoxazolyl)-pteridin-2-yl]-(4-fluorobenzyl)-amine,
4-ethoxy-6-(3-methylpyridin-2-yl)-pteridin-2-yl]-(4-fluorobenzyl)-amine,
4-ethoxy-6-(5-methylpyridin-2-yl)-pteridin-2-yl]-(4-fluorobenzyl)-amine,
4-ethoxy-6-(5-methylpyridin-3-yl)-pteridin-2-yl]-(4-fluorobenzyl)-amine,
4-ethoxy-6-(2-methoxypyridin-3-yl)-pteridin-2-yl]-(4-fluorobenzyl)-amine,
4-ethoxy-6-(2-methoxypyridin-5-yl)-pteridin-2-yl]-(4-fluorobenzyl)-amine,
4-ethoxy-6-(pyridin-4-yl)-pteridin-2-yl]-(4-fluorobenzyl)-amine,
4-ethoxy-6-(pyridin-3-yl)-pteridin-2-yl]-(4-fluorobenzyl)-amine,
4-ethoxy-6-(pyrimidin-5-yl)-pteridin-2-yl]-(4-fluorobenzyl)-amine,
4-ethoxy-6-(quinolin-3-yl)-pteridin-2-yl]-(4-fluorobenzyl)-amine,
4-ethoxy-6-(quinolin-8-yl)-pteridin-2-yl]-(4-fluorobenzyl)-amine, and
4-ethoxy-6-(thien-2-yl)-pteridin-2-yl]-(4-fluorobenzyl)-amine.

### Examples 500 to 661

### Synthesis of 4-isopropoxy-6-(4-fluorophenyl)-pteridin-2-yl]-(4-fluorobenzyl)-amine and Analogues Thereof

The procedure of example C is repeated, except that the first step is carried out starting from 6-chloro-4-ethoxy-pteridin-2-ylamine and that optionally p-fluorophenylboronic acid is replaced in the second step with an alternative boronic acid or pinacol ester thereof. In this way, the title compound and the following analogues are obtained in similar yields:
4-isopropoxy-6-(4-acetamidophenyl)-pteridin-2-yl]-(4-fluoro-benzyl)-amine,
4-isopropoxy-6-(3-acetamidophenyl)-pteridin-2-yl]-(4-fluoro-benzyl)-amine,
4-isopropoxy-6-(4-acetylphenyl)-pteridin-2-yl]-(4-fluoro-benzyl)-amine,
4-isopropoxy-6-(3-acetylphenyl)-pteridin-2-yl]-(4-fluoro-benzyl)-amine,
4-isopropoxy-6-(2-acetylphenyl)-pteridin-2-yl]-(4-fluoro-benzyl)-amine,
4-isopropoxy-6-(5-acetyl-2-chlorophenyl)-pteridin-2-yl]-(4-fluoro-benzyl)-amine,
4-isopropoxy-6-(4-acetyl-3-fluorophenyl)-pteridin-2-yl]-(4-fluoro-benzyl)-amine,
4-isopropoxy-6-(5-acetyl-2-fluorophenyl)-pteridin-2-yl]-(4-fluoro-benzyl)-amine,
4-isopropoxy-6-(3-aminophenyl)-pteridin-2-yl]-(4-fluoro-benzyl)-amine,
4-isopropoxy-6-(4-aminomethylphenyl)-pteridin-2-yl]-(4-fluoro-benzyl)-amine,
4-isopropoxy-6-(4-benzyloxy-2-fluorophenyl)-pteridin-2-yl]-(4-fluoro-benzyl)-amine,
4-isopropoxy-6-(4-benzyloxy-3-fluorophenyl)-pteridin-2-yl]-(4-fluoro-benzyl)-amine,
4-isopropoxy-6-(4-bromophenyl)-pteridin-2-yl]-(4-fluoro-benzyl)-amine,
4-isopropoxy-6-(3-bromophenyl)-pteridin-2-yl]-(4-fluoro-benzyl)-amine,
4-isopropoxy-6-(4-bromo-2,5-dimethylphenyl)-pteridin-2-yl]-(4-fluoro-benzyl)-amine,
4-isopropoxy-6-(2-bromo-5-fluorophenyl)-pteridin-2-yl]-(4-fluoro-benzyl)-amine,
4-isopropoxy-6-(2-bromo-6-fluorophenyl)-pteridin-2-yl]-(4-fluoro-benzyl)-amine,
4-isopropoxy-6-(2-carboxy-5-fluorophenyl)-pteridin-2-yl]-(4-fluoro-benzyl)-amine,
4-isopropoxy-6-(2-carboxyphenyl)-pteridin-2-yl]-(4-fluoro-benzyl)-amine,
4-isopropoxy-6-(4-carboxyphenyl)-pteridin-2-yl]-(4-fluoro-benzyl)-amine,
4-isopropoxy-6-(4-carboxy-2-chlorophenyl)-pteridin-2-yl]-(4-fluoro-benzyl)-amine,
4-isopropoxy-6-(5-carboxy-2-chlorophenyl)-pteridin-2-yl]-(4-fluoro-benzyl)-amine,
4-isopropoxy-6-(4-carboxy-3-chlorophenyl)-pteridin-2-yl]-(4-fluoro-benzyl)-amine,
4-isopropoxy-6-(3-carboxyphenyl)-pteridin-2-yl]-(4-fluoro-benzyl)-amine,
4-isopropoxy-6-(2-chloro-5-formylphenyl)-pteridin-2-yl]-(4-fluoro-benzyl)-amine,
4-isopropoxy-6-(2-chloro-5-hydroxyphenyl)-pteridin-2-yl]-(4-fluoro-benzyl)-amine,
4-isopropoxy-6-(3-chloro-4-fluorophenyl)-pteridin-2-yl]-(4-fluoro-benzyl)-amine,
4-isopropoxy-6-(2-chloro-4-fluorophenyl)-pteridin-2-yl]-(4-fluoro-benzyl)-amine,
4-isopropoxy-6-(4-chloro-2-fluorophenyl)-pteridin-2-yl]-(4-fluoro-benzyl)-amine,
4-isopropoxy-6-(3-chloro-5-methoxyphenyl)-pteridin-2-yl]-(4-fluoro-benzyl)-amine,
4-isopropoxy-6-(2-chloro-4-methylphenyl)-pteridin-2-yl]-(4-fluoro-benzyl)-amine,
4-isopropoxy-6-(2-chloro-5-methylphenyl)-pteridin-2-yl]-(4-fluoro-benzyl)-amine,
4-isopropoxy-6-(3-chloro-5-trifluoromethylphenyl)-pteridin-2-yl]-(4-fluoro-benzyl)-amine,
4-isopropoxy-6-(2-chloro-5-trifluoromethoxyphenyl)-pteridin-2-yl]-(4-fluoro-benzyl)-amine,
4-isopropoxy-6-(4-chloro-2-trifluoromethylphenyl)-pteridin-2-yl]-(4-fluor benzyl)-amine,
4-isopropoxy-6-(4-chlorophenyl)-pteridin-2-yl]-(4-fluoro-benzyl)-amine,
4-isopropoxy-6-(3-chlorophenyl)-pteridin-2-yl]-(4-fluoro-benzyl)-amine,
4-isopropoxy-6-(2-chlorophenyl)-pteridin-2-yl]-(4-fluoro-benzyl)-amine,
4-isopropoxy-6-(4-cyanophenyl)-pteridin-2-yl]-(4-fluoro-benzyl)-amine,
4-isopropoxy-6-(3-cyanophenyl)-pteridin-2-yl]-(4-fluoro-benzyl)-amine,
4-isopropoxy-6-(2-cyanophenyl)-pteridin-2-yl]-(4-fluoro-benzyl)-amine,
4-isopropoxy-6-(2,6-dichlorophenyl)-pteridin-2-yl]-(4-fluoro-benzyl)-amine,
4-isopropoxy-6-(3,4-dichlorophenyl)-pteridin-2-yl]-(4-fluoro-benzyl)-amine,
4-isopropoxy-6-(2,6-dichlorophenyl)-pteridin-2-yl]-(4-fluoro-benzyl)-amine,
4-isopropoxy-6-(2,4-dichlorophenyl)-pteridin-2-yl]-(4-fluoro-benzyl)-amine,
4-isopropoxy-6-(2,3-dichlorophenyl)-pteridin-2-yl]-(4-fluoro-benzyl)-amine,
4-isopropoxy-6-(3,5-dichlorophenyl)-pteridin-2-yl]-(4-fluoro-benzyl)-amine,
4-isopropoxy-6-(3,5-difluorophenyl)-pteridin-2-yl]-(4-fluoro-benzyl)-amine,
4-isopropoxy-6-(3,4-difluorophenyl)-pteridin-2-yl]-(4-fluoro-benzyl)-amine,
4-isopropoxy-6-(2,6-difluorophenyl)-pteridin-2-yl]-(4-fluoro-benzyl)-amine,
4-isopropoxy-6-(2,5-difluorophenyl)-pteridin-2-yl]-(4-fluoro-benzyl)-amine,
4-isopropoxy-6-(2,4-difluorophenyl)-pteridin-2-yl]-(4-fluoro-benzyl)-amine,
4-isopropoxy-6-(2,3-difluorophenyl)-pteridin-2-yl]-(4-fluoro-benzyl)-amine,
4-isopropoxy-6-(4-[N,N-dimethylamino]phenyl)-pteridin-2-yl]-(4-fluoro-benzyl)-amine,
4-isopropoxy-6-(2-[N,N-dimethylaminomethyl]phenyl)-pteridin-2-yl]-(4-fluoro-benzyl)-amine,
4-isopropoxy-6-(3,5-dimethylphenyl)-pteridin-2-yl]-(4-fluorobenzyl)-amine,
4-isopropoxy-6-(3,4-dimethylphenyl)-pteridin-2-yl]-(4-fluorobenzyl)-amine,
4-isopropoxy-6-(2,6-dimethylphenyl)-pteridin-2-yl]-(4-fluorobenzyl)-amine,
4-isopropoxy-6-(2,6-dimethoxyphenyl)-pteridin-2-yl]-(4-fluorobenzyl)-amine,
4-isopropoxy-6-(2,5-dimethoxyphenyl)-pteridin-2-yl]-(4-fluorobenzyl)-amine,
4-isopropoxy-6-(2,4-dimethoxyphenyl)-pteridin-2-yl]-(4-fluorobenzyl)-amine,
4-isopropoxy-6-(4-ethoxyphenyl)-pteridin-2-yl]-(4-fluorobenzyl)-amine,
4-isopropoxy-6-(2-ethoxyphenyl)-pteridin-2-yl]-(4-fluorobenzyl)-amine,
4-isopropoxy-6-(4-ethoxycarbonylphenyl)-pteridin-2-yl]-(4-fluorobenzyl)-amine,
4-isopropoxy-6-(3-ethoxycarbonylphenyl)-pteridin-2-yl]-(4-fluorobenzyl)-amine,
4-isopropoxy-6-(4-ethylphenyl)-pteridin-2-yl]-(4-fluorobenzyl)-amine,
4-isopropoxy-6-(4-fluorophenyl)-pteridin-2-yl]-(4-fluorobenzyl)-amine,
4-isopropoxy-6-(3-fluorophenyl)-pteridin-2-yl]-(4-fluorobenzyl)-amine,
4-isopropoxy-6-(2-fluorophenyl)-pteridin-2-yl]-(4-fluorobenzyl)-amine,
4-isopropoxy-6-(3-fluoro-4-formylphenyl)-pteridin-2-yl]-(4-fluorobenzyl)-amine,
4-isopropoxy-6-(4-fluoro-3-formylphenyl)-pteridin-2-yl]-(4-fluorobenzyl)-amine,
4-isopropoxy-6-(4-fluoro-2-methylphenyl)-pteridin-2-yl]-(4-fluorobenzyl)-amine,
4-isopropoxy-6-(2-fluoro-5-methylphenyl)-pteridin-2-yl]-(4-fluorobenzyl)-amine,
4-isopropoxy-6-(2-fluoro-5-methoxyphenyl)-pteridin-2-yl]-(4-fluorobenzyl)-amine,
4-isopropoxy-6-(5-fluoro-2-methoxycarbonylphenyl)-pteridin-2-yl]-(4-fluorobenzyl)-amine,
4-isopropoxy-6-(2-formyl-5-methoxyphenyl)-pteridin-2-yl]-(4-fluorobenzyl)-amine,
4-isopropoxy-6-(5-formyl-2-methoxyphenyl)-pteridin-2-yl]-(4-fluorobenzyl)-amine,
4-isopropoxy-6-(2-formyl-5-methylphenyl)-pteridin-2-yl]-(4-fluorobenzyl)-amine,
4-isopropoxy-6-(2-formylphenyl)-pteridin-2-yl]-(4-fluorobenzyl)-amine,
4-isopropoxy-6-(3-formylphenyl)-pteridin-2-yl]-(4-fluorobenzyl)-amine,
4-isopropoxy-6-(4-formylphenyl)-pteridin-2-yl]-(4-fluorobenzyl)-amine,
4-isopropoxy-6-(3-hydroxy-4-methoxycarbonylphenyl)-pteridin-2-yl]-(4-fluorobenzyl)-amine,
4-isopropoxy-6-(3-hydroxymethylphenyl)-pteridin-2-yl]-(4-fluorobenzyl)-amine,
4-isopropoxy-6-(4-hydroxymethylphenyl)-pteridin-2-yl]-(4-fluorobenzyl)-amine,
4-isopropoxy-6-(4-hydroxyphenyl)-pteridin-2-yl]-(4-fluorobenzyl)-amine,
4-isopropoxy-6-(3-hydroxyphenyl)-pteridin-2-yl]-(4-fluorobenzyl)-amine,
4-isopropoxy-6-(4-iodophenyl)-pteridin-2-yl]-(4-fluorobenzyl)-amine,
4-isopropoxy-6-(3-iodophenyl)-pteridin-2-yl]-(4-fluorobenzyl)-amine,
4-isopropoxy-6-(4-isopropoxycarbonylphenyl)-pteridin-2-yl]-(4-fluorobenzyl)-amine,
4-isopropoxy-6-(3-isopropoxycarbonylphenyl)-pteridin-2-yl]-(4-fluorobenzyl)-amine,
4-isopropoxy-6-(4-methanesulfonylphenyl)-pteridin-2-yl]-(4-fluorobenzyl)-amine,
4-isopropoxy-6-(4-methoxy-2-formylphenyl)-pteridin-2-yl]-(4-fluorobenzyl)-amine,
4-isopropoxy-6-(4-methoxycarbonylphenyl)-pteridin-2-yl]-(4-fluorobenzyl)-amine,
4-isopropoxy-6-(3-methoxycarbonylphenyl)-pteridin-2-yl]-(4-fluorobenzyl)-amine,
4-isopropoxy-6-(4-methoxyphenyl)-pteridin-2-yl]-(4-fluorobenzyl)-amine,
4-isopropoxy-6-(3-methoxyphenyl)-pteridin-2-yl]-(4-fluorobenzyl)-amine,
4-isopropoxy-6-(2-methoxyphenyl)-pteridin-2-yl]-(4-fluorobenzyl)-amine,
4-isopropoxy-6-(3,4-methylenedioxyphenyl)-pteridin-2-yl]-(4-fluorobenzyl)-amine,
4-isopropoxy-6-(4-methylphenyl)-pteridin-2-yl]-(4-fluorobenzyl)-amine,
4-isopropoxy-6-(2-methylphenyl)-pteridin-2-yl]-(4-fluorobenzyl)-amine,
4-isopropoxy-6-(4-(methylthio)phenyl)-pteridin-2-yl]-(4-fluorobenzyl)-amine,
4-isopropoxy-6-(3-(methylthio)phenyl)-pteridin-2-yl]-(4-fluorobenzyl)-amine,
4-isopropoxy-6-(4-morpholinophenyl)-pteridin-2-yl]-(4-fluorobenzyl)-amine,
4-isopropoxy-6-(3-nitrophenyl)-pteridin-2-yl]-(4-fluorobenzyl)-amine,
4-isopropoxy-6-(4-phenoxyphenyl)-pteridin-2-yl]-(4-fluorobenzyl)-amine,
4-isopropoxy-6-(4-(tert-butoxycarbonyl)phenyl)-pteridin-2-yl]-(4-fluoro-benzyl)-amine,
4-isopropoxy-6-(3-(tert-butoxycarbonyl)phenyl)-pteridin-2-yl]-(4-fluoro-benzyl)-amine,
4-isopropoxy-6-(2-(tert-butoxycarbonyl)phenyl)-pteridin-2-yl]-(4-fluoro-benzyl)-amine,
4-isopropoxy-6-(4-(tert-butoxycarbonylamino)-3-methoxyphenyl)-pteridin-2-yl]-(4-fluorobenzyl)-amine,
4-isopropoxy-6-(4-(tert-butylphenyl)-pteridin-2-yl]-(4-fluorobenzyl)-amine,
4-isopropoxy-6-(4-(2-thienyl)phenyl)-pteridin-2-yl]-(4-fluorobenzyl)-amine,
4-isopropoxy-6-(4-trifluoromethoxyphenyl)-pteridin-2-yl]-(4-fluorobenzyl)-amine,
4-isopropoxy-6-(2,4,6-trimethylphenyl)-pteridin-2-yl]-(4-fluorobenzyl)-amine,
4-isopropoxy-6-(3,4,6-trifluorophenyl)-pteridin-2-yl]-(4-fluorobenzyl)-amine,
4-isopropoxy-6-(3-trifluoromethoxyphenyl)-pteridin-2-yl]-(4-fluorobenzyl)-amine,
4-isopropoxy-6-(3-trifluoromethylphenyl)-pteridin-2-yl]-(4-fluorobenzyl)-amine,
4-isopropoxy-6-(2-trifluoromethylphenyl)-pteridin-2-yl]-(4-fluorobenzyl)-amine,
4-isopropoxy-6-(3,4,5-trimethoxyphenyl)-pteridin-2-yl]-(4-fluorobenzyl)-amine,
4-isopropoxy-6-(4-vinylphenyl)-pteridin-2-yl]-(4-fluorobenzyl)-amine,
4-isopropoxy-6-(2-acetamidopyridin-5-yl)-pteridin-2-yl]-(4-fluorobenzyl)-amine,
4-isopropoxy-6-(2-benzothienyl)-pteridin-2-yl]-(4-fluorobenzyl)-amine,
4-isopropoxy-6-(1-benzothiophen-3-yl)-pteridin-2-yl]-(4-fluorobenzyl)-amine,
4-isopropoxy-6-(1-benzothiophen-2-yl)-pteridin-2-yl]-(4-fluorobenzyl)-amine,
4-isopropoxy-6-(2-bromo-3-chloropyridin-4-yl)-pteridin-2-yl]-(4-fluoro-benzyl)-amine,
4-isopropoxy-6-(2-bromo-3-methylpyridin-5-yl)-pteridin-2-yl]-(4-fluoro-benzyl)-amine,
4-isopropoxy-6-(2-bromopyridin-5-yl)-pteridin-2-yl]-(4-fluorobenzyl)-amine,
4-isopropoxy-6-(2-chloro-3-methylpyridin-5-yl)-pteridin-2-yl]-(4-fluoro-benzyl)-amine,
4-isopropoxy-6-(2-chloro-6-isopropylpyridin-3-yl)-pteridin-2-yl]-(4-fluoro-benzyl)-amine,
4-isopropoxy-6-(5-chlorothien-2-yl)-pteridin-2-yl]-(4-fluoro-benzyl)-amine,
4-isopropoxy-6-(5-bromothien-2-yl)-pteridin-2-yl]-(4-fluoro-benzyl)-amine,
4-isopropoxy-6-(2-chloro-3-fluoropyridin-4-yl)-pteridin-2-yl]-(4-fluoro-benzyl)-amine,
4-isopropoxy-6-(2,5-dibromopyridin-3-yl)-pteridin-2-yl]-(4-fluoro-benzyl)-amine,
4-isopropoxy-6-(2,6-dichloropyridin-3-yl)-pteridin-2-yl]-(4-fluoro-benzyl)-amine,
4-isopropoxy-6-(2,4-dimethoxypyrimidin-5-yl)-pteridin-2-yl]-(4-fluoro-benzyl)-amine,
4-isopropoxy-6-(3,5-dimethylisoxazol-4-yl)-pteridin-2-yl]-(4-fluoro-benzyl)-amine,
4-isopropoxy-6-(2,4-dioxo-1,2,3,4-tetrahydropyrimidin-5-yl)-pteridin-2-yl]-(4-fluoro-benzyl)-amine,
4-isopropoxy-6-(2-ethoxypyridin-3-yl)-pteridin-2-yl]-(4-fluorobenzyl)-amine,
4-isopropoxy-6-(2-fluoro-3-methylpyridin-5-yl)-pteridin-2-yl]-(4-fluorobenzyl)-amine,
4-isopropoxy-6-(2-fluoropyridin-3-yl)-pteridin-2-yl]-(4-fluorobenzyl)-amine,
4-isopropoxy-6-(2-fluoropyridin-5-yl)-pteridin-2-yl]-(4-fluorobenzyl)-amine,
4-isopropoxy-6-(5-formyl-2-furyl)-pteridin-2-yl]-(4-fluorobenzyl)-amine,
4-isopropoxy-6-(5-formyl-thiophen-2-yl)-pteridin-2-yl]-(4-fluorobenzyl)-amine,
4-isopropoxy-6-(furan-3-yl)-pteridin-2-yl]-(4-fluorobenzyl)-amine,
4-isopropoxy-6-(furan-2-yl)-pteridin-2-yl]-(4-fluorobenzyl)-amine,
4-isopropoxy-6-(indol-5-yl)-pteridin-2-yl]-(4-fluorobenzyl)-amine,
4-isopropoxy-6-(isoquinolin-4-yl)-pteridin-2-yl]-(4-fluorobenzyl)-amine,
4-isopropoxy-6-(5-methylfuran-2-yl)-pteridin-2-yl]-(4-fluorobenzyl)-amine,
4-isopropoxy-6-(methoxypyrimidin-5-yl)-pteridin-2-yl]-(4-fluorobenzyl)-amine,
4-isopropoxy-6-(5-methyl-1-benzothiophen-2-yl)-pteridin-2-yl]-(4-fluorobenzyl)-amine,
4-isopropoxy-6-(5-methyl-3-phenyl-4-isoxazolyl)-pteridin-2-yl]-(4-fluorobenzyl)-amine,
4-isopropoxy-6-(3-methylpyridin-2-yl)-pteridin-2-yl]-(4-fluorobenzyl)-amine,
4-isopropoxy-6-(5-methylpyridin-2-yl)-pteridin-2-yl]-(4-fluorobenzyl)-amine,
4-isopropoxy-6-(5-methylpyridin-3-yl)-pteridin-2-yl]-(4-fluorobenzyl)-amine,
4-isopropoxy-6-(2-methoxypyridin-3-yl)-pteridin-2-yl]-(4-fluorobenzyl)-amine,
4-isopropoxy-6-(2-methoxypyridin-5-yl)-pteridin-2-yl]-(4-fluorobenzyl)-amine,
4-isopropoxy-6-(pyridin-4-yl)-pteridin-2-yl]-(4-fluorobenzyl)-amine,
4-isopropoxy-6-(pyridin-3-yl)-pteridin-2-yl]-(4-fluorobenzyl)-amine,
4-isopropoxy-6-(pyrimidin-5-yl)-pteridin-2-yl]-(4-fluorobenzyl)-amine,
4-isopropoxy-6-(quinolin-3-yl)-pteridin-2-yl]-(4-fluorobenzyl)-amine,
4-isopropoxy-6-(quinolin-8-yl)-pteridin-2-yl]-(4-fluorobenzyl)-amine, and
4-isopropoxy-6-(thien-2-yl)-pteridin-2-yl]-(4-fluorobenzyl)-amine.

### Examples 700 to 744

### Synthesis of 4-ethoxy-6-(4-fluorophenyl)-pteridin-2-yl]-(4-fluoro-benzyl)-amine Analogues

The procedure of the first step of example C is repeated while replacing 4-fluorobenzaldehyde with an alternative optionally substituted benzaldehyde. In this way, the following analogues are obtained in similar yields:
4-ethoxy-6-(4-fluorophenyl)-pteridin-2-yl]-benzylamine,
4-ethoxy-6-(4-fluorophenyl)-pteridin-2-yl]-(4-methylbenzyl)-amine,
4-ethoxy-6-(4-fluorophenyl)-pteridin-2-yl]-(3-methylbenzyl)-amine,
4-ethoxy-6-(4-fluorophenyl)-pteridin-2-yl]-(2-methylbenzyl)-amine,
4-ethoxy-6-(4-fluorophenyl)-pteridin-2-yl]-(4-methoxybenzyl)-amine,
4-ethoxy-6-(4-fluorophenyl)-pteridin-2-yl]-(3-methoxybenzyl)-amine,
4-ethoxy-6-(4-fluorophenyl)-pteridin-2-yl]-(2-methoxybenzyl)-amine,
4-ethoxy-6-(4-fluorophenyl)-pteridin-2-yl]-(2,5-dihydroxybenzyl)-amine,
4-ethoxy-6-(4-fluorophenyl)-pteridin-2-yl]-(4-propoxybenzyl)-amine,
4-ethoxy-6-(4-fluorophenyl)-pteridin-2-yl]-(4-phenoxybenzyl)-amine,
4-ethoxy-6-(4-fluorophenyl)-pteridin-2-yl]-(3-[3,4-dichlorophenoxy]-benzyl)-amine,
4-ethoxy-6-(4-fluorophenyl)-pteridin-2-yl]-(3-[3,5-dichlorophenoxy]-benzyl)-amine,
4-ethoxy-6-(4-fluorophenyl)-pteridin-2-yl]-(2-bromobenzyl)-amine,
4-ethoxy-6-(4-fluorophenyl)-pteridin-2-yl]-(3-bromobenzyl)-amine,
4-ethoxy-6-(4-fluorophenyl)-pteridin-2-yl]-(4-bromobenzyl)-amine,
4-ethoxy-6-(4-fluorophenyl)-pteridin-2-yl]-(2-chlorobenzyl)-amine,
4-ethoxy-6-(4-fluorophenyl)-pteridin-2-yl]-(3-chlorobenzyl)-amine,
4-ethoxy-6-(4-fluorophenyl)-pteridin-2-yl]-(4-chlorobenzyl)-amine,
4-ethoxy-6-(4-fluorophenyl)-pteridin-2-yl]-(2-fluorobenzyl)-amine,
4-ethoxy-6-(4-fluorophenyl)-pteridin-2-yl]-(3-fluorobenzyl)-amine,
4-ethoxy-6-(4-fluorophenyl)-pteridin-2-yl]-(2,3-dichlorobenzyl)-amine,
4-ethoxy-6-(4-fluorophenyl)-pteridin-2-yl]-(2,4-dichlorobenzyl)-amine,
4-ethoxy-6-(4-fluorophenyl)-pteridin-2-yl]-(2,6-dichlorobenzyl)-amine,
4-ethoxy-6-(4-fluorophenyl)-pteridin-2-yl]-(3,4-dichlorobenzyl)-amine,
4-ethoxy-6-(4-fluorophenyl)-pteridin-2-yl]-(3,5-dichlorobenzyl)-amine,
4-ethoxy-6-(4-fluorophenyl)-pteridin-2-yl]-(2,3-difluorobenzyl)-amine,
4-ethoxy-6-(4-fluorophenyl)-pteridin-2-yl]-(2,4-difluorobenzyl)-amine,
4-ethoxy-6-(4-fluorophenyl)-pteridin-2-yl]-(2,5-difluorobenzyl)-amine,
4-ethoxy-6-(4-fluorophenyl)-pteridin-2-yl]-(2,6-difluorobenzyl)-amine,
4-ethoxy-6-(4-fluorophenyl)-pteridin-2-yl]-(3,4-difluorobenzyl)-amine,
4-ethoxy-6-(4-fluorophenyl)-pteridin-2-yl]-(3,5-difluorobenzyl)-amine,
4-ethoxy-6-(4-fluorophenyl)-pteridin-2-yl]-(2,3,4-trifluorobenzyl)-amine,
4-ethoxy-6-(4-fluorophenyl)-pteridin-2-yl]-(2-trifluoromethylbenzyl)-amine,
4-ethoxy-6-(4-fluorophenyl)-pteridin-2-yl]-(3-trifluoromethylbenzyl)-amine,
4-ethoxy-6-(4-fluorophenyl)-pteridin-2-yl]-(4-trifluoromethylbenzyl)-amine,
4-ethoxy-6-(4-fluorophenyl)-pteridin-2-yl]-(3-trifluoromethoxybenzyl)-amine,
4-ethoxy-6-(4-fluorophenyl)-pteridin-2-yl]-(5-trifluoromethoxybenzyl)-amine,
4-ethoxy-6-(4-fluorophenyl)-pteridin-2-yl]-(2-nitrobenzyl)-amine,
4-ethoxy-6-(4-fluorophenyl)-pteridin-2-yl]-(3-nitrobenzyl)-amine,
4-ethoxy-6-(4-fluorophenyl)-pteridin-2-yl]-(4-nitrobenzyl)-amine,
4-ethoxy-6-(4-fluorophenyl)-pteridin-2-yl]-(3-cyanobenzyl)-amine,
4-ethoxy-6-(4-fluorophenyl)-pteridin-2-yl]-(4-cyanobenzyl)-amine,
4-ethoxy-6-(4-fluorophenyl)-pteridin-2-yl]-(4-dimethylaminobenzyl)-amine,
4-ethoxy-6-(4-fluorophenyl)-pteridin-2-yl]-(4-diethylaminobenzyl)-amine, and
4-ethoxy-6-(4-fluorophenyl)-pteridin-2-yl]-(3,4,5-trimethoxybenzyl)-amine.

### Examples 750 to 794

### Synthesis of 4-isopropoxy-6-(4-fluorophenyl)-pteridin-2-yl]-(4-fluorobenzyl)-amine Analogues

The procedure of example C is repeated, except that the first step is carried out starting from 6-chloro-4-ethoxy-pteridin-2-ylamine and that 4-fluorobenzaldehyde is replaced in the first step with an alternative optionally substituted benzaldehyde. In this way, the following analogues are obtained in similar yields:
4-isopropoxy-6-(4-fluorophenyl)-pteridin-2-yl]-benzylamine,
4-isopropoxy-6-(4-fluorophenyl)-pteridin-2-yl]-(4-methylbenzyl)-amine,
4-isopropoxy-6-(4-fluorophenyl)-pteridin-2-yl]-(3-methylbenzyl)-amine,
4-isopropoxy-6-(4-fluorophenyl)-pteridin-2-yl]-(2-methylbenzyl)-amine,
4-isopropoxy-6-(4-fluorophenyl)-pteridin-2-yl]-(4-methoxybenzyl)-amine,
4-isopropoxy-6-(4-fluorophenyl)-pteridin-2-yl]-(3-methoxybenzyl)-amine,
4-isopropoxy-6-(4-fluorophenyl)-pteridin-2-yl]-(2-methoxybenzyl)-amine,
4-isopropoxy-6-(4-fluorophenyl)-pteridin-2-yl]-(2,5-dihydroxybenzyl)-amine,
4-isopropoxy-6-(4-fluorophenyl)-pteridin-2-yl]-(4-propoxybenzyl)-amine,
4-isopropoxy-6-(4-fluorophenyl)-pteridin-2-yl]-(4-phenoxybenzyl)-amine,
4-isopropoxy-6-(4-fluorophenyl)-pteridin-2-yl]-(3-[3,4-dichlorophenoxy]-benzyl)-amine,
4-isopropoxy-6-(4-fluorophenyl)-pteridin-2-yl]-(3-[3,5-dichlorophenoxy]-benzyl)-amine,
4-isopropoxy-6-(4-fluorophenyl)-pteridin-2-yl]-(2-bromobenzyl)-amine,
4-isopropoxy-6-(4-fluorophenyl)-pteridin-2-yl]-(3-bromobenzyl)-amine,
4-isopropoxy-6-(4-fluorophenyl)-pteridin-2-yl]-(4-bromobenzyl)-amine,
4-isopropoxy-6-(4-fluorophenyl)-pteridin-2-yl]-(2-chlorobenzyl)-amine,
4-isopropoxy-6-(4-fluorophenyl)-pteridin-2-yl]-(3-chlorobenzyl)-amine,
4-isopropoxy-6-(4-fluorophenyl)-pteridin-2-yl]-(4-chlorobenzyl)-amine,
4-isopropoxy-6-(4-fluorophenyl)-pteridin-2-yl]-(2-fluorobenzyl)-amine,
4-isopropoxy-6-(4-fluorophenyl)-pteridin-2-yl]-(3-fluorobenzyl)-amine,
4-isopropoxy-6-(4-fluorophenyl)-pteridin-2-yl]-(2,3-dichlorobenzyl)-amine,
4-isopropoxy-6-(4-fluorophenyl)-pteridin-2-yl]-(2,4-dichlorobenzyl)-amine,
4-isopropoxy-6-(4-fluorophenyl)-pteridin-2-yl]-(2,6-dichlorobenzyl)-amine,
4-isopropoxy-6-(4-fluorophenyl)-pteridin-2-yl]-(3,4-dichlorobenzyl)-amine,
4-isopropoxy-6-(4-fluorophenyl)-pteridin-2-yl]-(3,5-dichlorobenzyl)-amine,
4-isopropoxy-6-(4-fluorophenyl)-pteridin-2-yl]-(2,3-difluorobenzyl)-amine,
4-isopropoxy-6-(4-fluorophenyl)-pteridin-2-yl]-(2,4-difluorobenzyl)-amine,
4-isopropoxy-6-(4-fluorophenyl)-pteridin-2-yl]-(2,5-difluorobenzyl)-amine,
4-isopropoxy-6-(4-fluorophenyl)-pteridin-2-yl]-(2,6-difluorobenzyl)-amine,
4-isopropoxy-6-(4-fluorophenyl)-pteridin-2-yl]-(3,4-difluorobenzyl)-amine,
4-isopropoxy-6-(4-fluorophenyl)-pteridin-2-yl]-(3,5-difluorobenzyl)-amine,
4-isopropoxy-6-(4-fluorophenyl)-pteridin-2-yl]-(2,3,4-trifluorobenzyl)-amine,
4-isopropoxy-6-(4-fluorophenyl)-pteridin-2-yl]-(2-trifluoromethylbenzyl)-amine,
4-isopropoxy-6-(4-fluorophenyl)-pteridin-2-yl]-(3-trifluoromethylbenzyl)-amine,
4-isopropoxy-6-(4-fluorophenyl)-pteridin-2-yl]-(4-trifluoromethylbenzyl)-amine,
4-isopropoxy-6-(4-fluorophenyl)-pteridin-2-yl]-(3-trifluoromethoxybenzyl)-amine,
4-isopropoxy-6-(4-fluorophenyl)-pteridin-2-yl]-(5-trifluoromethoxybenzyl)-amine,
4-isopropoxy-6-(4-fluorophenyl)-pteridin-2-yl]-(2-nitrobenzyl)-amine,
4-isopropoxy-6-(4-fluorophenyl)-pteridin-2-yl]-(3-nitrobenzyl)-amine,
4-isopropoxy-6-(4-fluorophenyl)-pteridin-2-yl]-(4-nitrobenzyl)-amine,
4-isopropoxy-6-(4-fluorophenyl)-pteridin-2-yl]-(3-cyanobenzyl)-amine,
4-isopropoxy-6-(4-fluorophenyl)-pteridin-2-yl]-(4-cyanobenzyl)-amine,
4-isopropoxy-6-(4-fluorophenyl)-pteridin-2-yl]-(4-dimethylaminobenzyl)-amine,
4-isopropoxy-6-(4-fluorophenyl)-pteridin-2-yl]-(4-diethylaminobenzyl)-amine, and
4-isopropoxy-6-(4-fluorophenyl)-pteridin-2-yl]-(3,4,5-trimethoxybenzyl)-amine.

### Examples 800 to 831

### Synthesis of 4-{[6-(4-fluorophenyl)-4-(2,2,2-trifluoro-ethylamino)-pteridin-2-ylamino]-methyl}-benzenesulfonamide Analogues

The procedure of example D is repeated, except that in the last step 4-aminomethyl-benzenesulfonamide hydrochloride is replaced with another optionally substituted benzylamine. In this way, the following analogues are obtained in similar yields:
2-{[6-(4-fluorophenyl)-4-(2,2,2-trifluoroethylamino)-pteridin-2-ylamino]-methyl}-chlorobenzene,
4-{[6-(4-fluorophenyl)-4-(2,2,2-trifluoroethylamino)-pteridin-2-ylamino]-methyl}-chlorobenzene,
4-{[6-(4-fluorophenyl)-4-(2,2,2-trifluoroethylamino)-pteridin-2-ylamino]-methyl}-methylbenzene,
4-{[6-(4-fluorophenyl)-4-(2,2,2-trifluoroethylamino)-pteridin-2-ylamino]-methyl}-methoxybenzene,
3-{[6-(4-fluorophenyl)-4-(2,2,2-trifluoroethylamino)-pteridin-2-ylamino]-methyl}-methylbenzene,
2-{[6-(4-fluorophenyl)-4-(2,2,2-trifluoroethylamino)-pteridin-2-ylamino]-methyl}-methylbenzene,
2-{[6-(4-fluorophenyl)-4-(2,2,2-trifluoroethylamino)-pteridin-2-ylamino]-methyl}-methoxybenzene,
3-{[6-(4-fluorophenyl)-4-(2,2,2-trifluoroethylamino)-pteridin-2-ylamino]-methyl}-methoxybenzene,
3-{[6-(4-fluorophenyl)-4-(2,2,2-trifluoroethylamino)-pteridin-2-ylamino]-methyl}-fluorobenzene,
2-{[6-(4-fluorophenyl)-4-(2,2,2-trifluoroethylamino)-pteridin-2-ylamino]-methyl}-fluorobenzene,
4-{[6-(4-fluorophenyl)-4-(2,2,2-trifluoroethylamino)-pteridin-2-ylamino]-methyl}-fluorobenzene,
3-{[6-(4-fluorophenyl)-4-(2,2,2-trifluoroethylamino)-pteridin-2-ylamino]-methyl}-chlorobenzene,
4-{[6-(4-fluorophenyl)-4-(2,2,2-trifluoroethylamino)-pteridin-2-ylamino]-methyl}-trifluoromethoxybenzene,
2-{[6-(4-fluorophenyl)-4-(2,2,2-trifluoroethylamino)-pteridin-2-ylamino]-methyl}-trifluoromethoxybenzene,
4-{[6-(4-fluorophenyl)-4-(2,2,2-trifluoroethylamino)-pteridin-2-ylamino]-methyl}-trifluoromethylbenzene,
2-{[6-(4-fluorophenyl)-4-(2,2,2-trifluoroethylamino)-pteridin-2-ylamino]-methyl}-trifluoromethylbenzene,
3-{[6-(4-fluorophenyl)-4-(2,2,2-trifluoroethylamino)-pteridin-2-ylamino]-methyl}-trifluoromethylbenzene,
2-{[6-(4-fluorophenyl)-4-(2,2,2-trifluoroethylamino)-pteridin-2-ylamino]-methyl}-bromobenzene,
4-{[6-(4-fluorophenyl)-4-(2,2,2-trifluoroethylamino)-pteridin-2-ylamino]-methyl}-bromobenzene,
3-{[6-(4-fluorophenyl)-4-(2,2,2-trifluoroethylamino)-pteridin-2-ylamino]-methyl}-bromobenzene,
3-{[6-(4-fluorophenyl)-4-(2,2,2-trifluoroethylamino)-pteridin-2-ylamino]-methyl}-iodobenzene,
2-{[6-(4-fluorophenyl)-4-(2,2,2-trifluoroethylamino)-pteridin-2-ylamino]-methyl}-hydroxybenzene,
4-{[6-(4-fluorophenyl)-4-(2,2,2-trifluoroethylamino)-pteridin-2-ylamino]-methyl}-thien-2-ylbenzene,
4-{[6-(4-fluorophenyl)-4-(2,2,2-trifluoroethylamino)-pteridin-2-ylamino]-methyl}-morpholinobenzene,
2-{[6-(4-fluorophenyl)-4-(2,2,2-trifluoroethylamino)-pteridin-2-ylamino]-methyl}-morpholinobenzene,
4-{[6-(4-fluorophenyl)-4-(2,2,2-trifluoroethylamino)-pteridin-2-ylamino]-methyl}-morpholinomethylbenzene,
4-{[6-(4-fluorophenyl)-4-(2,2,2-trifluoroethylamino)-pteridin-2-ylamino]-methyl}-(4-methylpiperazino)benzene,
2-{[6-(4-fluorophenyl)-4-(2,2,2-trifluoroethylamino)-pteridin-2-ylamino]-methyl}-methylthiobenzene,
3-{[6-(4-fluorophenyl)-4-(2,2,2-trifluoroethylamino)-pteridin-2-ylamino]-methyl}-phenoxymethylbenzene,
4-{[6-(4-fluorophenyl)-4-(2,2,2-trifluoroethylamino)-pteridin-2-ylamino]-methyl}-(pyrrolidin-1-ylmethyl)benzene,
2-{[6-(4-fluorophenyl)-4-(2,2,2-trifluoroethylamino)-pteridin-2-ylamino]-methyl}-(pyrrolidin-1-ylmethyl)benzene, and
3-{[6-(4-fluorophenyl)-4-(2,2,2-trifluoroethylamino)-pteridin-2-ylamino]-methyl}-(pyrimidin-2-yl)benzene.

1-33. (canceled)

34. A method of treatment or prevention of an infection due to a virus from the Flaviridae family, consisting essentially of administering to a patient in need thereof a therapeutically effective amount of a pteridine derivative having the structural formula (I): 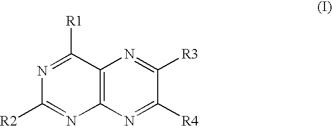
wherein:
R₁ is selected from the group consisting of hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl;
R₂ is selected from the group consisting of amino, hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl,
R₃ is selected from the group consisting of unsubstituted, monosubstituted and disubstituted aryl groups wherein the optional substituent(s) may be, but are not limited to, halogen, C₁₋₄ alkoxy, C₁₋₄ alkyl, aryl groups bonded to the pteridine ring via a saturated or unsaturated aliphatic spacer which may be halogenated or hydroxylated, and aliphatic substituents which may contain an ether, hydroxy, amino or C₁₋₄ alkoxy group; and
R₄ is selected from the group consisting of hydrogen, alkyl, alkoxy, substituted and non-substituted aryl groups,
and/or a pharmaceutically acceptable addition salt thereof, and/or a stereoisomer thereof, and/or a mono- or a di-N-oxide thereof, and/or a solvate thereof, and/or a dihydro- or tetrahydropteridine derivative thereof, and/or a pro-drug thereof, with the proviso that said pteridine derivative is not 2-amino-4-ethoxy-6-(4-fluorophenyl)-pteridine or 2-amino-4-isopropoxy-6-(4-fluorophenyl)-pteridine.

35. A method according to claim 34, wherein said administration is oral administration.

36. A method according to claim 34, wherein said virus is HCV.

37. A method according to claim 34, wherein said therapeutically effective amount is from 0.1 mg to 5 mg per day per kg bodyweight of said patient.

38. A method of treatment or prevention of an infection due to a virus from the Flaviridae family, consisting essentially of administering to a patient in need thereof a therapeutically effective amount of a pteridine derivative having the structural formula (II) 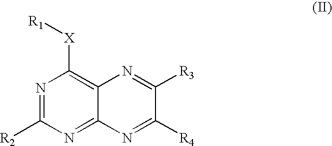
wherein X represents an oxygen atom or a group with the formula S(O)ₘ wherein m is an integer from 0 to 2, or a group with the formula NZ and wherein:
R₁ is a group selected from the group consisting of C₁₋₇ alkyl, C₂₋₇ alkenyl, C₂₋₇ alkynyl, C₃₋₁₀ cycloalkyl, C₃₋₁₀ cycloalkenyl, aryl, alkylaryl, arylalkyl, heterocyclic, heterocyclic-substituted alkyl and alkyl-substituted heterocyclic, each of said groups being optionally substituted with one or more substituents selected from the group consisting of halogen, C₁₋₄ alkyl and C₁₋₄ alkoxy; or R₁ is a carboxyalkyl, carboxyaryl, thiocarboxyaryl or thiocarboxyalkyl group;
Z is a group independently defined as R₁ or Z is hydrogen or the group NZ together with R₁ is either hydroxylamino or an optionally substituted heterocyclic group containing at least one nitrogen atom;
R₂ is selected from the group consisting of amino; acylamino; thioacylamino; carbamoyl; thiocarbamoyl, ureido; thio-ureido, sulfonamido; hydroxylamino; alkoxyamino; thioalkylamino; mercaptoamino, hydrazino; alkylhydrazino; phenylhydrazino; optionally substituted heterocyclic radicals; C₃₋₇ alkylamino; arylamino; arylalkylamino; cycloalkylamino; alkenylamino; cycloalkenylamino; heterocyclic amino; hydroxyalkylamino; mercaptoalkylamino; C₁₋₇ alkoxy; C₃₋₁₀ cycloalkoxy; thio C₁₋₇ alkyl; arylsulfoxide; arylsulfone; heterocyclic sulfoxide; heterocyclic sulfone; thio C₃₋₁₀ cycloalkyl; aryloxy; arylthio; arylalkyloxy; arylalkylthio; oxyheterocyclic and thioheterocyclic radicals;
R₄ is an atom or a group selected from the group consisting of hydrogen; halogen; C₁₋₇ alkyl; C₂₋₇ alkenyl; C₂₋₇ alkynyl; halo C₁₋₇ alkyl; carboxy C₁₋₇ alkyl; carboxyaryl; C₁₋₇ alkoxy; C₃₋₁₀ cycloalkoxy; aryloxy; arylalkyloxy; oxyheterocyclic; heterocyclic-substituted alkyloxy; thio C₁₋₇ alkyl; thio C₃₋₁₀ cycloalkyl; thioaryl; thioheterocyclic; arylalkylthio; heterocyclic-substituted alkylthio; hydroxylamino; mercapto-amino; acylamino; thio-acylamino; alkoxyamino; thioalkylamino; acetal; thio-acetal; carboxylic acid; carboxylic acid esters, thioesters, halides, anhydrides, amides and thioamides; thiocarboxylic acid; thiocarboxylic acid esters, thioesters, halides, anhydrides, amides and thioamides; hydroxyl; sulfhydryl; nitro; cyano; carbamoyl; thiocarbamoyl, ureido; thio-ureido; alkylamino; cycloalkylamino; alkenylamino; cycloalkenyl-amino; alkynylamino; arylamino; arylalkylamino; hydroxyalkylamino; mercaptoalkylamino; heterocyclic amino; heterocyclic-substituted alkylamino; oximino; alkyloximino; hydrazino; alkylhydrazino; phenylhydrazino; cysteinyl acid, esters, thioesters, halides, anhydrides, amides and thioamides thereof; phenyl substituted with one or more substituents selected from the group consisting of C₁₋₇ alkyl, C₂₋₇ alkenyl, C₂₋₇ alkynyl, halo C₁₋₇ alkyl, nitro, hydroxyl, sulfhydryl, amino, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, carbamoyl, thiocarbamoyl, ureido, thio-ureido, sulfonamido, hydroxylamino, alkoxyamino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cycloalkenylamino, alkynyl-amino, arylamino, arylalkylamino, hydroxyalkylamino, mercaptoalkylamino, heterocyclic amino, hydrazino, alkylhydrazino and phenylhydrazino; aryl groups other than phenyl, the said aryl groups being optionally substituted with one or more substituents selected from the group consisting of halogen, C₁₋₇ alkyl, C₁₋₇ alkoxy; optionally substituted heterocyclic radicals; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals selected from the group consisting of carbonyl (oxo), thiocarbonyl, alcohol (hydroxyl), thiol, ether, thio-ether, acetal, thio-acetal, amino, imino, oximino, alkyloximino, amino-acid, cyano, sulfonyl, sulfinyl, sulfonamido and halogen; branched or straight, saturated or unsaturated aliphatic chains of 1 to 7 carbon atoms optionally containing one or more functions selected from the group consisting of carbonyl (oxo), thiocarbonyl, alcohol (hydroxyl), thiol, ether, thio-ether, acetal, thio-acetal, amino, imino, oximino, alkyloximino, aminoacid, cyano, sulfonyl, sulfinyl, sulfonamido and halogen; and
R₃ is an atom or a group selected from the group consisting of fluoro, bromo, iodo, C₂₋₇ alkyl; C₂₋₇ alkenyl; C₂₋₇ alkynyl; halo C₁₋₇ alkyl; C₁₋₇ alkoxy; C₃₋₁₀ cycloalkoxy; aryloxy; arylalkyloxy; oxyheterocyclic; heterocyclic-substituted alkyloxy; thio C₂₋₇ alkyl; thio C₃₋₁₀ cycloalkyl; thioaryl; thioheterocyclic; arylalkylthio; heterocyclic-substituted alkylthio; hydroxylamino; alkoxyamino; thioalkylamino; mercaptoamino; acylamino; thio-acylamino; thio-acetal; carboxylic acid; carboxylic acid esters, thioesters, amides, halides, anhydrides and thioamides; thiocarboxylic acid; thiocarboxylic acid esters, thioesters, amides, halides, anhydrides and thioamides; hydroxyl; sulfhydryl; nitro; carbamoyl; thiocarbamoyl; ureido; thio-ureido; amino; alkylamino; cycloalkylamino; alkenylamino; cycloalkenylamino; alkynylamino; arylamino; arylalkylamino; hydroxyalkyl-amino; mercaptoalkylamino; heterocyclic amino; heterocyclic-substituted alkylamino; oximino; alkyloximino; hydrazino; alkylhydrazino; phenyl-hydrazino; cysteinyl acid, esters, thioesters, amides and thioamides thereof; aryl optionally substituted with one or more substituents selected from the group consisting of halogen, C₁₋₇ alkyl and C₁₋₇ alkoxy; optionally substituted heterocyclic radicals; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals selected from the group consisting of carbonyl (oxo), thiocarbonyl, alcohol (hydroxyl), thiol, ether, thio-ether, acetal, thio-acetal, amino, imino, oximino, alkyloximino, amino-acid, cyano, carboxylic acid or ester or thioester or amide, nitro, thio C₁₋₇ alkyl, thio C₃₋₁₀ cycloalkyl, alkylamino, sulfonyl, sulfonamido and halogen; branched or straight, saturated or unsaturated aliphatic chains of 2 to 7 carbon atoms optionally containing one or more functions selected from the group consisting of thiocarbonyl, alcohol (hydroxyl), thiol, ether, thio-ether, thio-acetal, amino, imino, oximino, alkyloximino, amino-acid, cyano, sulfonyl, sulfinyl, sulfonamido and halogen; or R₃ together with R₄ forms a homocyclic or heterocyclic radical;
and/or a pharmaceutically acceptable addition salt thereof, and/or a stereoisomer thereof, and/or a mono- or a di-N-oxide thereof, and/or a solvate thereof, and/or a dihydro- or tetrahydropteridine derivative thereof, and/or a pro-drug thereof, with the proviso that said pteridine derivative is not 2-amino-4-ethoxy-6-(4-fluorophenyl)-pteridine or 2-amino-4-isopropoxy-6-(4-fluorophenyl)-pteridine.

39. A method according to claim 38, wherein said virus is HCV.

40. A method according to claim 38, wherein said therapeutically effective amount is from 0.1 mg to 5 mg per day per kg bodyweight of said patient.

41. A method according to claim 38, wherein said administration is oral administration.

42. A method of treatment or prevention of an infection due to a virus from the Flaviridae family, consisting essentially of administering to a patient in need thereof a therapeutically effective amount of a pteridine derivative having the structural formula (III): 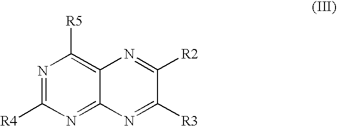
wherein:
a first group of one or more of the substituents R₂, R₃, R₄ and R₅ of the pteridine ring is independently selected from groups represented by the general formula (IV): 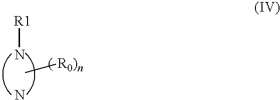
wherein: 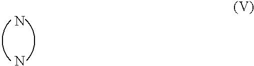
schematically represents a saturated or partly unsaturated heterocyclic ring with at least two nitrogen atoms in the said heterocyclic ring and with a total of 5 to 7 atoms in the said heterocyclic ring, and optionally with one or more other heteroatoms (e.g. oxygen or sulfur) in the said heterocyclic ring or attached to one or more carbon atoms of said heterocyclic ring (for instance in the form of a carbonyl or thiocarbonyl group), wherein one of said at least two nitrogen atoms in the heterocyclic ring is attached to a carbon atom of the pteridine ring at any of positions 2, 4, 6 or 7 of the pteridine ring, wherein the said heterocyclic ring may be fused to one or more aromatic hydrocarbon rings, and wherein:
each substituent R₀ of the heterocyclic ring (III) is a group independently selected from the group consisting of halogen and C₁₋₇ alkyl;
n is an integer from 0 to 6;
R₁ is a substituent group selected from the group consisting of formyl, acyl, thio-acyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cyclo-alkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents; and
wherein a second group of the substituents R₂, R₃, R₄ and R₅ of the pteridine ring is independently selected from the group consisting of hydrogen; halogen; C₁₋₇ alkyl; C₂₋₇ alkenyl; C₂₋₇ alkynyl; halo C₁₋₇ alkyl; carboxy C₁₋₇ alkyl; carboxyaryl; C₁₋₇ alkoxy; C₃₋₁₀ cycloalkoxy; aryloxy; arylalkyloxy; oxyheterocyclic; heterocyclic-substituted alkyloxy; thio C₁₋₇ alkyl; thio C₃₋₁₀ cycloalkyl; thioaryl; thioheterocyclic; arylalkylthio; heterocyclic-substituted alkylthio; hydroxylamino; mercapto-amino; acylamino; thio-acylamino; alkoxyamino; thioalkylamino; amino; alkylamino; cycloalkylamino; arylamino; arylalkylamino; hydroxyalkylamino; mercaptoalkylamino; heterocyclic-substituted amino; heterocyclic-substituted arylamino; heterocyclic-substituted alkylamino; aryl optionally substituted with one or more substituents; optionally substituted heterocyclic radicals; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals independently selected from the group consisting of carbonyl, thiocarbonyl, hydroxyl, thiol, ether and halogen; branched or straight, saturated or unsaturated aliphatic chains of 1 to 7 carbon atoms optionally containing one or more functions, atoms or radicals independently selected from the group consisting of halogen, carbonyl, thiocarbonyl, hydroxyl, thiol, ether and amino; or R₂ together with R₃ and the carbon atoms in positions 6 and 7 of the pteridine ring forms a homocyclic or heterocyclic radical;
and/or a pharmaceutically acceptable addition salt thereof, and/or a stereoisomer thereof, and/or a mono- or a di-N-oxide thereof, and/or a solvate thereof, and/or a dihydro- or tetrahydropteridine derivative thereof, and/or a pro-drug thereof.

43. A method according to claim 42, wherein said virus is HCV.

44. A method according to claim 42, wherein said therapeutically effective amount is from 0.1 mg to 5 mg per day per kg bodyweight of said patient.

45. A method according to claim 42, wherein said administration is oral administration.

46. A pteridine derivative represented by 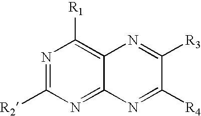
the structural formula (VI): 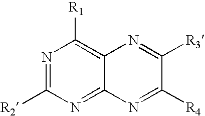
or the structural formula (VIII)
wherein:
R₁ is selected from the group consisting of hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl;
R₃ is selected from the group consisting of unsubstituted, monosubstituted and disubstituted aryl groups wherein the optional substituent(s) may be, but are not limited to, halogen, C₁₋₄ alkoxy, C₁₋₄ alkyl, aryl groups bonded to the pteridine ring via a saturated or unsaturated aliphatic spacer which may be halogenated or hydroxylated, and aliphatic substituents which may contain an ether, hydroxy, amino or C₁₋₄ alkoxy group; and
R₄ is selected from the group consisting of hydrogen, alkyl, alkoxy, substituted and non-substituted aryl groups,
R₂′ is —NH—CHR₆R₇ or —NH—R₈, wherein R₆ and R₇ are independently selected from the group consisting of hydrogen, C₁₋₆ alkyl substituted with one or more substituents selected from the group consisting of halogen and C₁₋₄ alkoxy, C₃₋₁₀ cycloalkyl, aryl and heterocyclyl, wherein said aryl is optionally substituted with one or more substituents selected from the group consisting of halogen, hydroxy, amino, nitro, cyano, trifluoromethyl, trifluoromethoxy, C₁₋₄ alkyl, C₁₋₄ alkoxy, di-C₁₋₄ alkylamino, mono-C₁₋₄ alkylamino, carboxamido, sulfamoyl, carbamoyl, sulfonamido and phenoxy, provided that R₆ and R₇ are not both hydrogen; and wherein R₈ is selected from the group consisting of C₃₋₁₀ cycloalkyl substituted at the carbon position adjacent to the N atom of R₂′ with aryl or heteroaryl wherein said aryl is optionally substituted with halogen, heteroaryl, and heteroaryl or aryl wherein said heteroaryl or aryl is substituted with one or more substituents selected from the group consisting of halogen and C₁₋₄ alkyl;
R₃′ is selected from the group consisting of halogen, heterocyclyl, and, mono-substituted or disubstituted aryl and heterocyclyl, wherein at least one substituent of said aryl is selected from the group consisting of amino, C₄₋₆ alkyl, C₄₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy, and optionally further substituted with halogen, C₁₋₄ alkyl, C₁₋₄ alkoxy; and at least one substituent of said heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁ are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
or a pharmaceutical acceptable addition salt or a stereochemical isomeric form thereof or a N-oxide thereof or a solvate thereof or a prodrug thereof.

47. A pteridine derivative represented by 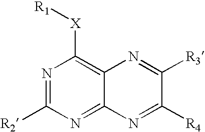
or the structural formula (X) 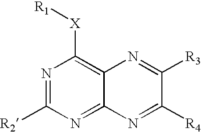
or the structural formula (XI)
wherein X represents an oxygen atom or a group with the formula S(O)ₘ wherein m is an integer from 0 to 2, or a group with the formula NZ and wherein:
R₁ is a group selected from the group consisting of C₁₋₇ alkyl, C₂₋₇ alkenyl, C₂₋₇ alkynyl, C₃₋₁₀ cycloalkyl, C₃₋₁₀ cycloalkenyl, aryl, alkylaryl, arylalkyl, heterocyclic, heterocyclic-substituted alkyl and alkyl-substituted heterocyclic, each of said groups being optionally substituted with one or more substituents selected from the group consisting of halogen, C₁₋₄ alkyl and C₁₋₄ alkoxy; or R₁ is a carboxyalkyl, carboxyaryl, thiocarboxyaryl or thiocarboxyalkyl group;
Z is a group independently defined as R₁ or Z is hydrogen or the group NZ together with R₁ is either hydroxylamino or an optionally substituted heterocyclic group containing at least one nitrogen atom;
R₄ is an atom or a group selected from the group consisting of hydrogen; halogen; C₁₋₇ alkyl; C₂₋₇ alkenyl; C₂₋₇ alkynyl; halo C₁₋₇ alkyl; carboxy C₁₋₇ alkyl; carboxyaryl; C₁₋₇ alkoxy; C₃₋₁₀ cycloalkoxy; aryloxy; arylalkyloxy; thio C₁₋₇ alkyl; thio C₃₋₁₀ cycloalkyl; thioaryl; arylalkylthio; acetal; thio-acetal; carboxylic acid; carboxylic acid esters, thioesters, halides, anhydrides, amides and thioamides; thiocarboxylic acid; thiocarboxylic acid esters, thioesters, halides, anhydrides, amides and thioamides; hydroxyl; sulfhydryl; nitro; cyano; carbamoyl; thiocarbamoyl, ureido; thio-ureido; hydrazino; alkylhydrazino; phenylhydrazino; cysteinyl acid, esters, thioesters, halides, anhydrides, amides and thioamides thereof; phenyl substituted with one or more substituents selected from the group consisting of C₁₋₇ alkyl, C₂₋₇ alkenyl, C₂₋₇ alkynyl, halo C₁₋₇ alkyl, nitro, hydroxyl, sulfhydryl, amino, C₃₋₁₀ cycloalkoxy, aryloxy, arylalkyloxy, thio C₁₋₇ alkyl, thio C₃₋₁₀ cycloalkyl, thioaryl, thioheterocyclic, arylalkylthio, formyl, carbamoyl, thiocarbamoyl, ureido, thio-ureido, sulfonamido, cyano, carboxylic acid or esters or thioesters or halides or anhydrides or amides thereof, thiocarboxylic acid or esters or thioesters or halides or anhydrides or amides thereof, hydrazino, alkylhydrazino and phenylhydrazino; aryl groups other than phenyl, the said aryl groups being optionally substituted with one or more substituents selected from the group consisting of halogen, C₁₋₇ alkyl, C₁₋₇ alkoxy; and
R₃ is an atom or a group selected from the group consisting of fluoro, bromo, iodo, C₂₋₇ alkyl; C₂₋₇ alkenyl; C₂₋₇ alkynyl; halo C₁₋₇ alkyl; C₁₋₇ alkoxy; C₃₋₁₀ cycloalkoxy; aryloxy; arylalkyloxy; oxyheterocyclic; heterocyclic-substituted alkyloxy; thio C₂₋₇ alkyl; thio C₃₋₁₀ cycloalkyl; thioaryl; thioheterocyclic; arylalkylthio; heterocyclic-substituted alkylthio; hydroxylamino; alkoxyamino; thioalkylamino; mercaptoamino; acylamino; thio-acylamino; thio-acetal; carboxylic acid; carboxylic acid esters, thioesters, amides, halides, anhydrides and thioamides; thiocarboxylic acid; thiocarboxylic acid esters, thioesters, amides, halides, anhydrides and thioamides; hydroxyl; sulfhydryl; nitro; carbamoyl; thiocarbamoyl; ureido; thio-ureido; amino; alkylamino; cycloalkylamino; alkenylamino; cycloalkenylamino; alkynylamino; arylamino; arylalkylamino; hydroxyalkyl-amino; mercaptoalkylamino; heterocyclic amino; heterocyclic-substituted alkylamino; oximino; alkyloximino; hydrazino; alkylhydrazino; phenyl-hydrazino; cysteinyl acid, esters, thioesters, amides and thioamides thereof; aryl optionally substituted with one or more substituents selected from the group consisting of halogen, C₁₋₇ alkyl and C₁₋₇ alkoxy; optionally substituted heterocyclic radicals; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals selected from the group consisting of carbonyl (oxo), thiocarbonyl, alcohol (hydroxyl), thiol, ether, thio-ether, acetal, thio-acetal, amino, imino, oximino, alkyloximino, amino-acid, cyano, carboxylic acid or ester or thioester or amide, nitro, thio C₁₋₇ alkyl, thio C₃₋₁₀ cycloalkyl, alkylamino, sulfonyl, sulfonamido and halogen; branched or straight, saturated or unsaturated aliphatic chains of 2 to 7 carbon atoms optionally containing one or more functions selected from the group consisting of thiocarbonyl, alcohol (hydroxyl), thiol, ether, thio-ether, thio-acetal, amino, imino, oximino, alkyloximino, amino-acid, cyano, sulfonyl, sulfinyl, sulfonamido and halogen; or R₃ together with R₄ forms a homocyclic or heterocyclic radical;
R₂′ is —NH—CHR₆R₇ or —NH—R₈, wherein R₆ and R₇ are independently selected from the group consisting of hydrogen, C₁₋₆ alkyl substituted with one or more substituents selected from the group consisting of halogen and C₁₋₄ alkoxy, C₃₋₁₀ cycloalkyl, heterocyclyl, and aryl substituted with one or more substituents selected from the group consisting of halogen, hydroxy, amino, nitro, cyano, trifluoromethyl, trifluoromethoxy, C₁₋₄ alkyl, C₁₋₄ alkoxy, di-C₁₋₄ alkylamino, mono-C₁₋₄ alkylamino, carboxamido, sulfamoyl, carbamoyl, sulfonamido and phenoxy, provided that R₆ and R₇ are not both hydrogen; and wherein R₈ is selected from the group consisting of C₃₋₁₀ cycloalkyl optionally substituted at the carbon position adjacent to the N atom of R₂′ with aryl or heteroaryl wherein said aryl is optionally substituted with halogen, heteroaryl, and heteroaryl or aryl wherein said heteroaryl or aryl is substituted with one or more substituents selected from the group consisting of halogen and C₁₋₄ alkyl;
R₃′ is selected from the group consisting of halogen, heterocyclyl, and, mono-substituted or disubstituted aryl and heterocyclyl, wherein at least one substituent of said aryl is selected from the group consisting of amino, C₄₋₆ alkyl, C₄₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy, and optionally further substituted with halogen, C₁₋₄ alkyl, C₁₋₄ alkoxy; and at least one substituent of said heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁ are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
or a pharmaceutical acceptable addition salt or a stereochemical isomeric form thereof or a N-oxide thereof or a solvate thereof or a prodrug thereof.

48. A pteridine derivative represented by 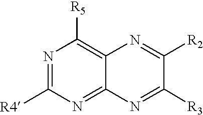
or the structural formula (XIII) 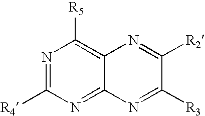
or the structural formula (XIV)
wherein
a first group of one or more of the substituents R₂, R₃ and R₅ of the pteridine ring is independently selected from groups represented by the general formula (IV): 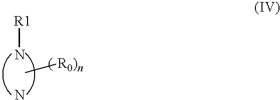
wherein: 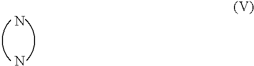
schematically represents a saturated or partly unsaturated heterocyclic ring with at least two nitrogen atoms in the said heterocyclic ring and with a total of 5 to 7 atoms in the said heterocyclic ring, and optionally with one or more other heteroatoms (e.g. oxygen or sulfur) in the said heterocyclic ring or attached to one or more carbon atoms of said heterocyclic ring (for instance in the form of a carbonyl or thiocarbonyl group), wherein one of said at least two nitrogen atoms in the heterocyclic ring is attached to a carbon atom of the pteridine ring at any of positions 2, 4, 6 or 7 of the pteridine ring, wherein the said heterocyclic ring may be fused to one or more aromatic hydrocarbon rings, and wherein:
each substituent R₀ of the heterocyclic ring (III) is a group independently selected from the group consisting of halogen and C₁₋₇ alkyl;
n is an integer from 0 to 6;
R₁ is a substituent group selected from the group consisting of formyl, acyl, thio-acyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cyclo-alkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents; and
wherein a second group of the substituents R₂, R₃ and R₅ of the pteridine ring is independently selected from the group consisting of hydrogen; halogen; C₁₋₇ alkyl; C₂₋₇ alkenyl; C₂₋₇ alkynyl; halo C₁₋₇ alkyl; carboxy C₁₋₇ alkyl; carboxyaryl; C₁₋₇ alkoxy; C₃₋₁₀ cycloalkoxy; aryloxy; arylalkyloxy; thio C₁₋₇ alkyl; thio C₃₋₁₀ cycloalkyl; thioaryl; arylalkylthio; hydroxylamino; mercapto-amino; acylamino; thio-acylamino; alkoxyamino; thioalkylamino; amino; alkylamino; cycloalkylamino; arylamino; arylalkylamino; hydroxyalkylamino; mercaptoalkylamino; aryl optionally substituted with one or more substituents; aromatic or heterocyclic substituents substituted with an aliphatic spacer between the pteridine ring and the aromatic or heterocyclic substituent, whereby said aliphatic spacer is a branched or straight, saturated or unsaturated aliphatic chain of 1 to 4 carbon atoms which may contain one or more functions, atoms or radicals independently selected from the group consisting of carbonyl, thiocarbonyl, hydroxyl, thiol, ether and halogen; branched or straight, saturated or unsaturated aliphatic chains of 1 to 7 carbon atoms optionally containing one or more functions, atoms or radicals independently selected from the group consisting of halogen, carbonyl, thiocarbonyl, hydroxyl, thiol, ether and amino; or R₂ together with R₃ and the carbon atoms in positions 6 and 7 of the pteridine ring forms a homocyclic or heterocyclic radical;
and wherein
R₄′ is —NH—CHR₆R₇ or —NH—R₈, wherein R₆ and R₇ are independently selected from the group consisting of hydrogen, C₁₋₆ alkyl substituted with one or more substituents selected from the group consisting of halogen and C₁₋₄ alkoxy, C₃₋₁₀ cycloalkyl, heterocyclyl, and aryl substituted with one or more substituents selected from the group consisting of halogen, hydroxy, amino, nitro, cyano, trifluoromethyl, trifluoromethoxy, C₁₋₄ alkyl, C₁₋₄ alkoxy, di-C₁₋₄ alkylamino, mono-C₁₋₄ alkylamino, carboxamido, sulfamoyl, carbamoyl, sulfonamido and phenoxy, provided that R₆ and R₇ are not both hydrogen; and wherein R₈ is selected from the group consisting of C₃₋₁₀ cycloalkyl optionally substituted at the carbon position adjacent to the N atom of R₄′ with aryl or heteroaryl wherein said aryl is optionally substituted with halogen, heteroaryl, and heteroaryl or aryl wherein said heteroaryl or aryl is substituted with one or more substituents selected from the group consisting of halogen and C₁₋₄ alkyl;
R₂′ is selected from the group consisting of halogen, heterocyclyl, and, mono-substituted or disubstituted aryl and heterocyclyl, wherein at least one substituent of said aryl or heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one or more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁ are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
or a pharmaceutical acceptable addition salt or a stereochemical isomeric form thereof or a N-oxide thereof or a solvate thereof or a prodrug thereof.

49. A pharmaceutical composition comprising at least one pteridine derivative according to claim 46.

50. A pharmaceutical composition comprising at least one pteridine derivative according to claim 47.

51. A pharmaceutical composition comprising at least one pteridine derivative according to claim 48.

52. A method of treatment or prevention of an infection due to a virus from the Flaviviridae family, by administering to a patient in need thereof a therapeutically effective amount of a pteridine derivative according to claim 46.

53. A method of treatment or prevention of an infection due to a virus from the Flaviviridae family, by administering to a patient in need thereof a therapeutically effective amount of a pteridine derivative according to claim 47.

54. A method of treatment or prevention of an infection due to a virus from the Flaviviridae family, by administering to a patient in need thereof a therapeutically effective amount of a pteridine derivative according to claim 48.

55. A method of treatment or prevention of an infection due to a virus from the Flaviviridae family, by administering to a patient in need thereof a therapeutically effective amount of a pteridine derivative according to 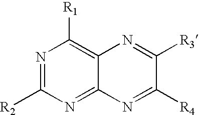
the structural formula (VII)
wherein:
R₁ is selected from the group consisting of hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl;
R₂ is selected from the group consisting of amino, hydroxylamino, (mono- or di) C₁₋₇ alkylamino, (mono- or di) C₁₋₇ alkyloxyamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy or a saturated or unsaturated heterocyclic compound containing at least one nitrogen and optionally substituted by one or more C₁₋₄ alkyl, hydroxy C₁₋₄ alkyl, C₁₋₄ alkyloxy, halo, hydroxy, hydroxy-carbonyl, and C₁₋₄ alkyloxycarbonyl,
R₄ is selected from the group consisting of hydrogen, alkyl, alkoxy, substituted and non-substituted aryl groups,
R₃′ is selected from the group consisting of halogen, heterocyclyl, and, mono-substituted or disubstituted aryl and heterocyclyl, wherein at least one substituent of said aryl is selected from the group consisting of amino, C₄₋₆ alkyl, C₄₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy, and optionally further substituted with halogen, C₁₋₄ alkyl, C₁₋₄ alkoxy; and at least one substituent of said heterocyclyl is selected from the group consisting of halogen, amino, C₁₋₆ alkyl, C₁₋₆ alkoxy, —CONHR₉, —NR₁₂COR₁₀, —NR₁₂SO₂R₁₁, —SO₂NH₂, heterocyclyl and heterocyclyl substituted with one or more substituents selected from the group consisting of hydroxy, oxo, halogen, amino, C₁₋₆ alkyl and C₁₋₆ alkoxy;
R₉ is selected from the group consisting of H, C₃₋₁₀ cycloalkyl optionally substituted with one more substituents selected from the group consisting of cyano, halogen, hydroxy, amino, C₁₋₆ alkyl and C₁₋₆ alkoxy; C₁₋₆ alkyl optionally substituted with one or more substituents selected from the group consisting of amino, alkylamino, cyano, dialkylamino, halogen, and heterocyclyl; C₁₋₆ alkoxy; heterocyclyl optionally substituted with C₁₋₆ alkyl; and phenyl optionally substituted with one or more halogens;
R₁₀ and R₁₁, are each independently selected from the group consisting of C₁₋₆ alkyl optionally substituted with one or more substituents selected from the group consisting of amino, cyano, halogen and hydroxy; C₁₋₆ alkoxy optionally substituted with one or more substituents selected from the group consisting of amino, alkylamino, cyano, dialkylamino, halogen, and heterocyclyl; heterocyclyl optionally substituted with one or more substituents selected from the group consisting of C₁₋₆ alkyl, acylamino and oxo; C₃₋₁₀ cycloalkyl optionally substituted with one or more substituents selected from the group consisting of amino or hydroxy; and amino optionally substituted with one or more substituents selected from the group consisting of C₁₋₆ alkyl wherein said C₁₋₆ alkyl is optionally substituted with one or more substituents selected from the group consisting of amino, alkylamino, cyano, dialkylamino, halogen and heterocyclyl;
R₁₂ is selected from the group consisting of H and C₁₋₆ alkyl, wherein said C₁₋₆ alkyl is optionally substituted with one or more substituents selected from the group consisting of cyano, halogen and hydroxy;
or a pharmaceutical acceptable addition salt or a stereochemical isomeric form thereof or a N-oxide thereof or a solvate thereof or a prodrug thereof.

56. A method according to claim 55, wherein said administration is oral.

57. A method according to claim 55, wherein said virus is HCV.

58. A method according to claim 55, wherein said therapeutically effective amount is from 0.1 mg to 5 mg per day per kg bodyweight of said patient.

59. A method according to claim 54, wherein said therapeutically effective amount is from 0.1 mg to 5 mg per day per kg bodyweight of said patient.

60. A pteridine derivative selected from the group consisting of:
4-ethoxy-6-(4-acetamidophenyl)-pteridin-2-yl]-(4-fluoro-benzyl)-amine,
4-ethoxy-6-(3-acetamidophenyl)-pteridin-2-yl]-(4-fluoro-benzyl)-amine,
4-ethoxy-6-(4-acetylphenyl)-pteridin-2-yl]-(4-fluoro-benzyl)-amine,
4-ethoxy-6-(3-acetylphenyl)-pteridin-2-yl]-(4-fluoro-benzyl)-amine,
4-ethoxy-6-(2-acetylphenyl)-pteridin-2-yl]-(4-fluoro-benzyl)-amine,
4-ethoxy-6-(5-acetyl-2-chlorophenyl)-pteridin-2-yl]-(4-fluoro-benzyl)-amine,
4-ethoxy-6-(4-acetyl-3-fluorophenyl)-pteridin-2-yl]-(4-fluoro-benzyl)-amine,
4-ethoxy-6-(5-acetyl-2-fluorophenyl)-pteridin-2-yl]-(4-fluoro-benzyl)-amine,
4-ethoxy-6-(3-aminophenyl)-pteridin-2-yl]-(4-fluoro-benzyl)-amine,
4-ethoxy-6-(4-aminomethylphenyl)-pteridin-2-yl]-(4-fluoro-benzyl)-amine,
4-ethoxy-6-(4-benzyloxy-2-fluorophenyl)-pteridin-2-yl]-(4-fluoro-benzyl)-amine,
4-ethoxy-6-(4-benzyloxy-3-fluorophenyl)-pteridin-2-yl]-(4-fluoro-benzyl)-amine,
4-ethoxy-6-(4-bromophenyl)-pteridin-2-yl]-(4-fluoro-benzyl)-amine,
4-ethoxy-6-(3-bromophenyl)-pteridin-2-yl]-(4-fluoro-benzyl)-amine,
4-ethoxy-6-(4-bromo-2,5-dimethylphenyl)-pteridin-2-yl]-(4-fluoro-benzyl)-amine,
4-ethoxy-6-(2-bromo-5-fluorophenyl)-pteridin-2-yl]-(4-fluoro-benzyl)-amine,
4-ethoxy-6-(2-bromo-6-fluorophenyl)-pteridin-2-yl]-(4-fluoro-benzyl)-amine,
4-ethoxy-6-(2-carboxy-5-fluorophenyl)-pteridin-2-yl]-(4-fluoro-benzyl)-amine,
4-ethoxy-6-(2-carboxyphenyl)-pteridin-2-yl]-(4-fluoro-benzyl)-amine,
4-ethoxy-6-(4-carboxyphenyl)-pteridin-2-yl]-(4-fluoro-benzyl)-amine,
4-ethoxy-6-(4-carboxy-2-chlorophenyl)-pteridin-2-yl]-(4-fluoro-benzyl)-amine,
4-ethoxy-6-(5-carboxy-2-chlorophenyl)-pteridin-2-yl]-(4-fluoro-benzyl)-amine,
4-ethoxy-6-(4-carboxy-3-chlorophenyl)-pteridin-2-yl]-(4-fluoro-benzyl)-amine,
4-ethoxy-6-(3-carboxyphenyl)-pteridin-2-yl]-(4-fluoro-benzyl)-amine,
4-ethoxy-6-(2-chloro-5-formylphenyl)-pteridin-2-yl]-(4-fluoro-benzyl)-amine,
4-ethoxy-6-(2-chloro-5-hydroxyphenyl)-pteridin-2-yl]-(4-fluoro-benzyl)-amine,
4-ethoxy-6-(3-chloro-4-fluorophenyl)-pteridin-2-yl]-(4-fluoro-benzyl)-amine,
4-ethoxy-6-(2-chloro-4-fluorophenyl)-pteridin-2-yl]-(4-fluoro-benzyl)-amine,
4-ethoxy-6-(4-chloro-2-fluorophenyl)-pteridin-2-yl]-(4-fluoro-benzyl)-amine,
4-ethoxy-6-(3-chloro-5-methoxyphenyl)-pteridin-2-yl]-(4-fluoro-benzyl)-amine,
4-ethoxy-6-(2-chloro-4-methylphenyl)-pteridin-2-yl]-(4-fluoro-benzyl)-amine,
4-ethoxy-6-(2-chloro-5-methylphenyl)-pteridin-2-yl]-(4-fluoro-benzyl)-amine,
4-ethoxy-6-(3-chloro-5-trifluoromethylphenyl)-pteridin-2-yl]-(4-fluoro-benzyl)-amine,
4-ethoxy-6-(2-chloro-5-trifluoromethoxyphenyl)-pteridin-2-yl]-(4-fluoro-benzyl)-amine,
4-ethoxy-6-(4-chloro-2-trifluoromethylphenyl)-pteridin-2-yl]-(4-fluoro-benzyl)-amine,
4-ethoxy-6-(4-chlorophenyl)-pteridin-2-yl]-(4-fluoro-benzyl)-amine,
4-ethoxy-6-(3-chlorophenyl)-pteridin-2-yl]-(4-fluoro-benzyl)-amine,
4-ethoxy-6-(2-chlorophenyl)-pteridin-2-yl]-(4-fluoro-benzyl)-amine,
4-ethoxy-6-(4-cyanophenyl)-pteridin-2-yl]-(4-fluoro-benzyl)-amine,
4-ethoxy-6-(3-cyanophenyl)-pteridin-2-yl]-(4-fluoro-benzyl)-amine,
4-ethoxy-6-(2-cyanophenyl)-pteridin-2-yl]-(4-fluoro-benzyl)-amine,
4-ethoxy-6-(2,6-dichlorophenyl)-pteridin-2-yl]-(4-fluoro-benzyl)-amine,
4-ethoxy-6-(3,4-dichlorophenyl)-pteridin-2-yl]-(4-fluoro-benzyl)-amine,
4-ethoxy-6-(2,6-dichlorophenyl)-pteridin-2-yl]-(4-fluoro-benzyl)-amine,
4-ethoxy-6-(2,4-dichlorophenyl)-pteridin-2-yl]-(4-fluoro-benzyl)-amine,
4-ethoxy-6-(2,3-dichlorophenyl)-pteridin-2-yl]-(4-fluoro-benzyl)-amine,
4-ethoxy-6-(3,5-dichlorophenyl)-pteridin-2-yl]-(4-fluoro-benzyl)-amine,
4-ethoxy-6-(3,5-difluorophenyl)-pteridin-2-yl]-(4-fluoro-benzyl)-amine,
4-ethoxy-6-(3,4-difluorophenyl)-pteridin-2-yl]-(4-fluoro-benzyl)-amine,
4-ethoxy-6-(2,6-difluorophenyl)-pteridin-2-yl]-(4-fluoro-benzyl)-amine,
4-ethoxy-6-(2,5-difluorophenyl)-pteridin-2-yl]-(4-fluoro-benzyl)-amine,
4-ethoxy-6-(2,4-difluorophenyl)-pteridin-2-yl]-(4-fluoro-benzyl)-amine,
4-ethoxy-6-(2,3-difluorophenyl)-pteridin-2-yl]-(4-fluoro-benzyl)-amine,
4-ethoxy-6-(4-[N,N-dimethylamino]phenyl)-pteridin-2-yl]-(4-fluoro-benzyl)-amine,
4-ethoxy-6-(2-[N,N-dimethylaminomethyl]phenyl)-pteridin-2-yl]-(4-fluoro-benzyl)-amine,
4-ethoxy-6-(3,5-dimethyl phenyl)-pteridin-2-yl]-(4-fluorobenzyl)-amine,
4-ethoxy-6-(3,4-dimethylphenyl)-pteridin-2-yl]-(4-fluorobenzyl)-amine,
4-ethoxy-6-(2,6-dimethylphenyl)-pteridin-2-yl]-(4-fluorobenzyl)-amine,
4-ethoxy-6-(2,6-dimethoxyphenyl)-pteridin-2-yl]-(4-fluorobenzyl)-amine,
4-ethoxy-6-(2,5-dimethoxyphenyl)-pteridin-2-yl]-(4-fluorobenzyl)-amine,
4-ethoxy-6-(2,4-dimethoxyphenyl)-pteridin-2-yl]-(4-fluorobenzyl)-amine,
4-ethoxy-6-(4-ethoxyphenyl)-pteridin-2-yl]-(4-fluorobenzyl)-amine,
4-ethoxy-6-(2-ethoxyphenyl)-pteridin-2-yl]-(4-fluorobenzyl)-amine,
4-ethoxy-6-(4-ethoxycarbonylphenyl)-pteridin-2-yl]-(4-fluorobenzyl)-amine,
4-ethoxy-6-(3-ethoxycarbonylphenyl)-pteridin-2-yl]-(4-fluorobenzyl)-amine,
4-ethoxy-6-(4-ethylphenyl)-pteridin-2-yl]-(4-fluorobenzyl)-amine,
4-ethoxy-6-(4-fluorophenyl)-pteridin-2-yl]-(4-fluorobenzyl)-amine,
4-ethoxy-6-(3-fluorophenyl)-pteridin-2-yl]-(4-fluorobenzyl)-amine,
4-ethoxy-6-(2-fluorophenyl)-pteridin-2-yl]-(4-fluorobenzyl)-amine,
4-ethoxy-6-(3-fluoro-4-formylphenyl)-pteridin-2-yl]-(4-fluorobenzyl)-amine,
4-ethoxy-6-(4-fluoro-3-formylphenyl)-pteridin-2-yl]-(4-fluorobenzyl)-amine,
4-ethoxy-6-(4-fluoro-2-methylphenyl)-pteridin-2-yl]-(4-fluorobenzyl)-amine,
4-ethoxy-6-(2-fluoro-5-methylphenyl)-pteridin-2-yl]-(4-fluorobenzyl)-amine,
4-ethoxy-6-(2-fluoro-5-methoxyphenyl)-pteridin-2-yl]-(4-fluorobenzyl)-amine,
4-ethoxy-6-(5-fluoro-2-methoxycarbonylphenyl)-pteridin-2-yl]-(4-fluorobenzyl)-amine,
4-ethoxy-6-(2-formyl-5-methoxyphenyl)-pteridin-2-yl]-(4-fluorobenzyl)-amine,
4-ethoxy-6-(5-formyl-2-methoxyphenyl)-pteridin-2-yl]-(4-fluorobenzyl)-amine,
4-ethoxy-6-(2-formyl-5-methylphenyl)-pteridin-2-yl]-(4-fluorobenzyl)-amine,
4-ethoxy-6-(2-formylphenyl)-pteridin-2-yl]-(4-fluorobenzyl)-amine,
4-ethoxy-6-(3-formylphenyl)-pteridin-2-yl]-(4-fluorobenzyl)-amine,
4-ethoxy-6-(4-formylphenyl)-pteridin-2-yl]-(4-fluorobenzyl)-amine,
4-ethoxy-6-(3-hydroxy-4-methoxycarbonylphenyl)-pteridin-2-yl]-(4-fluorobenzyl)-amine,
4-ethoxy-6-(3-hydroxymethylphenyl)-pteridin-2-yl]-(4-fluorobenzyl)-amine,
4-ethoxy-6-(4-hydroxymethylphenyl)-pteridin-2-yl]-(4-fluorobenzyl)-amine,
4-ethoxy-6-(4-hydroxyphenyl)-pteridin-2-yl]-(4-fluorobenzyl)-amine,
4-ethoxy-6-(3-hydroxyphenyl)-pteridin-2-yl]-(4-fluorobenzyl)-amine,
4-ethoxy-6-(4-iodophenyl)-pteridin-2-yl]-(4-fluorobenzyl)-amine,
4-ethoxy-6-(3-iodophenyl)-pteridin-2-yl]-(4-fluorobenzyl)-amine,
4-ethoxy-6-(4-isopropoxycarbonylphenyl)-pteridin-2-yl]-(4-fluorobenzyl)-amine,
4-ethoxy-6-(3-isopropoxycarbonylphenyl)-pteridin-2-yl]-(4-fluorobenzyl)-amine,
4-ethoxy-6-(4-methanesulfonylphenyl)-pteridin-2-yl]-(4-fluorobenzyl)-amine,
4-ethoxy-6-(4-methoxy-2-formylphenyl)-pteridin-2-yl]-(4-fluorobenzyl)-amine,
4-ethoxy-6-(4-methoxycarbonylphenyl)-pteridin-2-yl]-(4-fluorobenzyl)-amine,
4-ethoxy-6-(3-methoxycarbonylphenyl)-pteridin-2-yl]-(4-fluorobenzyl)-amine,
4-ethoxy-6-(4-methoxyphenyl)-pteridin-2-yl]-(4-fluorobenzyl)-amine,
4-ethoxy-6-(3-methoxyphenyl)-pteridin-2-yl]-(4-fluorobenzyl)-amine,
4-ethoxy-6-(2-methoxyphenyl)-pteridin-2-yl]-(4-fluorobenzyl)-amine,
4-ethoxy-6-(3,4-methylenedioxyphenyl)-pteridin-2-yl]-(4-fluorobenzyl)-amine,
4-ethoxy-6-(4-methylphenyl)-pteridin-2-yl]-(4-fluorobenzyl)-amine,
4-ethoxy-6-(2-methylphenyl)-pteridin-2-yl]-(4-fluorobenzyl)-amine,
4-ethoxy-6-(4-(methylthio)phenyl)-pteridin-2-yl]-(4-fluorobenzyl)-amine,
4-ethoxy-6-(3-(methylthio)phenyl)-pteridin-2-yl]-(4-fluorobenzyl)-amine,
4-ethoxy-6-(4-morpholinophenyl)-pteridin-2-yl]-(4-fluorobenzyl)-amine,
4-ethoxy-6-(3-nitrophenyl)-pteridin-2-yl]-(4-fluorobenzyl)-amine,
4-ethoxy-6-(4-phenoxyphenyl)-pteridin-2-yl]-(4-fluorobenzyl)-amine,
4-ethoxy-6-(4-(tert-butoxycarbonyl)phenyl)-pteridin-2-yl]-(4-fluoro-benzyl)-amine,
4-ethoxy-6-(3-(tert-butoxycarbonyl)phenyl)-pteridin-2-yl]-(4-fluoro-benzyl)-amine,
4-ethoxy-6-(2-(tert-butoxycarbonyl)phenyl)-pteridin-2-yl]-(4-fluoro-benzyl)-amine,
4-ethoxy-6-(4-(tert-butoxycarbonylamino)-3-methoxyphenyl)-pteridin-2-yl]-(4-fluorobenzyl)-amine,
4-ethoxy-6-(4-(tert-butylphenyl)-pteridin-2-yl]-(4-fluorobenzyl)-amine,
4-ethoxy-6-(4-(2-thienyl)phenyl)-pteridin-2-yl]-(4-fluorobenzyl)-amine,
4-ethoxy-6-(4-trifluoromethoxyphenyl)-pteridin-2-yl]-(4-fluorobenzyl)-amine,
4-ethoxy-6-(2,4,6-trimethylphenyl)-pteridin-2-yl]-(4-fluorobenzyl)-amine,
4-ethoxy-6-(3,4,5-trifluorophenyl)-pteridin-2-yl]-(4-fluorobenzyl)-amine,
4-ethoxy-6-(3-trifluoromethoxyphenyl)-pteridin-2-yl]-(4-fluorobenzyl)-amine,
4-ethoxy-6-(3-trifluoromethylphenyl)-pteridin-2-yl]-(4-fluorobenzyl)-amine,
4-ethoxy-6-(2-trifluoromethylphenyl)-pteridin-2-yl]-(4-fluorobenzyl)-amine,
4-ethoxy-6-(3,4,5-trimethoxyphenyl)-pteridin-2-yl]-(4-fluorobenzyl)-amine,
4-ethoxy-6-(4-vinylphenyl)-pteridin-2-yl]-(4-fluorobenzyl)-amine,
4-ethoxy-6-(2-acetamidopyridin-5-yl)-pteridin-2-yl]-(4-fluorobenzyl)-amine,
4-ethoxy-6-(2-benzothienyl)-pteridin-2-yl]-(4-fluorobenzyl)-amine,
4-ethoxy-6-(1-benzothiophen-3-yl)-pteridin-2-yl]-(4-fluorobenzyl)-amine,
4-ethoxy-6-(1-benzothiophen-2-yl)-pteridin-2-yl]-(4-fluorobenzyl)-amine,
4-ethoxy-6-(2-bromo-3-chloropyridin-4-yl)-pteridin-2-yl]-(4-fluoro-benzyl)-amine,
4-ethoxy-6-(2-bromo-3-methylpyridin-5-yl)-pteridin-2-yl]-(4-fluoro-benzyl)-amine,
4-ethoxy-6-(2-bromopyridin-5-yl)-pteridin-2-yl]-(4-fluorobenzyl)-amine,
4-ethoxy-6-(2-chloro-3-methylpyridin-5-yl)-pteridin-2-yl]-(4-fluoro-benzyl)-amine,
4-ethoxy-6-(2-chloro-6-isopropylpyridin-3-yl)-pteridin-2-yl]-(4-fluoro-benzyl)-amine,
4-ethoxy-6-(5-chlorothien-2-yl)-pteridin-2-yl]-(4-fluoro-benzyl)-amine,
4-ethoxy-6-(5-bromothien-2-yl)-pteridin-2-yl]-(4-fluoro-benzyl)-amine,
4-ethoxy-6-(2-chloro-3-fluoropyridin-4-yl)-pteridin-2-yl]-(4-fluoro-benzyl)-amine,
4-ethoxy-6-(2,5-dibromopyridin-3-yl)-pteridin-2-yl]-(4-fluoro-benzyl)-amine,
4-ethoxy-6-(2,6-dichloropyridin-3-yl)-pteridin-2-yl]-(4-fluoro-benzyl)-amine,
4-ethoxy-6-(2,4-dimethoxypyrimidin-5-yl)-pteridin-2-yl]-(4-fluoro-benzyl)-amine,
4-ethoxy-6-(3,5-dimethylisoxazol-4-yl)-pteridin-2-yl]-(4-fluoro-benzyl)-amine,
4-ethoxy-6-(2,4-dioxo-1,2,3,4-tetrahydropyrimidin-5-yl)-pteridin-2-yl]-(4-fluoro-benzyl)-amine,
4-ethoxy-6-(2-ethoxypyridin-3-yl)-pteridin-2-yl]-(4-fluorobenzyl)-amine,
4-ethoxy-6-(2-fluoro-3-methylpyridin-5-yl)-pteridin-2-yl]-(4-fluorobenzyl)-amine,
4-ethoxy-6-(2-fluoropyridin-3-yl)-pteridin-2-yl]-(4-fluorobenzyl)-amine,
4-ethoxy-6-(2-fluoropyridin-5-yl)-pteridin-2-yl]-(4-fluorobenzyl)-amine,
4-ethoxy-6-(5-formyl-2-furyl)-pteridin-2-yl]-(4-fluorobenzyl)-amine,
4-ethoxy-6-(5-formyl-thiophen-2-yl)-pteridin-2-yl]-(4-fluorobenzyl)-amine,
4-ethoxy-6-(furan-3-yl)-pteridin-2-yl]-(4-fluorobenzyl)-amine,
4-ethoxy-6-(furan-2-yl)-pteridin-2-yl]-(4-fluorobenzyl)-amine,
4-ethoxy-6-(indol-5-yl)-pteridin-2-yl]-(4-fluorobenzyl)-amine,
4-ethoxy-6-(isoquinolin-4-yl)-pteridin-2-yl]-(4-fluorobenzyl)-amine,
4-ethoxy-6-(5-methylfuran-2-yl)-pteridin-2-yl]-(4-fluorobenzyl)-amine,
4-ethoxy-6-(methoxypyrimidin-5-yl)-pteridin-2-yl]-(4-fluorobenzyl)-amine,
4-ethoxy-6-(5-methyl-1-benzothiophen-2-yl)-pteridin-2-yl]-(4-fluorobenzyl)-amine,
4-ethoxy-6-(5-methyl-3-phenyl-4-isoxazolyl)-pteridin-2-yl]-(4-fluorobenzyl)-amine,
4-ethoxy-6-(3-methylpyridin-2-yl)-pteridin-2-yl]-(4-fluorobenzyl)-amine,
4-ethoxy-6-(5-methylpyridin-2-yl)-pteridin-2-yl]-(4-fluorobenzyl)-amine,
4-ethoxy-6-(5-methylpyridin-3-yl)-pteridin-2-yl]-(4-fluorobenzyl)-amine,
4-ethoxy-6-(2-methoxypyridin-3-yl)-pteridin-2-yl]-(4-fluorobenzyl)-amine,
4-ethoxy-6-(2-methoxypyridin-5-yl)-pteridin-2-yl]-(4-fluorobenzyl)-amine,
4-ethoxy-6-(pyridin-4-yl)-pteridin-2-yl]-(4-fluorobenzyl)-amine,
4-ethoxy-6-(pyridin-3-yl)-pteridin-2-yl]-(4-fluorobenzyl)-amine,
4-ethoxy-6-(pyrimidin-5-yl)-pteridin-2-yl]-(4-fluorobenzyl)-amine,
4-ethoxy-6-(quinolin-3-yl)-pteridin-2-yl]-(4-fluorobenzyl)-amine,
4-ethoxy-6-(quinolin-8-yl)-pteridin-2-yl]-(4-fluorobenzyl)-amine,
4-ethoxy-6-(thien-2-yl)-pteridin-2-yl]-(4-fluorobenzyl)-amine,
4-ethoxy-6-(4-fluorophenyl)-pteridin-2-yl]-benzylamine,
4-ethoxy-6-(4-fluorophenyl)-pteridin-2-yl]-(4-methylbenzyl)-amine,
4-ethoxy-6-(4-fluorophenyl)-pteridin-2-yl]-(3-methylbenzyl)-amine,
4-ethoxy-6-(4-fluorophenyl)-pteridin-2-yl]-(2-methylbenzyl)-amine,
4-ethoxy-6-(4-fluorophenyl)-pteridin-2-yl]-(4-methoxybenzyl)-amine,
4-ethoxy-6-(4-fluorophenyl)-pteridin-2-yl]-(3-methoxybenzyl)-amine,
4-ethoxy-6-(4-fluorophenyl)-pteridin-2-yl]-(2-methoxybenzyl)-amine,
4-ethoxy-6-(4-fluorophenyl)-pteridin-2-yl]-(2,5-dihydroxybenzyl)-amine,
4-ethoxy-6-(4-fluorophenyl)-pteridin-2-yl]-(4-propoxybenzyl)-amine,
4-ethoxy-6-(4-fluorophenyl)-pteridin-2-yl]-(4-phenoxybenzyl)-amine,
4-ethoxy-6-(4-fluorophenyl)-pteridin-2-yl]-(3-[3,4-dichlorophenoxy]-benzyl)-amine,
4-ethoxy-6-(4-fluorophenyl)-pteridin-2-yl]-(3-[3,5-dichlorophenoxy]-benzyl)-amine,
4-ethoxy-6-(4-fluorophenyl)-pteridin-2-yl]-(2-bromobenzyl)-amine,
4-ethoxy-6-(4-fluorophenyl)-pteridin-2-yl]-(3-bromobenzyl)-amine,
4-ethoxy-6-(4-fluorophenyl)-pteridin-2-yl]-(4-bromobenzyl)-amine,
4-ethoxy-6-(4-fluorophenyl)-pteridin-2-yl]-(2-chlorobenzyl)-amine,
4-ethoxy-6-(4-fluorophenyl)-pteridin-2-yl]-(3-chlorobenzyl)-amine,
4-ethoxy-6-(4-fluorophenyl)-pteridin-2-yl]-(4-chlorobenzyl)-amine,
4-ethoxy-6-(4-fluorophenyl)-pteridin-2-yl]-(2-fluorobenzyl)-amine,
4-ethoxy-6-(4-fluorophenyl)-pteridin-2-yl]-(3-fluorobenzyl)-amine,
4-ethoxy-6-(4-fluorophenyl)-pteridin-2-yl]-(2,3-dichlorobenzyl)-amine,
4-ethoxy-6-(4-fluorophenyl)-pteridin-2-yl]-(2,4-dichlorobenzyl)-amine,
4-ethoxy-6-(4-fluorophenyl)-pteridin-2-yl]-(2,6-dichlorobenzyl)-amine,
4-ethoxy-6-(4-fluorophenyl)-pteridin-2-yl]-(3,4-dichlorobenzyl)-amine,
4-ethoxy-6-(4-fluorophenyl)-pteridin-2-yl]-(3,5-dichlorobenzyl)-amine,
4-ethoxy-6-(4-fluorophenyl)-pteridin-2-yl]-(2,3-difluorobenzyl)-amine,
4-ethoxy-6-(4-fluorophenyl)-pteridin-2-yl]-(2,4-difluorobenzyl)-amine,
4-ethoxy-6-(4-fluorophenyl)-pteridin-2-yl]-(2,5-difluorobenzyl)-amine,
4-ethoxy-6-(4-fluorophenyl)-pteridin-2-yl]-(2,6-difluorobenzyl)-amine,
4-ethoxy-6-(4-fluorophenyl)-pteridin-2-yl]-(3,4-difluorobenzyl)-amine,
4-ethoxy-6-(4-fluorophenyl)-pteridin-2-yl]-(3,5-difluorobenzyl)-amine,
4-ethoxy-6-(4-fluorophenyl)-pteridin-2-yl]-(2,3,4-trifluorobenzyl)-amine,
4-ethoxy-6-(4-fluorophenyl)-pteridin-2-yl]-(2-trifluoromethylbenzyl)-amine,
4-ethoxy-6-(4-fluorophenyl)-pteridin-2-yl]-(3-trifluoromethylbenzyl)-amine,
4-ethoxy-6-(4-fluorophenyl)-pteridin-2-yl]-(4-trifluoromethylbenzyl)-amine,
4-ethoxy-6-(4-fluorophenyl)-pteridin-2-yl]-(3-trifluoromethoxybenzyl)-amine,
4-ethoxy-6-(4-fluorophenyl)-pteridin-2-yl]-(5-trifluoromethoxybenzyl)-amine,
4-ethoxy-6-(4-fluorophenyl)-pteridin-2-yl]-(2-nitrobenzyl)-amine,
4-ethoxy-6-(4-fluorophenyl)-pteridin-2-yl]-(3-nitrobenzyl)-amine,
4-ethoxy-6-(4-fluorophenyl)-pteridin-2-yl]-(4-nitrobenzyl)-amine,
4-ethoxy-6-(4-fluorophenyl)-pteridin-2-yl]-(3-cyanobenzyl)-amine,
4-ethoxy-6-(4-fluorophenyl)-pteridin-2-yl]-(4-cyanobenzyl)-amine,
4-ethoxy-6-(4-fluorophenyl)-pteridin-2-yl]-(4-dimethylaminobenzyl)-amine,
4-ethoxy-6-(4-fluorophenyl)-pteridin-2-yl]-(4-diethylaminobenzyl)-amine,
4-ethoxy-6-(4-fluorophenyl)-pteridin-2-yl]-(3,4,5-trimethoxybenzyl)-amine,
4-isopropoxy-6-(4-acetamidophenyl)-pteridin-2-yl]-(4-fluoro-benzyl)-amine,
4-isopropoxy-6-(3-acetamidophenyl)-pteridin-2-yl]-(4-fluoro-benzyl)-amine,
4-isopropoxy-6-(4-acetylphenyl)-pteridin-2-yl]-(4-fluoro-benzyl)-amine,
4-isopropoxy-6-(3-acetylphenyl)-pteridin-2-yl]-(4-fluoro-benzyl)-amine,
4-isopropoxy-6-(2-acetylphenyl)-pteridin-2-yl]-(4-fluoro-benzyl)-amine,
4-isopropoxy-6-(5-acetyl-2-chlorophenyl)-pteridin-2-yl]-(4-fluoro-benzyl)-amine,
4-isopropoxy-6-(4-acetyl-3-fluorophenyl)-pteridin-2-yl]-(4-fluoro-benzyl)-amine,
4-isopropoxy-6-(5-acetyl-2-fluorophenyl)-pteridin-2-yl]-(4-fluoro-benzyl)-amine,
4-isopropoxy-6-(3-aminophenyl)-pteridin-2-yl]-(4-fluoro-benzyl)-amine,
4-isopropoxy-6-(4-aminomethylphenyl)-pteridin-2-yl]-(4-fluoro-benzyl)-amine,
4-isopropoxy-6-(4-benzyloxy-2-fluorophenyl)-pteridin-2-yl]-(4-fluoro-benzyl)-amine,
4-isopropoxy-6-(4-benzyloxy-3-fluorophenyl)-pteridin-2-yl]-(4-fluoro-benzyl)-amine,
4-isopropoxy-6-(4-bromophenyl)-pteridin-2-yl]-(4-fluoro-benzyl)-amine,
4-isopropoxy-6-(3-bromophenyl)-pteridin-2-yl]-(4-fluoro-benzyl)-amine,
4-isopropoxy-6-(4-bromo-2,5-dimethylphenyl)-pteridin-2-yl]-(4-fluoro-benzyl)-amine,
4-isopropoxy-6-(2-bromo-5-fluorophenyl)-pteridin-2-yl]-(4-fluoro-benzyl)-amine,
4-isopropoxy-6-(2-bromo-6-fluorophenyl)-pteridin-2-yl]-(4-fluoro-benzyl)-amine,
4-isopropoxy-6-(2-carboxy-5-fluorophenyl)-pteridin-2-yl]-(4-fluoro-benzyl)-amine,
4-isopropoxy-6-(2-carboxyphenyl)-pteridin-2-yl]-(4-fluoro-benzyl)-amine,
4-isopropoxy-6-(4-carboxyphenyl)-pteridin-2-yl]-(4-fluoro-benzyl)-amine,
4-isopropoxy-6-(4-carboxy-2-chlorophenyl)-pteridin-2-yl]-(4-fluoro-benzyl)-amine,
4-isopropoxy-6-(5-carboxy-2-chlorophenyl)-pteridin-2-yl]-(4-fluoro-benzyl)-amine,
4-isopropoxy-6-(4-carboxy-3-chlorophenyl)-pteridin-2-yl]-(4-fluoro-benzyl)-amine,
4-isopropoxy-6-(3-carboxyphenyl)-pteridin-2-yl]-(4-fluoro-benzyl)-amine,
4-isopropoxy-6-(2-chloro-5-formylphenyl)-pteridin-2-yl]-(4-fluoro-benzyl)-amine,
4-isopropoxy-6-(2-chloro-5-hydroxyphenyl)-pteridin-2-yl]-(4-fluoro-benzyl)-amine,
4-isopropoxy-6-(3-chloro-4-fluorophenyl)-pteridin-2-yl]-(4-fluoro-benzyl)-amine,
4-isopropoxy-6-(2-chloro-4-fluorophenyl)-pteridin-2-yl]-(4-fluoro-benzyl)-amine,
4-isopropoxy-6-(4-chloro-2-fluorophenyl)-pteridin-2-yl]-(4-fluoro-benzyl)-amine,
4-isopropoxy-6-(3-chloro-5-methoxyphenyl)-pteridin-2-yl]-(4-fluoro-benzyl)-amine,
4-isopropoxy-6-(2-chloro-4-methylphenyl)-pteridin-2-yl]-(4-fluoro-benzyl)-amine,
4-isopropoxy-6-(2-chloro-5-methylphenyl)-pteridin-2-yl]-(4-fluoro-benzyl)-amine,
4-isopropoxy-6-(3-chloro-5-trifluoromethylphenyl)-pteridin-2-yl]-(4-fluoro-benzyl)-amine,
4-isopropoxy-6-(2-chloro-5-trifluoromethoxyphenyl)-pteridin-2-yl]-(4-fluoro-benzyl)-amine,
4-isopropoxy-6-(4-chloro-2-trifluoromethylphenyl)-pteridin-2-yl]-(4-fluoro-benzyl)-amine,
4-isopropoxy-6-(4-chlorophenyl)-pteridin-2-yl]-(4-fluoro-benzyl)-amine,
4-isopropoxy-6-(3-chlorophenyl)-pteridin-2-yl]-(4-fluoro-benzyl)-amine,
4-isopropoxy-6-(2-chlorophenyl)-pteridin-2-yl]-(4-fluoro-benzyl)-amine,
4-isopropoxy-6-(4-cyanophenyl)-pteridin-2-yl]-(4-fluoro-benzyl)-amine,
4-isopropoxy-6-(3-cyanophenyl)-pteridin-2-yl]-(4-fluoro-benzyl)-amine,
4-isopropoxy-6-(2-cyanophenyl)-pteridin-2-yl]-(4-fluoro-benzyl)-amine,
4-isopropoxy-6-(2,6-dichlorophenyl)-pteridin-2-yl]-(4-fluoro-benzyl)-amine,
4-isopropoxy-6-(3,4-dichlorophenyl)-pteridin-2-yl]-(4-fluoro-benzyl)-amine,
4-isopropoxy-6-(2,6-dichlorophenyl)-pteridin-2-yl]-(4-fluoro-benzyl)-amine,
4-isopropoxy-6-(2,4-dichlorophenyl)-pteridin-2-yl]-(4-fluoro-benzyl)-amine,
4-isopropoxy-6-(2,3-dichlorophenyl)-pteridin-2-yl]-(4-fluoro-benzyl)-amine,
4-isopropoxy-6-(3,5-dichlorophenyl)-pteridin-2-yl]-(4-fluoro-benzyl)-amine,
4-isopropoxy-6-(3,5-difluorophenyl)-pteridin-2-yl]-(4-fluoro-benzyl)-amine,
4-isopropoxy-6-(3,4-difluorophenyl)-pteridin-2-yl]-(4-fluoro-benzyl)-amine,
4-isopropoxy-6-(2,6-difluorophenyl)-pteridin-2-yl]-(4-fluoro-benzyl)-amine,
4-isopropoxy-6-(2,5-difluorophenyl)-pteridin-2-yl]-(4-fluoro-benzyl)-amine,
4-isopropoxy-6-(2,4-difluorophenyl)-pteridin-2-yl]-(4-fluoro-benzyl)-amine,
4-isopropoxy-6-(2,3-difluorophenyl)-pteridin-2-yl]-(4-fluoro-benzyl)-amine,
4-isopropoxy-6-(4-[N,N-dimethylamino]phenyl)-pteridin-2-yl]-(4-fluoro-benzyl)-amine,
4-isopropoxy-6-(2-[N,N-dimethylaminomethyl]phenyl)-pteridin-2-yl]-(4-fluoro-benzyl)-amine,
4-isopropoxy-6-(3,5-dimethylphenyl)-pteridin-2-yl]-(4-fluorobenzyl)-amine,
4-isopropoxy-6-(3,4-dimethylphenyl)-pteridin-2-yl]-(4-fluorobenzyl)-amine,
4-isopropoxy-6-(2,6-dimethylphenyl)-pteridin-2-yl]-(4-fluorobenzyl)-amine,
4-isopropoxy-6-(2,6-dimethoxyphenyl)-pteridin-2-yl]-(4-fluorobenzyl)-amine,
4-isopropoxy-6-(2,5-dimethoxyphenyl)-pteridin-2-yl]-(4-fluorobenzyl)-amine,
4-isopropoxy-6-(2,4-dimethoxyphenyl)-pteridin-2-yl]-(4-fluorobenzyl)-amine,
4-isopropoxy-6-(4-ethoxyphenyl)-pteridin-2-yl]-(4-fluorobenzyl)-amine,
4-isopropoxy-6-(2-ethoxyphenyl)-pteridin-2-yl]-(4-fluorobenzyl)-amine,
4-isopropoxy-6-(4-ethoxycarbonylphenyl)-pteridin-2-yl]-(4-fluorobenzyl)-amine,
4-isopropoxy-6-(3-ethoxycarbonylphenyl)-pteridin-2-yl]-(4-fluorobenzyl)-amine,
4-isopropoxy-6-(4-ethylphenyl)-pteridin-2-yl]-(4-fluorobenzyl)-amine,
4-isopropoxy-6-(4-fluorophenyl)-pteridin-2-yl]-(4-fluorobenzyl)-amine,
4-isopropoxy-6-(3-fluorophenyl)-pteridin-2-yl]-(4-fluorobenzyl)-amine,
4-isopropoxy-6-(2-fluorophenyl)-pteridin-2-yl]-(4-fluorobenzyl)-amine,
4-isopropoxy-6-(3-fluoro-4-formylphenyl)-pteridin-2-yl]-(4-fluorobenzyl)-amine,
4-isopropoxy-6-(4-fluoro-3-formylphenyl)-pteridin-2-yl]-(4-fluorobenzyl)-amine,
4-isopropoxy-6-(4-fluoro-2-methylphenyl)-pteridin-2-yl]-(4-fluorobenzyl)-amine,
4-isopropoxy-6-(2-fluoro-5-methylphenyl)-pteridin-2-yl]-(4-fluorobenzyl)-amine,
4-isopropoxy-6-(2-fluoro-5-methoxyphenyl)-pteridin-2-yl]-(4-fluorobenzyl)-amine,
4-isopropoxy-6-(5-fluoro-2-methoxycarbonylphenyl)-pteridin-2-yl]-(4-fluorobenzyl)-amine,
4-isopropoxy-6-(2-formyl-5-methoxyphenyl)-pteridin-2-yl]-(4-fluorobenzyl)-amine,
4-isopropoxy-6-(5-formyl-2-methoxyphenyl)-pteridin-2-yl]-(4-fluorobenzyl)-amine,
4-isopropoxy-6-(2-formyl-5-methylphenyl)-pteridin-2-yl]-(4-fluorobenzyl)-amine,
4-isopropoxy-6-(2-formylphenyl)-pteridin-2-yl]-(4-fluorobenzyl)-amine,
4-isopropoxy-6-(3-formylphenyl)-pteridin-2-yl]-(4-fluorobenzyl)-amine,
4-isopropoxy-6-(4-formylphenyl)-pteridin-2-yl]-(4-fluorobenzyl)-amine,
4-isopropoxy-6-(3-hydroxy-4-methoxycarbonylphenyl)-pteridin-2-yl]-(4-fluorobenzyl)-amine,
4-isopropoxy-6-(3-hydroxymethylphenyl)-pteridin-2-yl]-(4-fluorobenzyl)-amine,
4-isopropoxy-6-(4-hydroxymethylphenyl)-pteridin-2-yl]-(4-fluorobenzyl)-amine,
4-isopropoxy-6-(4-hydroxyphenyl)-pteridin-2-yl]-(4-fluorobenzyl)-amine,
4-isopropoxy-6-(3-hydroxyphenyl)-pteridin-2-yl]-(4-fluorobenzyl)-amine,
4-isopropoxy-6-(4-iodophenyl)-pteridin-2-yl]-(4-fluorobenzyl)-amine,
4-isopropoxy-6-(3-iodophenyl)-pteridin-2-yl]-(4-fluorobenzyl)-amine,
4-isopropoxy-6-(4-isopropoxycarbonylphenyl)-pteridin-2-yl]-(4-fluorobenzyl)-amine,
4-isopropoxy-6-(3-isopropoxycarbonylphenyl)-pteridin-2-yl]-(4-fluorobenzyl)-amine,
4-isopropoxy-6-(4-methanesulfonylphenyl)-pteridin-2-yl]-(4-fluorobenzyl)-amine,
4-isopropoxy-6-(4-methoxy-2-formylphenyl)-pteridin-2-yl]-(4-fluorobenzyl)-amine,
4-isopropoxy-6-(4-methoxycarbonylphenyl)-pteridin-2-yl]-(4-fluorobenzyl)-amine,
4-isopropoxy-6-(3-methoxycarbonylphenyl)-pteridin-2-yl]-(4-fluorobenzyl)-amine,
4-isopropoxy-6-(4-methoxyphenyl)-pteridin-2-yl]-(4-fluorobenzyl)-amine,
4-isopropoxy-6-(3-methoxyphenyl)-pteridin-2-yl]-(4-fluorobenzyl)-amine,
4-isopropoxy-6-(2-methoxyphenyl)-pteridin-2-yl]-(4-fluorobenzyl)-amine,
4-isopropoxy-6-(3,4-methylenedioxyphenyl)-pteridin-2-yl]-(4-fluorobenzyl)-amine,
4-isopropoxy-6-(4-methylphenyl)-pteridin-2-yl]-(4-fluorobenzyl)-amine,
4-isopropoxy-6-(2-methylphenyl)-pteridin-2-yl]-(4-fluorobenzyl)-amine,
4-isopropoxy-6-(4-(methylthio)phenyl)-pteridin-2-yl]-(4-fluorobenzyl)-amine,
4-isopropoxy-6-(3-(methylthio)phenyl)-pteridin-2-yl]-(4-fluorobenzyl)-amine,
4-isopropoxy-6-(4-morpholinophenyl)-pteridin-2-yl]-(4-fluorobenzyl)-amine,
4-isopropoxy-6-(3-nitrophenyl)-pteridin-2-yl]-(4-fluorobenzyl)-amine,
4-isopropoxy-6-(4-phenoxyphenyl)-pteridin-2-yl]-(4-fluorobenzyl)-amine,
4-isopropoxy-6-(4-(tert-butoxycarbonyl)phenyl)-pteridin-2-yl]-(4-fluoro-benzyl)-amine,
4-isopropoxy-6-(3-(tert-butoxycarbonyl)phenyl)-pteridin-2-yl]-(4-fluoro-benzyl)-amine,
4-isopropoxy-6-(2-(tert-butoxycarbonyl)phenyl)-pteridin-2-yl]-(4-fluoro-benzyl)-amine,
4-isopropoxy-6-(4-(tert-butoxycarbonylamino)-3-methoxyphenyl)-pteridin-2-yl]-(4-fluorobenzyl)-amine,
4-isopropoxy-6-(4-(tert-butylphenyl)-pteridin-2-yl]-(4-fluorobenzyl)-amine,
4-isopropoxy-6-(4-(2-thienyl)phenyl)-pteridin-2-yl]-(4-fluorobenzyl)-amine,
4-isopropoxy-6-(4-trifluoromethoxyphenyl)-pteridin-2-yl]-(4-fluorobenzyl)-amine,
4-isopropoxy-6-(2,4,6-trimethylphenyl)-pteridin-2-yl]-(4-fluorobenzyl)-amine,
4-isopropoxy-6-(3,4,5-trifluorophenyl)-pteridin-2-yl]-(4-fluorobenzyl)-amine,
4-isopropoxy-6-(3-trifluoromethoxyphenyl)-pteridin-2-yl]-(4-fluorobenzyl)-amine,
4-isopropoxy-6-(3-trifluoromethylphenyl)-pteridin-2-yl]-(4-fluorobenzyl)-amine,
4-isopropoxy-6-(2-trifluoromethylphenyl)-pteridin-2-yl]-(4-fluorobenzyl)-amine,
4-isopropoxy-6-(3,4,5-trimethoxyphenyl)-pteridin-2-yl]-(4-fluorobenzyl)-amine,
4-isopropoxy-6-(4-vinylphenyl)-pteridin-2-yl]-(4-fluorobenzyl)-amine,
4-isopropoxy-6-(2-acetamidopyridin-5-yl)-pteridin-2-yl]-(4-fluorobenzyl)-amine,
4-isopropoxy-6-(2-benzothienyl)-pteridin-2-yl]-(4-fluorobenzyl)-amine,
4-isopropoxy-6-(1-benzothiophen-3-yl)-pteridin-2-yl]-(4-fluorobenzyl)-amine,
4-isopropoxy-6-(1-benzothiophen-2-yl)-pteridin-2-yl]-(4-fluorobenzyl)-amine,
4-isopropoxy-6-(2-bromo-3-chloropyridin-4-yl)-pteridin-2-yl]-(4-fluoro-benzyl)-amine,
4-isopropoxy-6-(2-bromo-3-methylpyridin-5-yl)-pteridin-2-yl]-(4-fluoro-benzyl)-amine,
4-isopropoxy-6-(2-bromopyridin-5-yl)-pteridin-2-yl]-(4-fluorobenzyl)-amine,
4-isopropoxy-6-(2-chloro-3-methylpyridin-5-yl)-pteridin-2-yl]-(4-fluoro-benzyl)-amine,
4-isopropoxy-6-(2-chloro-6-isopropylpyridin-3-yl)-pteridin-2-yl]-(4-fluoro-benzyl)-amine,
4-isopropoxy-6-(5-chlorothien-2-yl)-pteridin-2-yl]-(4-fluoro-benzyl)-amine,
4-isopropoxy-6-(5-bromothien-2-yl)-pteridin-2-yl]-(4-fluoro-benzyl)-amine,
4-isopropoxy-6-(2-chloro-3-fluoropyridin-4-yl)-pteridin-2-yl]-(4-fluoro-benzyl)-amine,
4-isopropoxy-6-(2,5-dibromopyridin-3-yl)-pteridin-2-yl]-(4-fluoro-benzyl)-amine,
4-isopropoxy-6-(2,6-dichloropyridin-3-yl)-pteridin-2-yl]-(4-fluoro-benzyl)-amine,
4-isopropoxy-6-(2,4-dimethoxypyrimidin-5-yl)-pteridin-2-yl]-(4-fluoro-benzyl)-amine,
4-isopropoxy-6-(3,5-dimethylisoxazol-4-yl)-pteridin-2-yl]-(4-fluoro-benzyl)-amine,
4-isopropoxy-6-(2,4-dioxo-1,2,3,4-tetrahydropyrimidin-5-yl)-pteridin-2-yl]-(4-fluoro-benzyl)-amine,
4-isopropoxy-6-(2-ethoxypyridin-3-yl)-pteridin-2-yl]-(4-fluorobenzyl)-amine,
4-isopropoxy-6-(2-fluoro-3-methylpyridin-5-yl)-pteridin-2-yl]-(4-fluorobenzyl)-amine,
4-isopropoxy-6-(2-fluoropyridin-3-yl)-pteridin-2-yl]-(4-fluorobenzyl)-amine,
4-isopropoxy-6-(2-fluoropyridin-5-yl)-pteridin-2-yl]-(4-fluorobenzyl)-amine,
4-isopropoxy-6-(5-formyl-2-furyl)-pteridin-2-yl]-(4-fluorobenzyl)-amine,
4-isopropoxy-6-(5-formyl-thiophen-2-yl)-pteridin-2-yl]-(4-fluorobenzyl)-amine,
4-isopropoxy-6-(furan-3-yl)-pteridin-2-yl]-(4-fluorobenzyl)-amine,
4-isopropoxy-6-(furan-2-yl)-pteridin-2-yl]-(4-fluorobenzyl)-amine,
4-isopropoxy-6-(indol-5-yl)-pteridin-2-yl]-(4-fluorobenzyl)-amine,
4-isopropoxy-6-(isoquinolin-4-yl)-pteridin-2-yl]-(4-fluorobenzyl)-amine,
4-isopropoxy-6-(5-methylfuran-2-yl)-pteridin-2-yl]-(4-fluorobenzyl)-amine,
4-isopropoxy-6-(methoxypyrimidin-5-yl)-pteridin-2-yl]-(4-fluorobenzyl)-amine,
4-isopropoxy-6-(5-methyl-1-benzothiophen-2-yl)-pteridin-2-yl]-(4-fluorobenzyl)-amine,
4-isopropoxy-6-(5-methyl-3-phenyl-4-isoxazolyl)-pteridin-2-yl]-(4-fluorobenzyl)-amine,
4-isopropoxy-6-(3-methylpyridin-2-yl)-pteridin-2-yl]-(4-fluorobenzyl)-amine,
4-isopropoxy-6-(5-methylpyridin-2-yl)-pteridin-2-yl]-(4-fluorobenzyl)-amine,
4-isopropoxy-6-(5-methylpyridin-3-yl)-pteridin-2-yl]-(4-fluorobenzyl)-amine,
4-isopropoxy-6-(2-methoxypyridin-3-yl)-pteridin-2-yl]-(4-fluorobenzyl)-amine,
4-isopropoxy-6-(2-methoxypyridin-5-yl)-pteridin-2-yl]-(4-fluorobenzyl)-amine,
4-isopropoxy-6-(pyridin-4-yl)-pteridin-2-yl]-(4-fluorobenzyl)-amine,
4-isopropoxy-6-(pyridin-3-yl)-pteridin-2-yl]-(4-fluorobenzyl)-amine,
4-isopropoxy-6-(pyrimidin-5-yl)-pteridin-2-yl]-(4-fluorobenzyl)-amine,
4-isopropoxy-6-(quinolin-3-yl)-pteridin-2-yl]-(4-fluorobenzyl)-amine,
4-isopropoxy-6-(quinolin-8-yl)-pteridin-2-yl]-(4-fluorobenzyl)-amine,
4-isopropoxy-6-(thien-2-yl)-pteridin-2-yl]-(4-fluorobenzyl)-amine,
4-isopropoxy-6-(4-fluorophenyl)-pteridin-2-yl]-benzylamine,
4-isopropoxy-6-(4-fluorophenyl)-pteridin-2-yl]-(4-methylbenzyl)-amine,
4-isopropoxy-6-(4-fluorophenyl)-pteridin-2-yl]-(3-methylbenzyl)-amine,
4-isopropoxy-6-(4-fluorophenyl)-pteridin-2-yl]-(2-methylbenzyl)-amine,
4-isopropoxy-6-(4-fluorophenyl)-pteridin-2-yl]-(4-methoxybenzyl)-amine,
4-isopropoxy-6-(4-fluorophenyl)-pteridin-2-yl]-(3-methoxybenzyl)-amine,
4-isopropoxy-6-(4-fluorophenyl)-pteridin-2-yl]-(2-methoxybenzyl)-amine,
4-isopropoxy-6-(4-fluorophenyl)-pteridin-2-yl]-(2,5-dihydroxybenzyl)-amine,
4-isopropoxy-6-(4-fluorophenyl)-pteridin-2-yl]-(4-propoxybenzyl)-amine,
4-isopropoxy-6-(4-fluorophenyl)-pteridin-2-yl]-(4-phenoxybenzyl)-amine,
4-isopropoxy-6-(4-fluorophenyl)-pteridin-2-yl]-(3-[3,4-dichlorophenoxy]-benzyl)-amine,
4-isopropoxy-6-(4-fluorophenyl)-pteridin-2-yl]-(3-[3,5-dichlorophenoxy]-benzyl)-amine,
4-isopropoxy-6-(4-fluorophenyl)-pteridin-2-yl]-(2-bromobenzyl)-amine,
4-isopropoxy-6-(4-fluorophenyl)-pteridin-2-yl]-(3-bromobenzyl)-amine,
4-isopropoxy-6-(4-fluorophenyl)-pteridin-2-yl]-(4-bromobenzyl)-amine,
4-isopropoxy-6-(4-fluorophenyl)-pteridin-2-yl]-(2-chlorobenzyl)-amine,
4-isopropoxy-6-(4-fluorophenyl)-pteridin-2-yl]-(3-chlorobenzyl)-amine,
4-isopropoxy-6-(4-fluorophenyl)-pteridin-2-yl]-(4-chlorobenzyl)-amine,
4-isopropoxy-6-(4-fluorophenyl)-pteridin-2-yl]-(2-fluorobenzyl)-amine,
4-isopropoxy-6-(4-fluorophenyl)-pteridin-2-yl]-(3-fluorobenzyl)-amine,
4-isopropoxy-6-(4-fluorophenyl)-pteridin-2-yl]-(2,3-dichlorobenzyl)-amine,
4-isopropoxy-6-(4-fluorophenyl)-pteridin-2-yl]-(2,4-dichlorobenzyl)-amine,
4-isopropoxy-6-(4-fluorophenyl)-pteridin-2-yl]-(2,6-dichlorobenzyl)-amine,
4-isopropoxy-6-(4-fluorophenyl)-pteridin-2-yl]-(3,4-dichlorobenzyl)-amine,
4-isopropoxy-6-(4-fluorophenyl)-pteridin-2-yl]-(3,5-dichlorobenzyl)-amine,
4-isopropoxy-6-(4-fluorophenyl)-pteridin-2-yl]-(2,3-difluorobenzyl)-amine,
4-isopropoxy-6-(4-fluorophenyl)-pteridin-2-yl]-(2,4-difluorobenzyl)-amine,
4-isopropoxy-6-(4-fluorophenyl)-pteridin-2-yl]-(2,5-difluorobenzyl)-amine,
4-isopropoxy-6-(4-fluorophenyl)-pteridin-2-yl]-(2,6-difluorobenzyl)-amine,
4-isopropoxy-6-(4-fluorophenyl)-pteridin-2-yl]-(3,4-difluorobenzyl)-amine,
4-isopropoxy-6-(4-fluorophenyl)-pteridin-2-yl]-(3,5-difluorobenzyl)-amine,
4-isopropoxy-6-(4-fluorophenyl)-pteridin-2-yl]-(2,3,4-trifluorobenzyl)-amine,
4-isopropoxy-6-(4-fluorophenyl)-pteridin-2-yl]-(2-trifluoromethylbenzyl)-amine,
4-isopropoxy-6-(4-fluorophenyl)-pteridin-2-yl]-(3-trifluoromethylbenzyl)-amine,
4-isopropoxy-6-(4-fluorophenyl)-pteridin-2-yl]-(4-trifluoromethylbenzyl)-amine,
4-isopropoxy-6-(4-fluorophenyl)-pteridin-2-yl]-(3-trifluoromethoxybenzyl)-amine,
4-isopropoxy-6-(4-fluorophenyl)-pteridin-2-yl]-(5-trifluoromethoxybenzyl)-amine,
4-isopropoxy-6-(4-fluorophenyl)-pteridin-2-yl]-(2-nitrobenzyl)-amine,
4-isopropoxy-6-(4-fluorophenyl)-pteridin-2-yl]-(3-nitrobenzyl)-amine,
4-isopropoxy-6-(4-fluorophenyl)-pteridin-2-yl]-(4-nitrobenzyl)-amine,
4-isopropoxy-6-(4-fluorophenyl)-pteridin-2-yl]-(3-cyanobenzyl)-amine,
4-isopropoxy-6-(4-fluorophenyl)-pteridin-2-yl]-(4-cyanobenzyl)-amine,
4-isopropoxy-6-(4-fluorophenyl)-pteridin-2-yl]-(4-dimethylaminobenzyl)-amine,
4-isopropoxy-6-(4-fluorophenyl)-pteridin-2-yl]-(4-diethylaminobenzyl)-amine,
4-isopropoxy-6-(4-fluorophenyl)-pteridin-2-yl]-(3,4,5-trimethoxybenzyl)-amine,
2-{[6-(4-fluorophenyl)-4-(2,2,2-trifluoroethylamino)-pteridin-2-ylamino]-methyl}-chlorobenzene,
4-{[6-(4-fluorophenyl)-4-(2,2,2-trifluoroethylamino)-pteridin-2-ylamino]-methyl}-chlorobenzene,
4-{[6-(4-fluorophenyl)-4-(2,2,2-trifluoroethylamino)-pteridin-2-ylamino]-methyl}-methylbenzene,
4-{[6-(4-fluorophenyl)-4-(2,2,2-trifluoroethylamino)-pteridin-2-ylamino]-methyl}-methoxybenzene,
3-{[6-(4-fluorophenyl)-4-(2,2,2-trifluoroethylamino)-pteridin-2-ylamino]-methyl}-methylbenzene,
2-{[6-(4-fluorophenyl)-4-(2,2,2-trifluoroethylamino)-pteridin-2-ylamino]-methyl}-methylbenzene,
2-{[6-(4-fluorophenyl)-4-(2,2,2-trifluoroethylamino)-pteridin-2-ylamino]-methyl}-methoxybenzene,
3-{[6-(4-fluorophenyl)-4-(2,2,2-trifluoroethylamino)-pteridin-2-ylamino]-methyl}-methoxybenzene,
3-{[6-(4-fluorophenyl)-4-(2,2,2-trifluoroethylamino)-pteridin-2-ylamino]-methyl}-fluorobenzene,
2-{[6-(4-fluorophenyl)-4-(2,2,2-trifluoroethylamino)-pteridin-2-ylamino]-methyl}-fluorobenzene,
4-{[6-(4-fluorophenyl)-4-(2,2,2-trifluoroethylamino)-pteridin-2-ylamino]-methyl}-fluorobenzene,
3-{[6-(4-fluorophenyl)-4-(2,2,2-trifluoroethylamino)-pteridin-2-ylamino]-methyl}-chlorobenzene,
4-{[6-(4-fluorophenyl)-4-(2,2,2-trifluoroethylamino)-pteridin-2-ylamino]-methyl}-trifluoromethoxybenzene,
2-{[6-(4-fluorophenyl)-4-(2,2,2-trifluoroethylamino)-pteridin-2-ylamino]-methyl}-trifluoromethoxybenzene,
4-{[6-(4-fluorophenyl)-4-(2,2,2-trifluoroethylamino)-pteridin-2-ylamino]-methyl}-trifluoromethylbenzene,
2-{[6-(4-fluorophenyl)-4-(2,2,2-trifluoroethylamino)-pteridin-2-ylamino]-methyl}-trifluoromethylbenzene,
3-{[6-(4-fluorophenyl)-4-(2,2,2-trifluoroethylamino)-pteridin-2-ylamino]-methyl}-trifluoromethylbenzene,
2-{[6-(4-fluorophenyl)-4-(2,2,2-trifluoroethylamino)-pteridin-2-ylamino]-methyl}-bromobenzene,
4-{[6-(4-fluorophenyl)-4-(2,2,2-trifluoroethylamino)-pteridin-2-ylamino]-methyl}-bromobenzene,
3-{[6-(4-fluorophenyl)-4-(2,2,2-trifluoroethylamino)-pteridin-2-ylamino]-methyl}-bromobenzene,
3-{[6-(4-fluorophenyl)-4-(2,2,2-trifluoroethylamino)-pteridin-2-ylamino]-methyl}-iodobenzene,
2-{[6-(4-fluorophenyl)-4-(2,2,2-trifluoroethylamino)-pteridin-2-ylamino]-methyl}-hydroxybenzene,
4-{[6-(4-fluorophenyl)-4-(2,2,2-trifluoroethylamino)-pteridin-2-ylamino]-4-methyl}-thien-2-ylbenzene,
4-{[6-(4-fluorophenyl)-4-(2,2,2-trifluoroethylamino)-pteridin-2-ylamino]-methyl}-morpholinobenzene,
2-{[6-(4-fluorophenyl)-4-(2,2,2-trifluoroethylamino)-pteridin-2-ylamino]-methyl}-morpholinobenzene,
4-{[6-(4-fluorophenyl)-4-(2,2,2-trifluoroethylamino)-pteridin-2-ylamino]-methyl}-morpholinomethylbenzene,
4-{[6-(4-fluorophenyl)-4-(2,2,2-trifluoroethylamino)-pteridin-2-ylamino]-methyl}-(4-methylpiperazino)benzene,
2-{[6-(4-fluorophenyl)-4-(2,2,2-trifluoroethylamino)-pteridin-2-ylamino]-methyl}-methylthiobenzene,
3-{[6-(4-fluorophenyl)-4-(2,2,2-trifluoroethylamino)-pteridin-2-ylamino]-methyl}-phenoxymethylbenzene,
4-{[6-(4-fluorophenyl)-4-(2,2,2-trifluoroethylamino)-pteridin-2-ylamino]-methyl}-(pyrrolidin-1-ylmethyl)benzene,
2-{[6-(4-fluorophenyl)-4-(2,2,2-trifluoroethylamino)-pteridin-2-ylamino]-methyl}-(pyrrolidin-1-ylmethyl)benzene, and
3-{[6-(4-fluorophenyl)-4-(2,2,2-trifluoroethylamino)-pteridin-2-ylamino]-methyl}-(pyrimidin-2-yl)benzene.

61. A pteridine derivative being 2-amino-4-isopropoxy-6-(4-fluorophenyl)-pteridine.

62. A method of treatment or prevention of an infection due to a virus from the Flaviridae family, consisting essentially of administering to a patient in need thereof a therapeutically effective amount of a pteridine derivative selected from the group consisting of 2-amino-4-ethoxy-6-(4-fluorophenyl)-pteridine and 2-amino-4-isopropoxy-6-(4-fluorophenyl)-pteridine.
